(19) United States
(12) Patent Application Publication
(10) Pub. No.: US 2022/0267431 A1
(43) Pub. Date: Aug. 25, 2022

(54) Patient Populations Amenable to IL23-Antagonist Therapy

(71) Applicant: ASTRAZENECA COLLABORATION VENTURES, LLC, Wilmington (DE)

(72) Inventor: Qing Zhou, Madison, NJ (US)

(21) Appl. No.: 17/441,010

(22) PCT Filed: Mar. 26, 2020
(86) PCT No.: PCT/US20/24990

§ 371 Date: Sep. 20, 2021

Related U.S. Application Data
(60) Provisional application No. 62/824,245, filed on Mar. 26, 2019.

(51) Int. Cl. C07K 16/24; A61P 1/12; A61P 37/06; G01N 33/68

(52) U.S. Cl. CPC C07K 16/244; A61P 1/12; G01N 2800/065; G01N 33/6893; G01N 2470/06; A61P 37/06

(57) ABSTRACT

The disclosure provides methods for selecting patient sub-populations, or subjects, with inflammatory conditions such as inflammatory bowel diseases that are amenable to treatment with anti-Interleukin-23 therapy by measuring the serum levels of Interleukin-22 Binding Protein and/or the serum levels of Interferon-γ. In addition, the methods are useful in identifying sub-populations of patients with inflammatory disorders, such as psoriasis, psoriatic arthritis, rheumatoid arthritis, ankylosing spondylitis that are amenable to treatment with an anti-IL-23 therapy and/or an anti-IFN-γ therapy.

### CROSS-REFERENCE TO RELATED APPLICATIONS

This application claims priority to Provisional U.S. Patent Application No. 62/824,245, filed Mar. 26, 2019, which is hereby incorporated by reference in its entirety.

### FIELD

The disclosure relates generally to the field of selecting patient sub-populations amenable to treatment of inflammatory conditions and, more particularly, amenable to treatment of inflammatory bowel diseases.

### BACKGROUND

Interleukin-22 (IL-22) is an IL-10 cytokine family member strongly expressed in both Crohn's disease (CD) and ulcerative colitis (UC). Consistent with these observations, IL-22 has been shown to have pro-inflammatory properties, but this cytokine has also been shown to exert organ-protective effects in liver and lung. Sonnenberg et al., J. Exp. Med. 207:1293-1305 (2010); Cobleigh et al., Am. J. Pathol. 182:21-28 (2013). IL-22 is induced by various environmental and endogenous signals, such as IL-23. The identification of IL23R as a susceptibility gene by several genome-wide association studies is consistent with the IL-23 pathway having a role in Inflammatory bowel diseases (IBDs). IL-22 is up-regulated in the intestine in patients with IBD. IL-22 is normally able to promote mucosal healing in the intestine; however, when uncontrolled, it can lead to intestinal pathogenesis. Therefore, tight control of IL-22 activity is essential. IL-22 binding protein (IL-22BP) exerts this control by specifically binding IL-22 and preventing it from binding membrane-bound IL-22 receptor 1 (IL-22R1). The binding affinity of IL-22 and IL-22BP is 20- to 1000-fold that of the former and membrane-bound IL-22R1. IL-22 and IL-22BP exhibit an inverse expression pattern upon tissue damage in the intestine in mouse models: IL-22BP is most highly expressed in the colon during homeostasis and tissue repair, whereas IL-22 is most highly expressed at the peak of tissue damage. Thus, careful regulation of IL-22 and IL-22BP controls homeostasis in the intestine, but the role of IL-22BP in human IBDs is uncertain.

Interferon-gamma (IFN-7) has been identified as a classic Th1 cytokine promoting inflammatory responses, but its role in various inflammatory conditions is not yet clear. Moreover, IFN-γ is known to play a role in both innate and adaptive immune responses; it also plays a role in defending against microbial infection, including viral and some bacterial and protozoan infections. IFN-γ is stimulated by IL-23, a key mediator of inflammatory responses, and IFN-γ has been identified as a causative factor in inflammatory bowel disease (Ito et al., Clin. and Exper. Immunol. 146:330-338 (2006)). Consistent with these observations, Strober et al., reported that IFN-γ and IL-17/IL-22 were important cytokines involved in inflammatory bowel diseases (IBDs). In addition, Fontolizumab, an anti-IFN-γ antibody, was shown to decrease the severity of Crohn's disease. Ghosh et al., Gut 55:1071-1073 (2006). Harbour et al., Proc. Natl. Acad. Sci. USA 112:7061-7066 (2015) appeared to confirm this pro-inflammatory view of IFN-γ in analyzing Th17 T-cells and showing that a subset of the Th17 cells differentiated into Th1 cells, the cells for which IFN-γ is a defining marker. Harbour et al. also disclosed, however, that induction of colitis required expression of Sta4 and T-bet, but not IFN-γ. This latter finding of Harbour et al. was not surprising in view of a report that IFN-γ has anti-inflammatory properties early in colitis. Sheikh et al., J. Immunol. 184:4069-4073 (2010). Thus, accumulating information on the role of IFN-γ in inflammatory responses establishes the cytokine as having a significant role, but the precise nature of that role has not yet been fully elucidated.

Interleukin (IL)-23 is a pro-inflammatory cytokine implicated in the pathogenesis of various inflammatory conditions including, but not limited to, Crohn's disease (CD), ulcerative colitis (UC), psoriasis, psoriatic arthritis, rheumatoid arthritis, and ankylosing spondylitis. IL23 inducesT-cells to express a number of inflammatory genes, including IL-17A, IL-17A receptor, TNF-α, and GM-CSF. The main known effects of IL-23 are to drive the differentiation of T helper Th17 cells, as well as macrophages, natural killer (NK) cells, dendritic cells, and innate lymphoid cells leading to up-regulation of IL-17, IL-22, TNF-α, GM-CSF, and IFN-γ, as well as down-regulation of IL-10.

IL-23, a member of the IL-12 family of cytokines, is a heterodimeric cytokine that potently induces pro-inflammatory cytokines. IL-23 is related to the heterodimeric cytokine Interleukin 12 (IL-12) both sharing a common p40 subunit. In IL-23, a unique p19 subunit is covalently bound to the p40 subunit. In IL-12, the unique subunit is p35 (Oppmann et al., Immunity, 2000, 13: 713-715). IL-23 is expressed by antigen presenting cells (such as dendritic cells and macrophages) in response to activation stimuli such as CD40 ligation, Toll-like receptor agonists and pathogens. IL-23 binds a heterodimeric receptor comprising an IL-12Rβ1 subunit (which is shared with the IL-12 receptor) and a unique receptor subunit, IL-23R.

IL-23 acts on activated and memory T cells and promotes survival and expansion of the T cell subset, Th17. Th17 cells produce pro-inflammatory cytokines, including IL-6, IL-17, TNFα, IL-22 and GM-CSF. IL-23 also acts on natural killer cells, dendritic cells and macrophages to induce pro-inflammatory cytokine expression. Unlike IL-23, IL-12 induces the differentiation of naïve CD4+ T cells into mature Th1 IFN-γ-producing effector cells, and induces NK and cytotoxic T cell function by stimulating IFN-γ production. Th1 cells driven by IL-12 were previously thought to be the pathogenic T cell subset in many autoimmune diseases; however, more recent animal studies in models of inflammatory bowel disease, psoriasis, inflammatory arthritis and multiple sclerosis, in which the individual contributions of IL-12 and IL-23 were evaluated, have firmly established that IL-23, not IL-12, is the key driver in autoimmune/inflammatory disease (Ahem et al., Immun. Rev. 2008 226:147-159; Cua et al., Nature 2003 421:744-748; Yago et al., Arthritis Res and Ther. 2007 9(5): R96). It is believed that IL-12 plays a critical role in the development of protective innate and adaptive immune responses to many intracellular pathogens and viruses and in tumor immune surveillance. See Kastelein, et al., Annual Review of Immunology, 2007, 25: 221-42; Liu, et al., Rheumatology, 2007, 46(8): 1266-73; Bowman et al., Current Opinion in Infectious Diseases, 2006 19:245-52; Fieschi and Casanova, Eur. J. Immunol. 2003 33:1461-4; Meeran et al., Mol. Cancer Ther. 2006 5: 825-32; Langowski et al., Nature 2006 442: 461-5. As such, IL-23 specific inhibition (sparing IL-12 or the shared p40 subunit) is expected to have a superior safety profile compared to dual inhibition of IL-12 and IL-23.

IL-23 is related to the heterodimeric cytokine Interleukin 12 (IL-12) both sharing a common p40 subunit. In IL-23, a unique p19 subunit is covalently bound to the p40 subunit. In IL-12, the unique subunit is p35 (Oppmann et al., Immunity, 2000, 13: 713-715). The IL-23 heterodimeric protein is secreted. Like IL-12, IL-23 is expressed by antigen presenting cells (such as dendritic cells and macrophages) in response to activation stimuli such as CD40 ligation, Toll-like receptor agonists and pathogens. IL-23 binds a heterodimeric receptor comprising an IL-12R131 subunit (which is shared with the IL-12 receptor) and a unique receptor subunit, IL-23R. The IL-12 receptor consists of IL-12W and IL-12R132. IL-23 binds its heterodimeric receptor and signals through JAK2 and Tyk2 to activate STAT1, 3, 4 and 5 (Parham et al., J. Immunol. 2002, 168:5699-708). The subunits of the receptor are predominantly co-expressed on activated or memory T cells and natural killer cells and also at lower levels on dendritic cells, monocytes, macrophages, microglia, keratinocytes and synovial fibroblasts. IL-23 and IL-12 act on different T cell subsets and play substantially different roles in vivo.

IL-23 acts on activated and memory T cells and promotes survival and expansion of the T cell subset, Th17. Th17 cells produce proinflammatory cytokines including IL-6, IL-17, TNF-α, IL-22 and GM-CSF. IL-23 also acts on natural killer cells, dendritic cells and macrophages to induce pro-inflammatory cytokine expression. Unlike IL-23, IL-12 induces the differentiation of naïve CD4+ T cells into mature Th1 IFN-γ-producing effector cells, and induces NK and cytotoxic T cell function by stimulating IFN-γ production. Th1 cells driven by IL-12 were previously thought to be the pathogenic T cell subset in many autoimmune diseases, however, more recent animal studies in models of inflammatory bowel disease, psoriasis, inflammatory arthritis and multiple sclerosis, in which the individual contributions of IL-12 versus IL-23 were evaluated have firmly established that IL-23, not IL-12, is the key driver in autoimmune/inflammatory disease (Ahem et al., Immun. Rev. 2008 226:147-159; Cua et al., Nature 2003 421:744-748; Yago et al., Arthritis Res and Ther. 2007 9(5): R96). It is believed that IL-12 plays a critical role in the development of protective innate and adaptive immune responses to many intracellular pathogens and viruses and in tumor immune surveillance. See Kastelein, et al., Annual Review of Immunology, 2007, 25: 221-42; Liu, et al., Rheumatology, 2007, 46(8): 1266-73; Bowman et al., Current Opinion in Infectious Diseases, 2006 19:245-52; Fieschi and Casanova, Eur. J. Immunol. 2003 33:1461-4; Meeran et al., Mol. Cancer Ther. 2006 5: 825-32; and Langowski et al., Nature 2006 442: 461-5. As such, IL-23 specific inhibition (sparing IL-12 or the shared p40 subunit) should have a potentially superior safety profile compared to dual inhibition of IL-12 and IL-23.

Use of IL-23 specific antagonists that inhibit human IL-23 (such as antibodies that bind at least the unique p19 subunit or bind both the p19 and p40 subunits of IL-23) that spare IL-12 should provide efficacy equal to or greater than IL-12 antagonists or p40 antagonists without the potential risks associated with inhibition of IL-12. Murine, humanized and phage display antibodies selected for inhibition of recombinant IL-23 have been described; see, for example, U.S. Pat. No. 7,491,391, WIPO Publications WO1999/05280, WO2007/0244846, WO2007/027714, WO 2007/076524, WO2007/147019, WO2008/103473, WO 2008/103432, WO2009/043933 and WO2009/082624. Fully human therapeutic agents that are able to inhibit native human IL-23 would be highly specific for the target, particularly in vivo. Complete inhibition of the in vivo target can result in lower dose formulations, less frequent and/or more effective dosing which in turn results in reduced cost and increased efficiency.

In view of the persistent incidence of inflammatory conditions, such as inflammatory bowel diseases, in the human population, and in view of the incomplete understanding of inflammation as a biological process, it is apparent that a need continues to exist for efficiently identifying subjects or patients that are amenable to particular treatments for such disorders and diseases, particularly where the treatments are efficacious and cost-effective.

### SUMMARY

The disclosure provides effective methods for identifying patient populations, or sub-populations, amenable to anti-cytokine therapy in the form of anti-IL-23 agents to treat inflammatory conditions. Disclosed herein are data establishing that subjects with inflammatory conditions that exhibit elevated levels of IFN-γ are more likely responsive to treatment with an anti-Interleukin-23 agent, such as an anti-IL-23 antibody, e.g., Brazikumab. Accordingly, the disclosure provides methods for selecting subjects with inflammatory conditions, such as inflammatory bowel diseases, that are responsive to treatment with anti-Interleukin-23 therapy by measuring the serum levels of Interleukin-22 Binding Protein and/or the serum levels of IFN-γ. In addition, the methods are useful in identifying sub-populations of patients with inflammatory disorders, such as psoriasis, psoriatic arthritis, rheumatoid arthritis, and ankylosing spondylitis, that are amenable to treatment with an anti-IL-23 therapy and/or an anti-IFN-γ therapy.

One aspect of the disclosure is based on the pro-inflammatory properties of IL-22 and not on its organ-protective functions in providing a method of selecting a subject with an inflammatory condition amenable to treatment with an anti-Interleukin-23 (anti-IL-23) agent, comprising: (a) measuring the serum level of Interleukin-22 Binding Protein (IL-22BP) in a subject; (b) comparing the serum level of IL-22BP in the subject to a serum level of IL-22BP in a control, wherein the control is one or more individuals without an inflammatory condition; and (c) selecting the subject as having an inflammatory condition amenable to treatment with an anti-IL-23 agent if the serum level of IL-22BP is lower in the subject than in the control. Apparent from a consideration of this aspect of the disclosure is that the methods are based, in part, on the pro-inflammatory properties of IL-22 and the antagonism thereof provided by IL-22BP, and not at all on the organ-protective function of IL-22, or any antagonism of that function by IL-22BP. Consequently, the methods would not preclude the public from all uses of IL-22BP. Also apparent is that methods disclosed herein are based, at least in part, on the role of IFN-γ in immune responses, including autoimmune responses, and not at all on the role of IFN-γ in defending against infection. Thus, the methods would not preclude the public from all uses of IFN-γ.

In some embodiments of this aspect of the disclosure, the anti-IL-23 agent is Brazikumab comprising six complementarity determining regions specifically binding IL-23, i.e., HCDR1 of SEQ ID NO: 91, HCDR2 of SEQ ID NO: 92, HCDR3 of SEQ ID NO: 93, LCDR1 of SEQ ID NO: 62, LCDR2 of SEQ ID NO: 63, and LCDR3 of SEQ ID NO:64. In some embodiments, Brazikumab comprises the V_{H} sequence of SEQ ID NO:153 and the V_{L} sequence of SEQ ID NO:154. In some embodiments, Brazikumab comprises the V_{H} sequence of SEQ ID NO: 153 fused to a heavy chain constant region and the V_{L} sequence of SEQ ID NO: 154 fused to a light chain constant region. In some embodiments, the inflammatory condition is an inflammatory bowel disease, psoriasis, psoriatic arthritis, rheumatoid arthritis, or ankylosing spondylitis. Some embodiments are provided wherein the inflammatory bowel disease is Crohn's disease or ulcerative colitis. In some embodiments, the inflammatory condition is refractory to Tumor Necrosis Factor (TNF) treatment. In some embodiments, the serum level of Interleukin-22 Binding Protein is less than 359 pg/mL. The disclosure further contemplates embodiments of disclosed methods wherein the serum level of IL-22BP in Brazikumab non-responders is at least 359 pg/mL or is between 359 pg/mL to 6,000 pg/mL. In some embodiments, the level of IL-22BP in Brazikumab non-responders is 359-5,000 pg/mL, 359-4,000 pg/mL, 359-2,500 pg/mL, 359-1,000 pg/mL, 359-500 pg/mL, 400-6,000 pg/mL, 400-5,000 pg/mL, 400-4,000 pg/mL, 400-2,500 pg/mL, 400-1,000 pg/mL, 400-500 pg/mL 500-6,000 pg/mL, 500-5,000 pg/mL, 500-4,000 pg/mL, 500-2,500 pg/mL, 500-1,000 pg/mL, 750-6,000 pg/mL, 750-5,000 pg/mL, 750-4,000 pg/mL, 750-2,500 pg/mL, 750-1,000 pg/mL, 1,000-6,000 pg/mL, 1,000-5,000 pg/mL, 1,000-4,000 pg/mL, 1,000-2,500 pg/mL, 1,500-6,000 pg/mL, 1,500-5,000 pg/mL, 1,500-4,000 pg/mL, 1,500-2,500 pg/mL, 2,000-6,000 pg/mL, 2,000-5,000 pg/mL, or 2,000-2,500 pg/mL. In some embodiments, the method further comprises determining that a subject has an inflammatory condition, wherein the inflammatory condition is determined by conducting a physical examination, by consulting a subject's medical record, or by consulting with a medical practitioner. In some embodiments, the method further comprises administering an anti-IL-23 agent in an amount effective to treat the inflammatory condition, such as wherein the anti-IL-23 agent is Brazikumab.

In some embodiments according to the foregoing aspect of the disclosure, the anti-IL-23 agent is administered in an amount sufficient to achieve a serum concentration of from 12.5 ng/ml to 1,000 ng/ml. In some embodiments, the anti-IL23 agent, e.g., a heterodimer-specific anti-IL-23 antibody, is given to achieve a serum concentration of at least 12.5 ng/ml, 25 ng/ml, 50 ng/ml, 60 ng/ml, 70 ng/ml, 75 ng/ml, 80 ng/ml, 85 ng/ml, 90 ng/ml, 95 ng/ml, 100 ng/ml, 150 ng/ml, 200 ng/ml, 500 ng/ml, or 990 ng/ml. In some embodiments, the treatment is given in an amount of the anti-IL-23 agent and at an interval of 15-54 mg every 0.5-1.5 months; 55-149 mg every 1.5-4.5 months; 150-299 mg every 4-8 months; 300-1100 mg every 8-14 months, or 300-1100 mg every 4-12 months. In some embodiments, the treatment is given in an amount of the anti-IL-23 agent and at an interval of 15-21 mg every 0.5-1.0 month, 55-70 mg every 1.5-3.0 months, 150-260 mg every 4-6 months, or 300-700 mg every 4-8 months. In some embodiments, the amount and interval are 21 mg every month; 70 mg every 3 months; 210 mg every 6 months; or 700 mg every 6 months. In some embodiments, an anti-IL-23 antibody is administered at 260 mg every 3 months or at 700 mg every 3 months. In some embodiments, 260 mg of the antibody is administered every 1 month or 700 mg is administered every 1 month. In some embodiments, the dosage of an anti-IL-23 agent administered to a subject is 70 mg, e.g., administering 70 mg, 140 mg, 219 mg, 420 mg, or 700 mg per dose.

A related aspect of the disclosure is drawn to a method of treating an interleukin-23 (IL-23)-mediated inflammatory condition in a patient comprising administering an effective amount of an anti-IL-23 agent to a patient if the patient is determined to have a serum level of IL-22BP that is lower than an IL-22BP level in a control sample, wherein the control sample is obtained from one or more individuals without an inflammatory condition. In some embodiments, the anti-IL-23 agent is Brazikumab. In some embodiments, the inflammatory condition is an inflammatory bowel disease, psoriasis, psoriatic arthritis, rheumatoid arthritis, or ankylosing spondylitis. In some embodiments, the inflammatory bowel disease is Crohn's disease or ulcerative colitis. In some embodiments, the inflammatory condition is refractory to Tumor Necrosis Factor (TNF) treatment. In some embodiments, the anti-IL-23 agent is administered in an amount sufficient to achieve a serum concentration of from 12.5 ng/ml to 1,000 ng/ml. In some embodiments, the treatment is given in an amount of the anti-IL-23 agent and at an interval of 15-54 mg every 0.5-1.5 months; 55-149 mg every 1.5-4.5 months; 150-299 mg every 4-8 months; 300-1100 mg every 8-14 months, or 300-1100 mg every 4-12 months. In some embodiments, the treatment is given in an amount of the anti-IL-23 agent and at an interval of 15-21 mg every 0.5-1.0 month, 55-70 mg every 1.5-3.0 months, 150-260 mg every 4-6 months, or 300-700 mg every 4-8 months. In some embodiments, the amount and interval are 21 mg every month; 70 mg every 3 months; 210 mg every 6 months; or 700 mg every 6 months. In some embodiments, an anti-IL-23 antibody is administered at 260 mg every 3 months or at 700 mg every 3 months. In some embodiments, 260 mg of the antibody is administered every 1 month or 700 mg is administered every 1 month. In some embodiments, the dosage of an anti-IL-23 agent administered to a subject is 70 mg, e.g., administering 70 mg, 140 mg, 219 mg, 420 mg, or 700 mg per dose.

Another aspect of the disclosure is drawn to a method of selecting at least one member of an inflammatory bowel disease (IBD) patient sub-population amenable to treatment with an anti-Interleukin-23 (anti-IL-23) agent from a population of patients with IBD, wherein the sub-population of patients have IBD refractory to Tumor Necrosis Factor (TNF) treatment, the sub-population of patients have IBD naïve to treatment therefor, and/or the sub-population of patients is intolerant to treatment with an anti-TNF agent, comprising: (a) measuring the serum level of Interleukin-22 Binding Protein (IL-22BP) in an IBD patient; (b) comparing the serum IL-22BP level in the IBD patient to a serum level of IL-22BP in a control, wherein the serum level of IL-22BP in a control is any one of the serum level of IL-22BP in an individual without IBD, the mean level of IL-22BP in a plurality of individuals without an IBD, or the mean value of IL-22BP in a plurality of individuals with an IBD; and (c) selecting the patient as having an IBD responsive to treatment with an anti-IL-23 agent if the serum level of IL-22BP is lower in the subject than in the control, and optionally, (d) administering an effective amount of an anti-IL-23 agent to the patient. In some embodiments, the anti-IL-23 agent is Brazikumab. In some embodiments, the member of an IBD patient sub-population is a Crohn's disease patient or an ulcerative colitis patient. In some embodiments, the serum level of IL-22BP in a control is the mean value of IL-22BP in a plurality of individuals with an IBD, and some of these embodiments are embodiments wherein the IBD is Crohn's disease or ulcerative colitis. In some embodiments, the population of patients with IBD is the population of patients having IBD refractory to TNF treatment. In some embodiments of this aspect of the disclosure, the method further comprises determining that a subject has an inflammatory bowel disease, wherein the inflammatory bowel disease is determined by conducting a physical examination, by consulting a subject's medical record, or by consulting with a medical practitioner. In some embodiments, the method further comprises administering an anti-IL-23 agent in an amount effective to treat the inflammatory bowel condition, such as wherein the anti-IL-23 agent is Brazikumab. In some embodiments, the anti-IL-23 agent is administered in an amount sufficient to achieve a serum concentration of from 12.5 ng/ml to 1,000 ng/ml. In some embodiments, the treatment is given in an amount of the anti-IL-23 agent and at an interval of 15-54 mg every 0.5-1.5 months; 55-149 mg every 1.5-4.5 months; 150-299 mg every 4-8 months; 300-1100 mg every 8-14 months, or 300-1100 mg every 4-12 months. In some embodiments, the treatment is given in an amount of the anti-IL-23 agent and at an interval of 15-21 mg every 0.5-1.0 month, 55-70 mg every 1.5-3.0 months, 150-260 mg every 4-6 months, or 300-700 mg every 4-8 months. In some embodiments, the amount and interval are 21 mg every month; 70 mg every 3 months; 210 mg every 6 months; or 700 mg every 6 months. In some embodiments, an anti-IL-23 antibody is administered at 260 mg every 3 months or at 700 mg every 3 months. In some embodiments, 260 mg of the antibody is administered every 1 month or 700 mg is administered every 1 month. In some embodiments, the dosage of an anti-IL-23 agent administered to a subject is 70-mg, e.g., administering 70 mg, 140 mg, 219 mg, 420 mg, or 700 mg per dose.

In a related aspect, the disclosure provides a method of treating a patient that is a member of an inflammatory bowel disease (IBD) patient sub-population amenable to treatment with an anti-Interleukin-23 (anti-IL-23) agent, wherein the sub-population of patients has IBD refractory to Tumor Necrosis Factor (TNF) treatment, the sub-population of patients have IBD naïve to treatment therefor, and/or the sub-population of patients is intolerant to treatment with an anti-TNF agent, comprising administering an effective amount of an anti-IL-23 agent if the serum level of IL-22BP is lower in the patient than in a control, wherein the control is any one of the serum level of IL-22BP in an individual without IBD, the mean level of IL-22BP in a plurality of individuals without an IBD, or the mean level of IL-22BP in a plurality of individuals with an IBD. In some embodiments, the anti-IL-23 agent is Brazikumab. In some embodiments, the patient has Crohn's disease or ulcerative colitis. In some embodiments, the anti-IL-23 agent is administered in an amount sufficient to achieve a serum concentration of from 12.5 ng/ml to 1,000 ng/ml. In some embodiments, the treatment is given in an amount of the anti-IL-23 agent and at an interval of 15-54 mg every 0.5-1.5 months; 55-149 mg every 1.5-4.5 months; 150-299 mg every 4-8 months; 300-1100 mg every 8-14 months, or 300-1100 mg every 4-12 months. In some embodiments, the treatment is given in an amount of the anti-IL-23 agent and at an interval of 15-21 mg every 0.5-1.0 month, 55-70 mg every 1.5-3.0 months, 150-260 mg every 4-6 months, or 300-700 mg every 4-8 months. In some embodiments, the amount and interval are 21 mg every month; 70 mg every 3 months; 210 mg every 6 months; or 700 mg every 6 months. In some embodiments, an anti-IL-23 antibody is administered at 260 mg every 3 months or at 700 mg every 3 months. In some embodiments, 260 mg of the antibody is administered every 1 month or 700 mg is administered every 1 month. In some embodiments, the dosage of an anti-IL-23 agent administered to a subject is 70-mg, e.g., administering 70 mg, 140 mg, 219 mg, 420 mg, or 700 mg per dose.

Yet another aspect according to the disclosure is directed to a method of selecting a subject with an inflammatory condition amenable to treatment with an anti-Interleukin-23 (anti-IL-23) agent from a patient population having an inflammatory condition refractory to Tumor Necrosis Factor treatment, comprising: (a) measuring the serum level of Interleukin-22 Binding Protein (IL-22BP) in a subject; (b) comparing the serum IL-22BP level in the subject to a serum level of IL-22BP in a control, wherein the control is one or more individuals without an inflammatory condition refractory to Tumor Necrosis Factor treatment; and (c) selecting the subject as having an inflammatory condition amenable to treatment with an anti-IL-23 agent if the serum level of IL-22BP is lower in the subject than in the control. In some embodiments, the anti-IL-23 agent is Brazikumab. Embodiments are also envisioned wherein the inflammatory condition is an inflammatory bowel disease, psoriasis, psoriatic arthritis, rheumatoid arthritis, or ankylosing spondylitis. In some embodiments, the inflammatory bowel disease is Crohn's disease or ulcerative colitis. In some embodiments, the subject selected as having an inflammatory condition amenable to treatment with an anti-IL-23 agent has a serum level of IL-22BP less than 359 pg/mL. In some embodiments of this aspect of the disclosure, the method further comprises determining that a subject has an inflammatory condition refractory to Tumor Necrosis Factor (TNF) treatment, wherein the inflammatory condition refractory to TNF treatment is determined by conducting a physical examination, by consulting a subject's medical record, or by consulting with a medical practitioner. In some embodiments, the method further comprises administering an anti-IL-23 agent in an amount effective to treat the inflammatory condition refractory to Tumor Necrosis Factor (TNF) treatment, such as wherein the anti-IL-23 agent is Brazikumab. In some embodiments, the anti-IL-23 agent is administered in an amount sufficient to achieve a serum concentration of from 12.5 ng/ml to 1,000 ng/ml. In some embodiments, the treatment is given in an amount of the anti-IL-23 agent and at an interval of 15-54 mg every 0.5-1.5 months; 55-149 mg every 1.5-4.5 months; 150-299 mg every 4-8 months; 300-1100 mg every 8-14 months, or 300-1100 mg every 4-12 months. In some embodiments, the treatment is given in an amount of the anti-IL-23 agent and at an interval of 15-21 mg every 0.5-1.0 month, 55-70 mg every 1.5-3.0 months, 150-260 mg every 4-6 months, or 300-700 mg every 4-8 months. In some embodiments, the amount and interval are 21 mg every month; 70 mg every 3 months; 210 mg every 6 months; or 700 mg every 6 months. In some embodiments, an anti-IL-23 antibody is administered at 260 mg every 3 months or at 700 mg every 3 months. In some embodiments, 260 mg of the antibody is administered every 1 month or 700 mg is administered every 1 month. In some embodiments, the dosage of an anti-IL-23 agent administered to a subject is 70-mg, e.g., administering 70 mg, 140 mg, 219 mg, 420 mg, or 700 mg per dose.

A related aspect of the disclosure is directed to a method of treating a subject with an inflammatory condition refractory to Tumor Necrosis Factor treatment comprising administering an effective amount of an anti-IL-23 agent if the serum level of IL-22BP is lower in the subject than in a control, wherein the control is one or more individuals without an inflammatory condition refractory to Tumor Necrosis Factor treatment. In some embodiments, the anti-IL-23 agent is Brazikumab. In some embodiments, the inflammatory condition is an inflammatory bowel disease, psoriasis, psoriatic arthritis, rheumatoid arthritis, or ankylosing spondylitis. In some embodiments, the inflammatory bowel disease is Crohn's disease or ulcerative colitis. In some embodiments, the subject has a serum level of IL-22BP less than 359 pg/mL. In some embodiments, the anti-IL-23 agent is administered in an amount sufficient to achieve a serum concentration of from 12.5 ng/ml to 1,000 ng/ml. In some embodiments, the treatment is given in an amount of the anti-IL-23 agent and at an interval of 15-54 mg every 0.5-1.5 months; 55-149 mg every 1.5-4.5 months; 150-299 mg every 4-8 months; 300-1100 mg every 8-14 months, or 300-1100 mg every 4-12 months. In some embodiments, the treatment is given in an amount of the anti-IL-23 agent and at an interval of 15-21 mg every 0.5-1.0 month, 55-70 mg every 1.5-3.0 months, 150-260 mg every 4-6 months, or 300-700 mg every 4-8 months. In some embodiments, the amount and interval are 21 mg every month; 70 mg every 3 months; 210 mg every 6 months; or 700 mg every 6 months. In some embodiments, an anti-IL-23 antibody is administered at 260 mg every 3 months or at 700 mg every 3 months. In some embodiments, 260 mg of the antibody is administered every 1 month or 700 mg is administered every 1 month. In some embodiments, the dosage of an anti-IL-23 agent administered to a subject is 70-mg, e.g., administering 70 mg, 140 mg, 219 mg, 420 mg, or 700 mg per dose.

Still another aspect of the disclosure provides a method of selecting a subject with an inflammatory condition amenable to treatment with an anti-Interleukin-23 (anti-IL-23) agent, comprising: (a) measuring the serum level of Interferon-γ (IFN-γ) in a subject; (b) comparing the serum level of IFN-γ in the subject to a serum level of IFN-γ in a control, wherein the control is one or more individuals without an inflammatory condition; and (c) selecting the subject as having an inflammatory condition amenable to treatment with an anti-IL-23 agent if the serum level of IFN-γ is higher in the subject than in the control. In some embodiments, the anti-IL-23 agent is Brazikumab. In some embodiments, the inflammatory condition is an inflammatory bowel disease, psoriasis, psoriatic arthritis, rheumatoid arthritis, or ankylosing spondylitis. In some embodiments, the inflammatory bowel disease is Crohn's disease or ulcerative colitis. In some embodiments, the selected subject has a serum concentration of Interferon-γ that is greater than 15 pg/mL. In some embodiments, the inflammatory condition is refractory to Tumor Necrosis Factor treatment. In some embodiments of this aspect of the disclosure, the method further comprises determining that a subject has an inflammatory condition, wherein the inflammatory condition is determined by conducting a physical examination, by consulting a subject's medical record, or by consulting with a medical practitioner. In some embodiments, the method further comprises administering an anti-IL-23 agent in an amount effective to treat the inflammatory condition. In some embodiments, the anti-IL-23 agent is Brazikumab. In some embodiments, the anti-IL-23 agent is administered in an amount sufficient to achieve a serum concentration of from 12.5 ng/ml to 1,000 ng/ml. In some embodiments, the treatment is given in an amount of the anti-IL-23 agent and at an interval of 15-54 mg every 0.5-1.5 months; 55-149 mg every 1.5-4.5 months; 150-299 mg every 4-8 months; 300-1100 mg every 8-14 months, or 300-1100 mg every 4-12 months. In some embodiments, the treatment is given in an amount of the anti-IL-23 agent and at an interval of 15-21 mg every 0.5-1.0 month, 55-70 mg every 1.5-3.0 months, 150-260 mg every 4-6 months, or 300-700 mg every 4-8 months. In some embodiments, the amount and interval are 21 mg every month; 70 mg every 3 months; 210 mg every 6 months; or 700 mg every 6 months. In some embodiments, an anti-IL-23 antibody is administered at 260 mg every 3 months or at 700 mg every 3 months. In some embodiments, 260 mg of the antibody is administered every 1 month or 700 mg is administered every 1 month. In some embodiments, the dosage of an anti-IL-23 agent administered to a subject is 70-mg, e.g., administering 70 mg, 140 mg, 219 mg, 420 mg, or 700 mg per dose.

In a related aspect, the disclosure provides a method of treating a subject with an inflammatory condition comprising administering an effective amount of an anti-IL-23 agent to the subject if the serum level of Interferon-γ is higher in the subject than in a control, wherein the control is one or more individuals without an inflammatory condition. In some embodiments, the anti-IL-23 agent is Brazikumab. In some embodiments, the inflammatory condition is an inflammatory bowel disease, psoriasis, psoriatic arthritis, rheumatoid arthritis, or ankylosing spondylitis. In some embodiments, the inflammatory bowel disease is Crohn's disease or ulcerative colitis. In some embodiments, the inflammatory condition is refractory to Tumor Necrosis Factor treatment. In some embodiments, the selected subject has a serum concentration of Interferon-γ that is greater than 15 pg/mL. In some embodiments, the anti-IL-23 agent is administered in an amount sufficient to achieve a serum concentration of from 12.5 ng/ml to 1,000 ng/ml. In some embodiments, the treatment is given in an amount of the anti-IL-23 agent and at an interval of 15-54 mg every 0.5-1.5 months; 55-149 mg every 1.5-4.5 months; 150-299 mg every 4-8 months; 300-1100 mg every 8-14 months, or 300-1100 mg every 4-12 months. In some embodiments, the treatment is given in an amount of the anti-IL-23 agent and at an interval of 15-21 mg every 0.5-1.0 month, 55-70 mg every 1.5-3.0 months, 150-260 mg every 4-6 months, or 300-700 mg every 4-8 months. In some embodiments, the amount and interval are 21 mg every month; 70 mg every 3 months; 210 mg every 6 months; or 700 mg every 6 months. In some embodiments, an anti-IL-23 antibody is administered at 260 mg every 3 months or at 700 mg every 3 months. In some embodiments, 260 mg of the antibody is administered every 1 month or 700 mg is administered every 1 month. In some embodiments, the dosage of an anti-IL-23 agent administered to a subject is 70-mg, e.g., administering 70 mg, 140 mg, 219 mg, 420 mg, or 700 mg per dose.

Another aspect of the disclosure is drawn to a method of selecting at least one member of an inflammatory bowel disease (IBD) patient sub-population amenable to treatment with an anti-Interleukin-23 (anti-IL-23) agent from a population of patients with IBD, wherein the sub-population of patients have IBD refractory to Tumor Necrosis Factor (TNF) treatment, the sub-population of patients have IBD naïve to treatment therefor, and/or the sub-population of patients is intolerant to treatment with an anti-TNF agent, comprising: (a) measuring the serum level of Interferon-γ (IFN-γ) in an IBD patient; (b) comparing the serum IFN-γ level in the IBD patient to a serum level of IFN-γ in a control, wherein the serum level of IFN-γ in a control is any one of the serum level of IFN-γ in an individual without an IBD, the mean level of IFN-γ in a plurality of individuals without an IBD, or the mean value of IFN-γ in a plurality of individuals with an IBD; and (c) selecting the patient as having an IBD amenable to treatment with an anti-IL-23 agent if the serum level of IFN-γ is higher in the subject than in the control. In some embodiments, the anti-IL-23 agent is Brazikumab. In some embodiments, the member of an IBD patient sub-population is a Crohn's disease patient or an ulcerative colitis patient. In some embodiments, the serum level of IFN-γ in a control is the mean value of IFN-γ in a plurality of individuals with an IBD. In some embodiments, the IBD in a control is Crohn's disease or ulcerative colitis. In some embodiments, the population of patients with IBD is the population of patients having IBD refractory to TNF treatment. In some embodiments, the selected subject has a serum concentration of Interferon-γ that is greater than 15 pg/mL. In some embodiments of the method according to this aspect of the disclosure, the method further comprises determining that a subject has an inflammatory bowel disease, wherein the inflammatory bowel disease is determined by conducting a physical examination, by consulting a subject's medical record, or by consulting with a medical practitioner. In some embodiments, the method further comprises administering an anti-IL-23 agent in an amount effective to treat the inflammatory bowel condition. In some embodiments, the anti-IL-23 agent is Brazikumab. In some embodiments, the anti-IL-23 agent is administered in an amount sufficient to achieve a serum concentration of from 12.5 ng/ml to 1,000 ng/ml. In some embodiments, the treatment is given in an amount of the anti-IL-23 agent and at an interval of 15-54 mg every 0.5-1.5 months; 55-149 mg every 1.5-4.5 months; 150-299 mg every 4-8 months; 300-1100 mg every 8-14 months, or 300-1100 mg every 4-12 months. In some embodiments, the treatment is given in an amount of the anti-IL-23 agent and at an interval of 15-21 mg every 0.5-1.0 month, 55-70 mg every 1.5-3.0 months, 150-260 mg every 4-6 months, or 300-700 mg every 4-8 months. In some embodiments, the amount and interval are 21 mg every month; 70 mg every 3 months; 210 mg every 6 months; or 700 mg every 6 months. In some embodiments, an anti-IL-23 antibody is administered at 260 mg every 3 months or at 700 mg every 3 months. In some embodiments, 260 mg of the antibody is administered every 1 month or 700 mg is administered every 1 month. In some embodiments, the dosage of an anti-IL-23 agent administered to a subject is 70-mg, e.g., administering 70 mg, 140 mg, 219 mg, 420 mg, or 700 mg per dose.

A related aspect is drawn to a method of treating a patient that is a member of an inflammatory bowel disease (IBD) patient sub-population amenable to treatment with an anti-Interleukin-23 (anti-IL-23) agent, wherein the sub-population of patients have IBD refractory to Tumor Necrosis Factor treatment, the sub-population of patients have IBD naïve to treatment therefor, and/or the sub-population of patients is intolerant to treatment with an anti-TNF agent, comprising administering an effective amount of an anti-IL-23 agent if the serum level of Interferon-γ (IFN-γ) is higher in the patient than in a control, wherein the control is any one of the serum level of IFN-γ in an individual without an IBD, the mean level of IFN-γ in a plurality of individuals without an IBD, or the mean level of IFN-γ in a plurality of individuals with an IBD. In some embodiments, the anti-IL-23 agent is Brazikumab. In some embodiments, the inflammatory bowel disease is Crohn's disease or ulcerative colitis. In some embodiments, the selected subject has a serum concentration of Interferon-γ that is greater than 15 pg/mL. In some embodiments, the anti-IL-23 agent is administered in an amount sufficient to achieve a serum concentration of from 12.5 ng/ml to 1,000 ng/ml. In some embodiments, the treatment is given in an amount of the anti-IL-23 agent and at an interval of 15-54 mg every 0.5-1.5 months; 55-149 mg every 1.5-4.5 months; 150-299 mg every 4-8 months; 300-1100 mg every 8-14 months, or 300-1100 mg every 4-12 months. In some embodiments, the treatment is given in an amount of the anti-IL-23 agent and at an interval of 15-21 mg every 0.5-1.0 month, 55-70 mg every 1.5-3.0 months, 150-260 mg every 4-6 months, or 300-700 mg every 4-8 months. In some embodiments, the amount and interval are 21 mg every month; 70 mg every 3 months; 210 mg every 6 months; or 700 mg every 6 months. In some embodiments, an anti-IL-23 antibody is administered at 260 mg every 3 months or at 700 mg every 3 months. In some embodiments, 260 mg of the antibody is administered every 1 month or 700 mg is administered every 1 month. In some embodiments, the dosage of an anti-IL-23 agent administered to a subject is 70-mg, e.g., administering 70 mg, 140 mg, 219 mg, 420 mg, or 700 mg per dose.

Yet another aspect of the disclosure is directed to a method of selecting a subject with an inflammatory condition amenable to treatment with an anti-Interleukin-23 (anti-IL-23) agent from a patient population having an inflammatory condition refractory to Tumor Necrosis Factor (TNF) treatment, comprising: (a) measuring the serum level of Interferon-γ (IFN-γ) in a subject; (b) comparing the serum IFN-γ level in the subject to a serum level of IFN-γ in a control, wherein the control is one or more individuals without an inflammatory condition; and (c) selecting the subject as having an inflammatory condition amenable to treatment with an anti-IL-23 agent if the serum level of IFN-γ is higher in the subject than in the control. In some embodiments, the anti-IL-23 agent is Brazikumab. In some embodiments, the inflammatory condition is an inflammatory bowel disease, psoriasis, psoriatic arthritis, rheumatoid arthritis, or ankylosing spondylitis. In some embodiments, the inflammatory bowel disease is Crohn's disease or ulcerative colitis. In some embodiments, the selected subject has a serum concentration of Interferon-γ that is greater than 15 pg/mL. In some embodiments of this aspect of the disclosure, the method further comprises determining that a subject has an inflammatory condition refractory to Tumor Necrosis Factor (TNF) treatment, wherein the inflammatory condition refractory to Tumor Necrosis Factor (TNF) treatment is determined by conducting a physical examination, by consulting a subject's medical record, or by consulting with a medical practitioner. In some embodiments, the method further comprises administering an anti-IL-23 agent in an amount effective to treat the inflammatory condition refractory to Tumor Necrosis Factor (TNF) treatment. In some embodiments, the anti-IL-23 agent is Brazikumab. In some embodiments, the anti-IL-23 agent is administered in an amount sufficient to achieve a serum concentration of from 12.5 ng/ml to 1,000 ng/ml. In some embodiments, the treatment is given in an amount of the anti-IL-23 agent and at an interval of 15-54 mg every 0.5-1.5 months; 55-149 mg every 1.5-4.5 months; 150-299 mg every 4-8 months; 300-1100 mg every 8-14 months, or 300-1100 mg every 4-12 months. In some embodiments, the treatment is given in an amount of the anti-IL-23 agent and at an interval of 15-21 mg every 0.5-1.0 month, 55-70 mg every 1.5-3.0 months, 150-260 mg every 4-6 months, or 300-700 mg every 4-8 months. In some embodiments, the amount and interval are 21 mg every month; 70 mg every 3 months; 210 mg every 6 months; or 700 mg every 6 months. In some embodiments, an anti-IL-23 antibody is administered at 260 mg every 3 months or at 700 mg every 3 months. In some embodiments, 260 mg of the antibody is administered every 1 month or 700 mg is administered every 1 month. In some embodiments, the dosage of an anti-IL-23 agent administered to a subject is 70-mg, e.g., administering 70 mg, 140 mg, 219 mg, 420 mg, or 700 mg per dose.

Another aspect according to the disclosure focuses on a method of treating a subject with an inflammatory condition amenable to treatment with an anti-Interleukin-23 (anti-IL-23) agent, wherein the subject is a member of a patient population having an inflammatory condition refractory to Tumor Necrosis Factor treatment, comprising administering an effective amount of an anti-IL-23 agent if the serum level of IFN-γ is higher in the subject than in a control, wherein the control is one or more individuals without an inflammatory condition. In some embodiments, the anti-IL-23 agent is Brazikumab. In some embodiments, the inflammatory condition is an inflammatory bowel disease, psoriasis, psoriatic arthritis, rheumatoid arthritis, or ankylosing spondylitis. In some embodiments, the inflammatory bowel disease is Crohn's disease or ulcerative colitis. In some embodiments, the selected subject has a serum concentration of Interferon-γ that is greater than 15 pg/mL. In some embodiments, the anti-IL-23 agent is administered in an amount sufficient to achieve a serum concentration of from 12.5 ng/ml to 1,000 ng/ml. In some embodiments, the treatment is given in an amount of the anti-IL-23 agent and at an interval of 15-54 mg every 0.5-1.5 months; 55-149 mg every 1.5-4.5 months; 150-299 mg every 4-8 months; 300-1100 mg every 8-14 months, or 300-1100 mg every 4-12 months. In some embodiments, the treatment is given in an amount of the anti-IL-23 agent and at an interval of 15-21 mg every 0.5-1.0 month, 55-70 mg every 1.5-3.0 months, 150-260 mg every 4-6 months, or 300-700 mg every 4-8 months. In some embodiments, the amount and interval are 21 mg every month; 70 mg every 3 months; 210 mg every 6 months; or 700 mg every 6 months. In some embodiments, an anti-IL-23 antibody is administered at 260 mg every 3 months or at 700 mg every 3 months. In some embodiments, 260 mg of the antibody is administered every 1 month or 700 mg is administered every 1 month. In some embodiments, the dosage of an anti-IL-23 agent administered to a subject is 70-mg, e.g., administering 70 mg, 140 mg, 219 mg, 420 mg, or 700 mg per dose.

Another aspect of the disclosure is drawn to a method of selecting a subject with an inflammatory condition amenable to treatment with an anti-Interleukin-23 (anti-IL-23) agent, comprising: (a) measuring the serum level of Interleukin-22 Binding Protein (IL-22BP), Interferon-γ (IFN-γ), or both IL-22BP and IFN-γ in a subject; (b) comparing the serum level of IL-22BP, IFN-γ, or both IL-22BP and IFN-γ in the subject to a serum level of IL-22BP, IFN-γ, or both IL-22BP and IFN-γ in a control, wherein the control is one or more individuals without an inflammatory condition; and (c) selecting the subject as having an inflammatory condition amenable to treatment with an anti-IL-23 agent if the serum level of IL-22BP is lower, the serum level of IFN-γ is higher, or both the serum level of IL-22BP is lower and the serum level of IFN-γ is higher in the subject than in the control. In some of these embodiments, the anti-IL-23 agent is Brazikumab. In some of these embodiments, the inflammatory condition is an inflammatory bowel disease, psoriasis, psoriatic arthritis, rheumatoid arthritis, or ankylosing spondylitis. In some of these embodiments, the inflammatory bowel disease is Crohn's disease or ulcerative colitis. In some embodiments, the selected subject has a serum concentration of Interferon-γ that is greater than 15 pg/mL. In some of these embodiments, the method further comprises determining that a subject has an inflammatory condition refractory to Tumor Necrosis Factor (TNF) treatment, wherein the inflammatory condition refractory to Tumor Necrosis Factor (TNF) treatment is determined by conducting a physical examination, by consulting a subject's medical record, or by consulting with a medical practitioner. In some of these embodiments, the method further comprises administering an anti-IL-23 agent in an amount effective to treat the inflammatory condition refractory to Tumor Necrosis Factor (TNF) treatment. In some of these embodiments, the anti-IL-23 agent is Brazikumab. In some embodiments, the method further comprises administering an effective amount of an anti-IL-23 agent to the patient if the serum level of IFN-γ higher, and optionally the serum level of IL-22BP is lower, in the patient than in the control. In some embodiments, the anti-IL-23 agent is Brazikumab. In some embodiments, the inflammatory condition is an inflammatory bowel disease, psoriasis, psoriatic arthritis, rheumatoid arthritis, or ankylosing spondylitis. In some embodiments, the inflammatory bowel disease is Crohn's disease or ulcerative colitis.

Other features and advantages of the disclosure will be better understood by reference to the following detailed description, including the examples.

### BRIEF DESCRIPTION OF THE DRAWING

FIG. 1. Raw optical density (OD) readings of ELISA plates using Softmax software on an M5 ELISA plate reader (Molecular Devices, Inc., San Jose Calif.). Panel A provides the plate layout, panel B provides the raw OD values, and panel C provides adjusted values for the assays. A1-G1 and A2-G2 are technical duplicates of a human IL-22 BP standard curve. A3-A11, B3-B111, C3-C11, D3-D11, E3-E11, F3-F11, G3-G10 and H3-H10 are raw OD readings of healthy men, healthy women, Crohn's disease patients, Ulcerative colitis patients and Medimmune Phase 2a clinical trial (MED12070-1147) placebo group serum samples. The raw OD readings were converted to IL-22BP serum concentrations in the following FIGS. 2-8 using Softmax software.

FIG. 2. An IL-22BP standard curve consists of 7 calibrator concentrations ranging from 100-6000 pg/ml. The precision (CV %) and accuracy (% of recovery) of this ELISA are within the accepted ranges (CV %≤20% and Recovery % within 80-120%), demonstrating the validity of this analytical run.

FIG. 3. IL-22 BP serum levels from 10 healthy men were calculated based on the standard curve presented in FIG. 8 with standard curve data presented in FIG. 2.

FIG. 4. IL-22 BP serum levels from 10 healthy women were calculated based on the standard curve presented in FIG. 8 with standard curve data presented in FIG. 2.

FIG. 5. IL-22 BP serum levels from 19 Crohn's Disease patients were calculated based on the standard curve presented in FIG. 8 with standard curve data presented in FIG. 2.

FIG. 6. IL-22 BP serum levels from 11 Ulcerative Colitis patients were calculated based on the standard curve presented in FIG. 8 with standard curve data presented in FIG. 2.

FIG. 7. IL-22 BP serum levels from 20 Crohn's Disease patients (non-responders to anti-TNFα treatment) in Medimmune Phase 2a trial (MED12070-1147) placebo group serum samples were calculated based on the standard curve presented in FIG. 8 with standard curve data presented in FIG. 2.

FIG. 8. IL-22 BP standard curve graph generated using Softmax software. IL-22 BP concentrations in pg/mL

FIG. 9. Summary of results from FIGS. 3-7. Data revealed that both median and average serum concentrations of IL-22 BP in Crohn's Disease (CD) and Ulcerative Colitis patients were higher than those of healthy, or normal, people (men and women). Also, levels of IL-22BP in anti-TNFα non-responder CD patients were lower than that of healthy humans. Expressed IL-22BP levels in serum are expected to exhibit a polarized pattern in Brazikumab responder versus non-responder subpopulations, regardless of whether the Brazikumab responder and non-responder subpopulations are refractory to TNF-α treatment.

FIG. 10. Serum IFN-γ levels in anti-TNF-α refractory Crohn's Disease patients. Serum samples were obtained from the group of patients treated with Brazikumab in a Medimmune phase 2a trial (MED12070-1147). Responders to Brazikumab in the clinical trial are identified with a “0” in the “Responder” column in panel A; non-responders to Brazikumab in the clinical trial are identified with a “1” in that column as shown in panel B.

FIG. 11. ELISA results revealing that both median and average serum concentrations of IL-22 BP in the Brazikumab responder subpopulation of Crohn's disease patients (see panel B) were lower than those of Brazikumab non-responder CD patients (see panel A). Serum samples were obtained from the group of CD patients treated with Brazikumab in a Medimmune phase 2a trial (MED12070-1147). All patients included in this phase 2a trial were non-responders to anti-TNFα treatment.

### DETAILED DESCRIPTION

The disclosure provides methods and materials for selecting patient populations, or sub-populations, amenable to anti-cytokine therapy, and more particularly anti-IL-23 therapies including anti-IL-23 immunotherapies, to treat inflammatory conditions. Based on the experimental data disclosed herein, and contrary to the conventional belief that IL-22BP would be elevated in patients responsive to anti-IL-23 therapy, methods of selecting patients amenable to anti-IL-23 therapy to treat inflammatory conditions are provided that determine if a serum sample of a patient show a reduced level of Interleukin-22 Binding Protein (IL-22BP) and/or an elevated level of Interferon-γ (IFN-γ).

The disclosure further provides IL-23 antigen-binding proteins, including molecules that antagonize IL-23, such as anti-IL-23 antibodies, antibody fragments, and antibody derivatives, e.g., antagonistic anti-IL-23 antibodies, antibody fragments, or antibody derivatives. Also provided are polynucleotides, and derivatives and fragments thereof, comprising a sequence of nucleic acids that encodes all or a portion of a polypeptide that binds to IL-23, e.g., a polynucleotide encoding all or part of an anti-IL-23 antibody, antibody fragment, or antibody derivative, plasmids and vectors comprising such nucleic acids, and cells or cell lines comprising such polynucleotides and/or vectors and plasmids. The provided methods include, for example, methods of making, identifying, or isolating IL-23 antigen-binding proteins, such as anti-IL-23 antibodies, methods of determining whether a molecule binds to IL-23, methods of determining whether a molecule antagonizes IL-23, methods of making compositions, such as pharmaceutical compositions, comprising an IL-23 antigen-binding protein, and methods for administering an IL-23 antigen-binding protein to a subject, for example, methods for treating a condition mediated by IL-23, and for antagonizing a biological activity of IL-23, in vivo or in vitro.

Unless otherwise defined herein, scientific and technical terms used in connection with the disclosure shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated. See, e.g., Sambrook et al., Molecular Cloning: A Laboratory Manual, 3rd ed., Cold Spring Harbor Laboratory Press, Cold Spring Harbor, N.Y. (2001) and Ausubel et al., Current Protocols in Molecular Biology, Greene Publishing Associates (1992), and Harlow and Lane, Antibodies: A Laboratory Manual Cold Spring Harbor Laboratory Press, Cold Spring Harbor, N.Y. (1990). Enzymatic reactions and purification techniques are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are those well-known and commonly used in the art. Standard techniques can be used for chemical syntheses, chemical analyses, pharmaceutical preparation, formulation, and delivery, and treatment of patients.

The polynucleotide and protein sequences of the p19 subunit of human IL-23 (SEQ ID NOs:144 and 145), the shared p40 subunit (SEQ ID NOs:146 and 147), the human IL-23 receptor heterodimeric subunits IL-12Rs1 (SEQ ID NOs:150 and 151) and IL-23R (SEQ ID NOs:148 and 149), are known in the art. See, for example, GenBank Accession Nos. AB030000; M65272, NM 005535, NM 144701, as are those from other mammalian species. Recombinant IL-23 and IL-23 receptor proteins including single chain and Fc proteins, as well as cells expressing the IL-23 receptor have been described or are available from commercial sources (see, for example, Oppmann et al., Immunity, 2000, 13: 713-715; R&D Systems, Minneapolis, Minn.; United States Biological, Swampscott, Mass.; WIPO Publication No. WO 2007/076524). Native human IL-23 can be obtained from human cells such as dendritic cells using methods known in the art, including those described herein.

IL-23 is a heterodimeric cytokine comprised of a unique p19 subunit that is covalently bound to a shared p40 subunit. The p19 subunit comprises four α-helices, “A”, “B”, “C” and “D” in an up-up-down-down motif joined by three intra-helix loops between the A and B helices, between the B and C helices and between the C and D helices, see Oppmann et al., Immunity, 2000, 13: 713-715 and Beyer, et al., J Mol Biol, 2008. 382(4): 942-55. The A and D helices of 4 helical bundle cytokines are believed to be involved with receptor binding. The p40 subunit comprises three beta-sheet sandwich domains, D1, D2 and D3 (Lupardus and Garcia, J. Mol. Biol., 2008, 382:931-941).

The term “polynucleotide” includes both single-stranded and double-stranded nucleic acids and includes genomic DNA, RNA, mRNA, cDNA, or synthetic origin or some combination thereof that is not associated with sequences normally found in nature. Isolated polynucleotides comprising specified sequences may include, in addition to the specified sequences, coding sequences for up to ten or even up to twenty other proteins or portions thereof, or may include operably linked regulatory sequences that control expression of the coding region of the recited nucleic acid sequences, and/or may include vector sequences. The nucleotides comprising the polynucleotide can be ribonucleotides or deoxyribonucleotides or a modified form of either type of nucleotide. The modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phosphoraniladate and phosphoroamidate.

The term “oligonucleotide” means a polynucleotide comprising 100 or fewer nucleotides. In some embodiments, oligonucleotides are 10 to 60 bases in length. In other embodiments, oligonucleotides are 12, 13, 14, 15, 16, 17, 18, 19, or 20 to 40 nucleotides in length. Oligonucleotides may be single-stranded or double-stranded, e.g., for use in the construction of a mutant gene. Oligonucleotides may be sense or antisense oligonucleotides. An oligonucleotide can include a detectable label, such as a radiolabel, a fluorescent label, a hapten or an antigenic label, for detection assays. Oligonucleotides may be used, for example, as PCR primers, cloning primers or hybridization probes.

The terms “polypeptide” or “protein” means a macromolecule having the amino acid sequence of a native protein, that is, a protein produced by a naturally occurring and non-recombinant cell; or it is produced by a genetically engineered or recombinant cell, and comprises molecules having the amino acid sequence of the native protein, or molecules having one or more deletions from, insertions to, and/or substitutions of, the amino acid residues of the native sequence. The term also includes amino acid polymers in which one or more amino acids are chemical analogs of a corresponding naturally occurring amino acid and polymers. The terms “polypeptide” and “protein” encompass IL-23 antigen-binding proteins (such as antibodies) and sequences that have one or more deletions from, additions to, and/or substitutions of, the amino acid residues of the antigen-binding protein sequence. The term “polypeptide fragment” refers to a polypeptide that has an amino-terminal deletion, a carboxyl-terminal deletion, and/or an internal deletion as compared with the full-length native protein. Such fragments may also contain modified amino acids as compared with the native protein. In certain embodiments, fragments are about five to 500 amino acids long. For example, fragments may be at least 5, 6, 7, 8, 9, 10, 11, 12, 13, 14, 15, 16, 17, 18, 19, 20, 50, 70, 100, 110, 150, 200, 250, 300, 350, 400, or 450 amino acids long. Useful polypeptide fragments include immunologically functional fragments of antibodies, including binding domains. In the case of an IL-23 antigen-binding protein, such as an antibody, useful fragments include, but are not limited to, one or more CDR regions, a variable domain of a heavy or light chain, a portion of an antibody chain, a portion of a variable region including less than three CDRs, and the like.

“Amino acid” is given its normal meaning in the art. The twenty naturally occurring amino acids and their abbreviations follow conventional usage. See, Immunology-A Synthesis, 2nd Edition, (E. S. Golub and D. R. Gren, eds.), Sinauer Associates: Sunderland, Mass. (1991). Stereoisomers (e.g., D-amino acids) of the twenty conventional amino acids, unnatural amino acids such as [alpha]-, [alpha]-disubstituted amino acids, N-alkyl amino acids, and other unconventional amino acids may also be suitable components for polypeptides. Examples of unconventional amino acids include: 4-hydroxyproline, [gamma]-carboxyglutamate, [epsilon]-N,N,N-trimethyllysine, [epsilon]-N-acetyllysine, 0-phosphoserine, N-acetylserine, N-formylmethionine, 3-methylhistidine, 5-hydroxylysine, [sigma]-N-methylarginine, and other similar amino acids and imino acids (e.g., 4-hydroxyproline). In the polypeptide notation used herein, the left-hand direction is the amino terminal direction and the right-hand direction is the carboxyl-terminal direction, in accordance with standard usage and convention.

The term “isolated protein” refers to a protein, such as an antigen-binding protein (an example of which could be an antibody), that is purified from proteins or polypeptides or other contaminants that would interfere with its therapeutic, diagnostic, prophylactic, research or other use. As used herein, “substantially pure” means that the described species of molecule is the predominant species present, that is, on a molar basis it is more abundant than any other individual species in the same mixture. In certain embodiments, a substantially pure molecule is a composition wherein the object species comprises at least 50% (on a molar basis) of all macromolecular species present. In other embodiments, a substantially pure composition will comprise at least 80%, 85%, 90%, 95%, or 99% of all macromolecular species present in the composition. In certain embodiments, an essentially homogeneous substance has been purified to such a degree that contaminating species cannot be detected in the composition by conventional detection methods and thus the composition consists of a single detectable macromolecular species.

A “variant” of a polypeptide (e.g., an antigen-binding protein such as an antibody) comprises an amino acid sequence wherein one or more amino acid residues are inserted into, deleted from, and/or substituted into, the amino acid sequence relative to another polypeptide sequence. Variants include fusion proteins. A “derivative” of a polypeptide is a polypeptide that has been chemically modified in some manner distinct from insertion, deletion, or substitution variants, e.g., via conjugation to another chemical moiety.

The terms “naturally occurring” or “native” as used throughout the specification in connection with biological materials such as polypeptides, nucleic acids, host cells, and the like, refers to materials which are found in nature, such as native human IL-23. In certain aspects, recombinant antigen-binding proteins that bind native IL-23 are provided. In this context, a “recombinant protein” is a protein made using recombinant techniques, i.e., through the expression of a recombinant nucleic acid as described herein. Methods and techniques for the production of recombinant proteins are well known in the art.

The term “antibody” refers to an intact immunoglobulin of any isotype, or a fragment thereof that can compete with the intact antibody for specific binding to the target antigen, and includes, for instance, chimeric, humanized, fully human, and bispecific antibodies. An antibody is a type of antigen-binding protein. Unless otherwise indicated, the term “antibody” includes, in addition to antibodies comprising two full-length heavy chains and two full-length light chains, derivatives, variants, fragments, and muteins thereof, examples of which are described below. An intact antibody generally will comprise at least two full-length heavy chains and two full-length light chains, but in some instances may include fewer chains such as antibodies naturally occurring in camelids which may comprise only heavy chains. Antibodies may be derived solely from a single source, or may be “chimeric,” that is, different portions of the antibody may be derived from two different antibodies, as described further below. The antigen-binding proteins, antibodies, or binding fragments may be produced in hybridomas, by recombinant DNA techniques, or by enzymatic or chemical cleavage of intact antibodies.

The term “functional fragment” (or simply “fragment”) of an antibody or immunoglobulin chain (heavy or light chain), as used herein, is an antigen-binding protein comprising a portion (regardless of how that portion is obtained or synthesized) of an antibody that lacks at least some of the amino acids present in a full-length chain but which is capable of specifically binding to an antigen. Such fragments are biologically active in that they bind specifically to the target antigen and can compete with other antigen-binding proteins, including intact antibodies, for specific binding to a given epitope. In one aspect, such a fragment will retain at least one CDR present in the full-length light or heavy chain, and in some embodiments will comprise a single heavy chain and/or light chain or portion thereof. These biologically active fragments may be produced by recombinant DNA techniques, or may be produced by enzymatic or chemical cleavage of antigen-binding proteins, including intact antibodies. Fragments include, but are not limited to, immunologically functional fragments such as Fab, Fab′, F(ab′)2, Fv, domain antibodies and single-chain antibodies, and may be derived from any mammalian source, including but not limited to human, mouse, rat, camelid or rabbit. It is contemplated further that a functional portion of the antigen-binding proteins disclosed herein, for example, one or more CDRs, could be covalently bound to a second protein or to a small molecule to create a therapeutic agent directed to a particular target in the body, possessing bifunctional therapeutic properties, or having a prolonged serum half-life.

The term “compete” when used in the context of antigen-binding proteins (e.g., neutralizing antigen-binding proteins or neutralizing antibodies) means competition between antigen-binding proteins as determined by an assay in which the antigen-binding protein (e.g., antibody or immunologically functional fragment thereof) under test prevents or inhibits specific binding of a reference antigen-binding protein (e.g., a ligand, or a reference antibody) to a common antigen (e.g., an IL-23 protein or a fragment thereof). Numerous types of competitive binding assays can be used, for example: solid-phase direct or indirect radioimmunoassay (RIA), solid-phase direct or indirect enzyme immunoassay (EIA), sandwich competition assay (see, e.g., Stahli et al., 1983, Methods in Enzymology 92:242-253); solid-phase direct biotin-avidin EIA (see, e.g., Kirkland et al., 1986, J. Immunol. 137:3614-3619) solid-phase direct-labeled assay, solid-phase direct-labeled sandwich assay (see, e.g., Harlow and Lane, 1988, Antibodies, A Laboratory Manual, Cold Spring Harbor Press); solid-phase direct-label RIA using ¹²⁵I label (see, e.g., Morel et al., 1988, Molec. Immunol. 25:7-15); solid-phase direct biotin-avidin EIA (see, e.g., Cheung, et al., 1990, Virology 176:546-552); and direct-labeled RIA (Moldenhauer et al., 1990, Scand. J. Immunol. 32:77-82). Typically, such an assay involves the use of purified antigen bound to a solid surface or cells bearing either of these, an unlabeled test antigen-binding protein and a labeled reference antigen-binding protein.

Competitive inhibition is measured by determining the amount of label bound to the solid surface or cells in the presence of the test antigen-binding protein. Usually the test antigen-binding protein is present in excess. Antigen-binding proteins identified by competition assay (competing antigen-binding proteins) include antigen-binding proteins binding to the same epitope as the reference antigen-binding proteins and antigen-binding proteins binding to an adjacent epitope sufficiently proximal to the epitope bound by the reference antigen-binding protein for steric hindrance to occur. Usually, when a competing antigen-binding protein is present in excess, it will inhibit specific binding of a reference antigen-binding protein to a common antigen by at least 40, 45%, 50%, 55%, 60%, 65%, 70% or 75%. In some instances, binding is inhibited by at least 80%, 85%, 90%, 91%, 92%, 93%, 94%, 95%, 96%, 97% 98%, 99% or more.

The term “epitope” or “antigenic determinant” refers to a site on an antigen to which an antigen-binding protein binds. Epitopes can be formed both from contiguous amino acids or non-contiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. Epitope determinants may include chemically active surface groupings of molecules, such as amino acids, sugar side chains, phosphoryl or sulfonyl groups, and may have specific three dimensional structural characteristics, and/or specific charge characteristics. An epitope typically includes at least 3, 4, 5, 6, 7, 8, 9, 10, 11, 12, 13, 14, 15, 16, 17, 18, 19, 20, 25, 30, 35 amino acids in a unique spatial conformation. Epitopes can be determined using methods known in the art.

### IL-23 Antigen-Binding Proteins

An “antigen-binding protein” as used herein means a protein that specifically binds a specified target antigen; the antigen as provided herein is IL-23, particularly human IL-23, including native human IL-23. Antigen-binding proteins as provided herein interact with at least a portion of the unique p19 subunit of IL-23, detectably binding IL-23; but do not bind with any significance to IL-12 (e.g., the p40 and/or the p35 subunits of IL-12), thus “sparing IL-12”. As a consequence, the antigen-binding proteins provided herein are capable of affecting IL-23 activity without the potential risks associated with inhibiting IL-12 or the p40 subunit shared by IL-12 and IL-23. The antigen-binding proteins may affect the ability of IL-23 to interact with its receptor, for example by affecting IL-23 binding to the receptor, such as by interfering with receptor association. In particular, such antigen-binding proteins totally or partially reduce, inhibit, interfere with, or modulate one or more biological activities of IL-23. Such inhibition or neutralization disrupts a biological response in the presence of the antigen-binding protein compared to the response in the absence of the antigen-binding protein and can be determined using assays known in the art and described herein. Antigen-binding proteins provided herein inhibit IL-23-induced pro-inflammatory cytokine production, for example IL-23-induced IL-22 production in whole blood cells and IL-23-induced IFN-γexpression in NK and whole blood cells. Reduction of biological activity can be about 20%, 30%, 40%, 50%, 60%, 70%, 80%, 85%, 90%, 91%, 92%, 93%, 94%, 95%, 96%, 97%, 98%, 99% or more.

An antigen-binding protein may comprise a portion that binds to an antigen and, optionally, a scaffold or framework portion that allows the antigen-binding portion to adopt a conformation that promotes binding of the antigen-binding protein to the antigen. Examples of antigen-binding proteins include antibodies, antibody fragments (e.g., an antigen-binding portion of an antibody), antibody derivatives, and antibody analogs. The antigen-binding protein can comprise an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen-binding protein as well as wholly synthetic scaffolds comprising, for example, a biocompatible polymer. See, for example, Korndorfer et al., Proteins: Structure, Function, and Bioinformatics, (2003) Volume 53, Issue 1:121-129; Roque et al., Biotechnol. Prog., 2004, 20:639-654. In addition, peptide antibody mimetics (“PAMs”) can be used, as well as scaffolds based on antibody mimetics utilizing fibronectin components as a scaffold.

Certain antigen-binding proteins described herein are antibodies or are derived from antibodies. Such antigen-binding proteins include, but are not limited to, monoclonal antibodies, bispecific antibodies, minibodies, domain antibodies, synthetic antibodies, antibody mimetics, chimeric antibodies, humanized antibodies, human antibodies, antibody fusions, antibody conjugates, single-chain antibodies, and fragments thereof, respectively. In some instances, the antigen-binding protein is an immunological fragment of an antibody (e.g., a Fab, a Fab′, a F(ab′)2, or a scFv). The various structures are further described and defined herein.

Certain antigen-binding proteins that are provided may comprise one or more CDRs as described herein (e.g., 1, 2, 3, 4, 5, 6 or more CDRs). In some instances, the antigen-binding protein comprises (a) a polypeptide structure and (b) one or more CDRs that are inserted into and/or joined to the polypeptide structure. The polypeptide structure can take a variety of different forms. For example, it can be, or comprise, the framework of a naturally occurring antibody, or fragment or variant thereof, or may be completely synthetic in nature. Examples of various polypeptide structures are further described below.

An antigen-binding protein of the disclosure is said to “specifically bind” its target antigen when the dissociation equilibrium constant (K_{D}) is less than or equal to 10⁻⁸ M. The antigen-binding protein specifically binds antigen with “high affinity” when the K_{D} is at least 5×10⁻⁹ M, and with “very high affinity” when the K_{D} is at least 5×10⁻¹⁰ M. In one embodiment the antigen-binding protein will bind to human IL-23 with a K_{D} of 5×10⁻¹² M, and in yet another embodiment it will bind with a K_{D} of 5×10⁻¹³ M. In another embodiment of the invention, the antigen-binding protein has a K_{D} of 5×10⁻¹² M and a K_{off} of about 5×10⁻⁻⁶ 1/s. In another embodiment, the K_{off} is 5×10⁻⁷ 1/s.

Another aspect provides an antigen-binding protein having a half-life of at least one day in vitro or in vivo (e.g., when administered to a human subject). In one embodiment, the antigen-binding protein has a half-life of at least three days. In another embodiment, the antibody or portion thereof has a half-life of four days or longer. In another embodiment, the antibody or portion thereof has a half-life of eight days or longer. In another embodiment, the antibody or antigen-binding portion thereof is derivatized or modified such that it has a longer half-life as compared to the underivatized or unmodified antibody. In another embodiment, the antigen-binding protein contains point mutations to increase serum half-life, such as described in WIPO Publication No. WO 00/09560.

In embodiments where the antigen-binding protein is used for therapeutic applications, an antigen-binding protein can reduce, inhibit, interfere with or modulate one or more biological activities of IL-23, such inducing production of proinflammatory cytokines. IL-23 has many distinct biological effects, which can be measured in many different assays in different cell types; examples of such assays and known and are provided herein.

Some of the antigen-binding proteins that are provided have the structure typically associated with naturally occurring antibodies. The structural units of these antibodies typically comprise one or more tetramers, each composed of two identical couplets of polypeptide chains, though some species of mammals also produce antibodies having only a single heavy chain. In a typical antibody, each pair or couplet includes one full-length “light” chain (in certain embodiments, about 25 kDa) and one full-length “heavy” chain (in certain embodiments, about 50-70 kDa). Each individual immunoglobulin chain is composed of several “immunoglobulin domains”, each consisting of roughly 90 to 110 amino acids and expressing a characteristic folding pattern. These domains are the basic units of which antibody polypeptides are composed. The amino-terminal portion of each chain typically includes a variable region that is responsible for antigen recognition. The carboxy-terminal portion is more conserved evolutionarily than the other end of the chain and is referred to as the “constant region” or “C region”. Human light chains generally are classified as kappa and lambda light chains, and each of these contains one variable region and one constant domain (CL1).z Heavy chains are typically classified as mu, delta, gamma, alpha, or epsilon chains, and these define the antibody's isotype as IgM, IgD, IgG, IgA, and IgE, respectively. IgG has several subtypes, including, but not limited to, IgG1, IgG2, IgG3, and IgG4. IgM subtypes include IgM, and IgM2. IgA subtypes include IgA1 and IgA2. In humans, the IgA and IgD isotypes contain four heavy chains and four light chains; the IgG and IgE isotypes contain two heavy chains and two light chains; and the IgM isotype contains five heavy chains and five light chains. The heavy chain constant region (CH) typically comprises one or more domains that may be responsible for effector function. The number of heavy chain constant region domains will depend on the isotype. IgG heavy chains, for example, each contains three CH region domains known as CH1, CH2 and CH3. The antibodies that are provided can have any of these isotypes and subtypes, for example, the IL-23 antigen-binding protein is of the IgG1, IgG2, or IgG4 subtype. If an IgG4 is desired, it may also be desired to introduce a point mutation (CPSCP->CPPCP) in the hinge region as described in Bloom et al., 1997, Protein Science 6:407) to alleviate a tendency to form intra-H chain disulfide bonds that can lead to heterogeneity in the IgG4 antibodies. Antibodies provided herein that are of one type can be changed to a different type using subclass switching methods. See, e.g., Lantto et al., 2002, Methods Mol. Biol. 178:303-316.

In full-length light and heavy chains, the variable and constant regions are joined by a “J” region of about twelve or more amino acids, with the heavy chain also including a “D” region of about ten more amino acids. See, e.g., Fundamental Immunology, 2nd ed., Ch. 7 (Paul, W., ed.) 1989, New York: Raven Press. The variable regions of each light/heavy chain pair typically form the antigen binding site.

### Variable Regions

Various heavy chain and light chain variable regions (or domains) provided herein are depicted in Tables 1 and 2. Each of these variable regions may be attached, for example, to heavy and light chain constant regions described above. Further, each of the so generated heavy and light chain sequences may be combined to form a complete antigen-binding protein structure.

Provided are antigen-binding proteins that contain at least one heavy chain variable region (VH) selected from the group consisting of VH1, VH2, VH3, VH4, VH5, VH6, VH7, VH8, VH9, VH10, VH11, VH12, VH13, VH14, VH15 and VH16 and/or at least one light chain variable region (VL) selected from the group consisting of VL1, VL2, VL3, VL4, VL5, VL6, VL7, VL8, VL9, VL10, VL11, VL12, VL13, VL14, VL15, and VL16 as shown in Tables 1 and 2 below.

Each of the heavy chain variable regions listed in Table 2 may be combined with any of the light chain variable regions shown in Table 1 to form an antigen-binding protein. In some instances, the antigen-binding protein includes at least one heavy chain variable region and/or one light chain variable region from those listed in Tables 1 and 2. In some instances, the antigen-binding protein includes at least two different heavy chain variable regions and/or light chain variable regions from those listed in Tables 1 and 2. The various combinations of heavy chain variable regions may be combined with any of the various combinations of light chain variable regions.

In other instances, the antigen-binding protein contains two identical light chain variable regions and/or two identical heavy chain variable regions. As an example, the antigen-binding protein may be an antibody or immunologically functional fragment that comprises two light chain variable regions and two heavy chain variable regions in combinations of pairs of light chain variable regions and pairs of heavy chain variable regions as listed in Tables 1 and 2. Examples of such antigen-binding proteins comprising two identical heavy chain and light chain variable regions include: Antibody A VH14/VL14; Antibody B VH9/VL9; Antibody C VH10/VL10; Antibody D VH15/VL15; Antibody E VH1/VL1, Antibody F VH11/VL11; Antibody G VH12/VL12; Antibody H VH13/VL13; Antibody I VH8/VL8; Antibody J VH3/VL3; Antibody K VH7/VL7; Antibody L VH4/VL4; Antibody M VH5/VL5 and Antibody N VH6/VL6.

Some antigen-binding proteins that are provided comprise a heavy chain variable region and/or a light chain variable region comprising a sequence of amino acids that differs from the sequence of a heavy chain variable region and/or a light chain variable region selected from Tables 1 and 2 at only 1, 2, 3, 4, 5, 6, 7, 8, 9, 10, 11, 12, 13, 14 or 15 amino acid residues, wherein each such sequence difference is independently either a deletion, insertion or substitution of one amino acid. The light and heavy chain variable regions, in some antigen-binding proteins, comprise sequences of amino acids that have at least 70%, 75%, 80%, 85%, 90%, 91%, 92%, 93%, 94%, 95%, 96%, 97%, 98% or 99% sequence identity to the amino acid sequences provided in Tables 1 and 2. Still other antigen-binding proteins, e.g., antibodies or immunologically functional fragments, also include variant heavy chain region forms and/or variant light chain region forms as described herein.

The term “identity” refers to a relationship between the sequences of two or more polypeptide molecules or two or more polynucleotides, as determined by aligning and comparing the sequences. “Percent identity” means the percent of identical residues between the amino acids or nucleotides in the compared molecules and is calculated based on the size of the smallest of the molecules being compared.

TABLE 1
Exemplary Variant Light Chain Region Sequences

| | FR1 CDRL1 FR2 CDRL2 FR3 CDRL3 FR4 |
|---|---|
| V_{L} 1 | QSVLTQPPSVSGAPGQRVTISC [symbol] WYQQVPGTAFKLLIY [symbol] GVPDRFSGSKSGTSASLAITGLQAEDEADYYC [symbol] GGGTRLTVL SEQ ID NO: 1 |
| V_{L} 2 | QSVLTQPPSVSGAPGQRVTISC [symbol] WYQQLPGTAPKLLIY [symbol] GVPDRFSGSKSGTSASLAITGLQAEDEADYYC [symbol] GGGTKLTVL SEQ ID NO: 3 |
| V_{L} 3 | QAVLTQPSSLSASPGASASLTC [symbol] WYQQKPGSFFQYLLR [symbol] GVPSRFSGSKDASANAGILLISGLQSEDEADYYC [symbol] FGGGTKLTVL SEQ ID NO: 4 |
| V_{L} 4 | QAVLTQPSSLSASPGASASLTC [symbol] WYQQKPGSFFQYLLR [symbol] GVPSRFSGSKDASANAGILLISGLQSEDEADYYC [symbol] FGGGTKLTVL SEQ ID NO: 4 |
| V_{L} 5 | QPVLTQPPSASASLGASVTLTC [symbol] WYQQRPGKGPRFVMR [symbol] GIPDRFSVLGSGLNRYLTIKNIQEEDESDYHC [symbol] FGTGTKVTVL SEQ ID NO: [symbol] |
| V_{L} 6 | QPVLTQPPSASASLGASVTLTC [symbol] WYQQRPGKGPRFVMR [symbol] GIPDRFSVLGSGLNRYLTIKNIQEEDESDYHC [symbol] FGTGTKVTVL SEQ ID NO: 9 |
| V_{L} 7 | QPELTQPPSASASLGASVTLTC [symbol] WYQLRPGKGPRFVMR [symbol] GIPDRFSVLGSGLNRSLTIKNIQEEDESDYHC [symbol] FGTGTKVTVL SEQ ID NO: 11 |
| V_{L} 8 | DIQLTPSPSSVSASVGDRVTITC [symbol] WYQQKPGKAPKLLIY [symbol] GVPSRFSGSGSGTDFTLTISSLQPEDFATYYC [symbol] FGGGTKVEIK SEQ ID NO: 13 |
| V_{L} 9 | DIQMTQSPSSVSASVGDRVTITC [symbol] WYQQKPGKAPSLLIY [symbol] GVPSRFSGSVSGTDFTLTISSLQPEDFATYYC [symbol] GPGTKVDFK SEQ ID NO: 15 |
| V_{L} 10 | DIQMTQSPSSVSASVGDRVTITC [symbol] WYQQKPGKAPKLLIY [symbol] GVPSRFSGSGSGTDFTLTISSLQPEDFATYYC [symbol] GPGTKVDIK SEQ ID NO: 17 |
| V_{L} 11 | DSQMTQSPSSVSASVGDRVTITC [symbol] WYQQKPGQAPNLLIY [symbol] GVPSRFSGSGSGTEFTLTISSLQPEDFATYYC [symbol] GPGTKVDIK SEQ ID NO: 19 |
| V_{L} 12 | DIQMTQSPSSVSASVGDRVTITC [symbol] WYQQKPGKAPKLLIY [symbol] GVPSRFSGSGSGTDFTLTISSLQPDQFATYYC [symbol] FGGGTKVEIK SEQ ID NO: 21 |
| V_{L} 13 | DIQMTQSPSSVSASVGDRVTITC [symbol] WYQQKPGKAPKLLIY [symbol] GVPSRFSGSGSGTDFTLTISSLQPEDFATYYC [symbol] GPGTKVDIK SEQ ID NO: 23 |
| V_{L} 14 | DIQLTQSPSSVSASVGDRVTITC [symbol] WYQQKPGKAPNLLIY [symbol] GVPSRFSGSGSGTDFTLTISSLQPADFATYFC [symbol] GPGTKVDVK SEQ ID NO: 25 |
| V_{L} 15 | DIQMTQSPSSVSASVGDRVTITC [symbol] WYQQKPGKAPKLLIY [symbol] GVPSRFSGSGSGTDFTLTISSLQPEDFATYYC [symbol] FGPGTKVDIK SEQ ID NO: 27 |
| V_{L} 15 | DIQMTQSPSSLSASVGDRVTITC [symbol] WYQQKPGKAPKRLIY [symbol] GVPSRFSGSGSGTEFTLTISSLQPEDFATYYC [symbol] FGQGTKVEIE SEQ ID NO: 29 |

[symbol]

indicates data missing or illegible when filed

TABLE 2
Exemplary Variant Heavy Chain Region Sequences

| | FR1 CDRH1 FR2 CDRH2 FR3 DRH3 FR4 |
|---|---|
| V_{H} 1 | QVQLVESGGGVVQPGRSLRLSCAASGFTFS [symbol] WVRQAPGKGLEWVA [symbol] RFTISRDNSKNTLYLQMNSLRAEDTAVYYCAR [symbol] WGQGTMVTVSS SEQ ID NO: 31 |
| V_{H} 2 | QVQLVESGGGVVQPGRSLRLSCAASGFTFS [symbol] WVRQAPGKGLEWVA [symbol] RFTISRDNSKNTLYQMNSLRAEDTAVYYCAR [symbol] WGQGTMVTVSS SEQ ID NO: 33 |
| V_{H} 3 | QVQLVESGGGVVQPGRSLRLSCAASGFTFS [symbol] WVRQAPGKGLEWVA [symbol] RFTISRDNSKNTLYLQMNSLRAEDTAVYYCAR [symbol] WGQGTLVTVSS SEQ ID NO: 34 |
| V_{H} 4 | QVQLVESGGGVVQPGRSLRLSCAASGFTFS [symbol] WVRQAPGKGLEWLS [symbol] RFTISRDNSKNTLYLQMNSLRAEDTAVYYCAR [symbol] WGQGTLVTVSS SEQ ID NO: 36 |
| V_{H} 5 | EVQLVESGGGLVQPGGSLRLSCAASGFTFS [symbol] WVRQAPGKGLEWVS [symbol] RFTISRDNAKNSLYLQMNSLRDEDTAVYYCAR [symbol] WGQGTTVTVSS SEQ ID NO: 38 |
| V_{H} 6 | EVQLVESGGGLVQPGGSLRLSCAASGFTFS [symbol] WVRQAPGKGLEWVS [symbol] RFTISRDNAKNSLYLQMNSLRDEDTAVYYCAR [symbol] WGQGTTVTVSS SEQ ID NO: 40 |
| V_{H} 7 | EVQLVESGGGLVQPGGSLRLSCVVSGFTFS [symbol] WVRQAPGKGLEWVS [symbol] RFTISRDNAKNSLYLQMNSLRDEDTAVYYCAR [symbol] WGQGTTVTVSS SEQ ID NO: 42 |
| V_{H} 8 | QVQLQESGPGLVKPSETLSLTCTVSGGSIS [symbol] WIRQPAGKGLEWIG [symbol] RVTMSLDTSKNQFSLRLTSVTAADTAVYYCAR [symbol] WGQGTTVTVSS SEQ ID NO: 44 |
| V_{H} 9 | QVQLQESGPGLVKPSQTLSLTCTVSGGSISSSGG [symbol] WIRQHPGKGLEWIG [symbol] VTISVDTSKNQFSLKLSSVTAADTAVYYCAK [symbol] VWGQGTTVTVSS SEQ ID NO: 46 |
| V_{H} 10 | QVQLQESGPGLVKPSQTLSLTCTVSGGS [symbol] WIRQHPGKGLEWIG [symbol] RVTISVDTSQNQFSLKSSVTAADTAVYYCAR [symbol] WGQGTTVTVSS SEQ ID NO: 48 |
| V_{H} 11 | QVQLQESGPGLVKPSQTLSLTCTVSGGSIS [symbol] WIRQHPGKGLEWIG [symbol] RVTISVDTSKNQFSLKLSSVTAADTAVYYCAR [symbol] WGQGTTVTVSS SEQ ID NO: 50 |
| V_{H} 12 | QVQLQESGPRLVKPSETLSLTCTVSGDSIS [symbol] WIRQPPGKGLEWLG [symbol] RVTISIDTSKNQFSLKLSSVTAADTAVYYCTR [symbol] WGQGTLVTVSS SEQ ID NO: 52 |
| V_{H} 13 | QVQLQESGPGLVKPSQTLSLTCTVSGGSIS [symbol] WIRQHPGKGLEWIG [symbol] RITISVDTSKNQFSLSLSSVTAADTAVYYCAR [symbol] WGQGTTVTVSS SEQ ID NO: 54 |
| V_{H} 14 | QVQLQESGPGLVKPSQTLSLTCTVSGGSISSG [symbol] WIRQHPGKGLEWIG [symbol] RVTMSVDTSKNQFSLKLSSVTAADTAVYYCAK [symbol] WGQGTTVTVSS SEQ ID NO: 56 |
| V_{H} 15 | QVQLQESGPGLVKPSQTLSLTCTVSGGSINSG [symbol] WIRQHPGKGLEWIG [symbol] RVTISVDTSKNQFSLKLSSVTAADTAVYYCAR [symbol] WGQGTTVTVSS SEQ ID NO: 58 |
| V_{H} 16 | QVQLVESGGGVVQPGRSLRLSCAASGFTFS [symbol] WVRQAPGKGLEWVA [symbol] RFTISRDNSKNTLYLQMNSLRAEDTAVYYCAR [symbol] WGQGTTVTVSS SEQ ID NO: 60 |

For these calculations, gaps in alignments (if any) must be addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073.

In calculating percent identity, the sequences being compared are aligned in a way that gives the largest match between the sequences. The computer program used to determine percent identity is the GCG program package, which includes GAP (Devereux et al., 1984, Nucl. Acids Res. 12:387; Genetics Computer Group, University of Wisconsin, Madison, Wis.). The computer algorithm GAP is used to align the two polypeptides or polynucleotides for which the percent sequence identity is to be determined. The sequences are aligned for optimal matching of their respective amino acid or nucleotide (the “matched span”, as determined by the algorithm). A gap opening penalty (which is calculated as 3× the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm.

Recommended parameters for determining percent identity for polypeptides or nucleotide sequences using the GAP program are the following: Algorithm: Needleman et al., 1970, J. Mol. Biol. 48:443-453; Comparison matrix: BLOSUM 62 from Henikoff et al., 1992, supra; Gap Penalty: 12 (but with no penalty for end gaps), Gap Length Penalty: 4, Threshold of Similarity: 0. Certain alignment schemes for aligning two amino acid sequences may result in matching of only a short region of the two sequences and this small aligned region may have very high sequence identity even though there is no significant relationship between the two full-length sequences. Accordingly, the selected alignment method (GAP program) can be adjusted if so desired to result in an alignment that spans at least 50 contiguous amino acids of the target polypeptide. The heavy and light chain variable regions disclosed herein include consensus sequences derived from groups of related antigen-binding proteins. The amino acid sequences of the heavy and light chain variable regions were analyzed for similarities. Four groups emerged, one group having kappa light chain variable regions, (VH9/VL9, VH10/VL10, VH11/VL11, VH13/VL13, VH14/VL14 and VH15/VL15) and three groups having lambda light chain variable regions: lambda group 1 (VH5/VAS, VH6/VL6 and VH7/VL7), lambda group 2 (VH3/VL3 and VH4/VL4), and lambda group 3 (VH1/VL1 and VH2/VL2). Light chain germlines represented include VK1/A30 and VK1/L19. Light chain lambda germlines represented include VL1/1e, VL3/3p, VL5/5c and VL9/9a. Heavy chain germlines represented include VH3/3-30, VH3/3-30.3, VH3/3-33, VH3/3-48, VH4/4-31 and VH4/4-59. As used herein, a “consensus sequence” refers to amino acid sequences having conserved amino acids common among a number of sequences and variable amino acids that vary within given amino acid sequences. Consensus sequences may be determined using standard phylogenic analyses of the light and heavy chain variable regions corresponding to the IL-23 antigen-binding proteins disclosed herein.

The light chain variable region consensus sequence for the kappa group is DX₁QX₂TQSPSSVSASVG DRVTITCRASQGX₃X₄SX₅WX₆AWYQQKPGX₇APX₈LL IYAASSLQSGVPSRFSGSXOSGTX₁₀FTLTISSLQPX₁₁DFATYX₁₂CQQANSFPFTFGPGTKVDX₁₃K (SEQ ID NO:30), where X₁ is selected from I or S; X₂ is selected from M or L; X₃ is selected from G or V and X₄ is selected from S, F or I; X₅ is selected from S or G; X₆ is selected from F or L; X₇ is selected from K or Q; X₈ is selected from K, N or S; X₉ is selected from G or V; X₁₀ is selected from D or E, X₁₁ is selected from E or A; X₁₂ is selected from Y or F; and X₁₃ is selected from I, V or F.

The light chain variable region consensus sequence for lambda group 1 is QPX₁LTQPPSASASLGASVTLTCTLX₂SGYSDYKVDWYQX₃RPGKGPRFVMRVGTGGX₄VGSK GX₅GIPDRFSVLGSGLNRX₆LTIKNIQEEDESDYHCGADHGSGX₇NFVYVFGTGTKVTVL (SEQ ID NO:61), where X₁ is selected from V or E; X₂ is selected from N or S; X₃ is selected from Q or L and X₄ is selected from I or T; X₅ is selected from D or E; X₆ is selected from Y or S; and X₇ is selected from S or N.

The light chain variable region consensus sequence for lambda group 3 is QSVLTQ PPSVSGAPGQRVTISCTGSSSNX₁GAGYDVHWYQQX₂PGTAPKLLIYGSX₃NRPSGVPDRFSG SKSGTSASLAITGLQAEDEADYYCQSYDSSLSGWVFGGGTX₄RLTVL (SEQ ID NO:139), where X₁ is selected from T or I; X₂ is selected from V or L; X₃ is selected from G or N and X₄ is selected from R or K.

The heavy chain variable region consensus sequence for the kappa group is QVQLQESGPGLVKPSQTLSLTCTVSGGSIX₁SGGYYWX₂WIRQHPGKGLEWIGX₃X₄YSGX₅X₆Y YNPSLKSRX₇TX₈SVDTSX₉NQFSLX₁₀LSSVTAADTAVYYCAX₁₁X₁₂RGX₁₃YYGMDVWGQGTTV TVSS (SEQ ID NO:140), where X₁ is selected from N or S; X₂ is selected from S or T; X₃ is selected from Y or H; X₄ is selected from Y or H; X₅ is selected from S or N; X₆ is selected from S or T; X₇ is selected from V or I; X₈ is selected from I or M; X₉ is selected from K or Q; X₁₀ is selected from K or S, X₁₁ is selected from R or K; X₁₂ is selected from D or N; and X₁₃ is selected from H, F or Y.

The heavy chain variable region consensus sequence for lambda group 1 is EVQLVESGGGLVQPGGSLRLSCX₁X₂SGFTFSX₃X₄SMNWVRQAPGKGLEWVSYISSX₅SSTX₆Y X₇ADSVKGRFTISRDNAKNSLYLQMNSLRDEDTAVYYCARRIAAAGX₈X₉X₁₀YYYAX₁₁DVWGQG TTVTVSS (SEQ ID NO:141), where X₁ is selected from A or V; X₂ is selected from A or V; X₃ is selected from T or S; X₄ is selected from Y or F; X₅ is selected from S or R; X₆ is selected from R or I; X₇ is selected from H, Y or I; X₈ is selected from P or G; X₉ is selected from W or F; X₁₀ is selected from G or H and X₁₁ is selected from M or L.

The heavy chain variable region consensus sequence for lambda group 2 is QVQLVESGGGVVQPG RSLRLSCAASGFTFSSYX₁M HWVRQAPGKGLEWX₂X₃VISX₄DGSX₅KYYADSVKGRFTISRDNSKNTLYLQMNSLRAEDTAVY YCARERTTLSGSYFDYWGQGTLVTVSS (SEQ ID NO:142), where X₁ is selected from G or A; X₂ is selected from V or L; X₃ is selected from A or S; X₄ is selected from F or H; and X₅ is selected from L or I.

The heavy chain variable region consensus sequence for lambda group 3 is QVQLVESGGGVVQPGRSLRLSCAASGFTFSSYGMHWVRQAPGKGLEWVAVIWYDGSNX₁YY ADSVKGRFTISRDNSKNTLYLQMNSLRAEDTAVYYCARDRGYX₂SSWYPDAFDIWGQGTMVT VSS (SEQ ID NO:143), where X₁ is selected from E or K and X₂ is selected from T or S.

### Complementarity Determining Regions

Complementarity determining regions or “CDRs” are embedded within a framework in the heavy and light chain variable regions where they constitute the regions responsible for antigen binding and recognition. Variable domains of immunoglobulin chains of the same species, for example, generally exhibit a similar overall structure; comprising relatively conserved framework regions (FR) joined by hypervariable CDR regions. An antigen-binding protein can have 1, 2, 3, 4, 5, 6 or more CDRs. The variable regions discussed above, for example, typically comprise three CDRs. The CDRs from heavy chain variable regions and light chain variable regions are typically aligned by the framework regions to form a structure that binds specifically on a target antigen (e.g., IL-23). From N-terminal to C-terminal, naturally-occurring light and heavy chain variable regions both typically conform to the following order of these elements: FR1, CDR1, FR2, CDR2, FR3, CDR3 and FR4. The CDR and FR regions of exemplary light chain variable domains and heavy chain variable domains are highlighted in Tables 1 and 2. It is recognized that the boundaries of the CDR and FR regions can vary from those highlighted. Numbering systems have been devised for assigning numbers to amino acids that occupy positions in each of these domains. Complementarity determining regions and framework regions of a given antigen-binding protein may be identified using these systems. Numbering systems are defined in Kabat et al., Sequences of Proteins of Immunological Interest, 5th Ed., US Dept. of Health and Human Services, PHS, NIH, NIH Publication No. 91-3242, 1991, or Chothia & Lesk, 1987, J. Mol. Biol. 196:901-917; Chothia et al., 1989, Nature 342:878-883. Other numbering systems for the amino acids in immunoglobulin chains include IMGT® (the international ImMunoGeneTics information system; Lefranc et al, Dev. Comp. Immunol. 2005, 29:185-203); and AHo (Honegger and Pluckthun, J. Mol. Biol. 2001, 309(3):657-670). The CDRs provided herein may not only be used to define the antigen-binding domain of a traditional antibody structure, but may be embedded in a variety of other polypeptide structures, as described herein.

The antigen-binding proteins disclosed herein are polypeptides into which one or more CDRs may be grafted, inserted, embedded and/or joined. An antigen-binding protein can have, for example, one heavy chain CDR1 (“CDRH1”), and/or one heavy chain CDR2 (“CDRH2”), and/or one heavy chain CDR3 (“CDRH3”), and/or one light chain CDR1 (“CDRL1”), and/or one light chain CDR2 (“CDRL2”), and/or one light chain CDR3 (“CDRL3”). Some antigen-binding proteins include both a CDRH3 and a CDRL3. Specific embodiments generally utilize combinations of CDRs that are non-repetitive, e.g., antigen-binding proteins are generally not made with two CDRH2 regions in one variable heavy chain region, and the like. Antigen-binding proteins may comprise one or more amino acid sequences that are identical to or that differ from to the amino acid sequences of one or more of the CDRs presented in Table 3 at only 1, 2, 3, 4, 5, 6, 7, 8, 9, 10, 11, 12, 13, 14 or 15 amino acid residues, wherein each such sequence difference is independently either a deletion, insertion or substitution of one amino acid. The CDRs in some antigen-binding proteins comprise sequences of amino acids that have at least 80%, 85%, 90%, 91%, 92, 93%, 94%, 95%, 96%, 97%, 98%, or 99% sequence identity to CDRs sequence listed in Table 3. In some antigen-binding proteins, the CDRs are embedded into a “framework” region, which orients the CDR(s) such that the proper antigen binding properties of the CDR(s) is achieved.

TABLE 3
Exemplary CDRH and CDRL Sequences
Exemplary CDRL Sequences

| CDRL1 | CDRL2 | CDRL3 |
|---|---|---|
| TGSSSNTGAGYDVH | GSGNRPS | QSYDSSLSGWV |
| SEQ ID NO: 62 | SEQ ID NO: 63 | SEQ ID NO: 64 |
| TGSSSNIGAGYDVH | GSNNRPS | MIWHSSASV |
| SEQ ID NO: 65 | SEQ ID NO: 66 | SEQ ID NO: 67 |
| TLRSGINVGTYRIY | YKSDSDKQQGS | GADHGSGSNFVYV |
| SEQ ID NO: 68 | SEQ ID NO: 69 | SEQ ID NO: 70 |
| TLNSGYSDYKV | VGTGGIVGSKGD | GADHGSGNNFVYV |
| SEQ ID NO: 71 | SEQ ID NO: 72 | SEQ ID NO: 73 |
| TLSSGYSDYKV | VGTGGIVGSKGE | QQANSFPFT |
| SEQ ID NO: 74 | SEQ ID NO: 75 | SEQ ID NO: 76 |
| RASQGFSGWLA | VGTGGTVGSKGE | QQATSFPLT |
| SEQ ID NO: 77 | SEQ ID NO: 78 | SEQ ID NO: 79 |
| RASQVISSWLA | AASSLQS | QQADSFPPT |
| SEQ ID NO: 80 | SEQ ID NO: 81 | SEQ ID NO: 82 |
| RASQVISSWFA | | LQHNSYPPT |
| SEQ ID NO: 83 | | SEQ ID NO: 84 |
| RASQGSSSWFA | | |
| SEQ ID NO: 85 | | |
| RASQGISSWFA | | |
| SEQ ID NO: 86 | | |
| RAGQVISSWLA | | |
| SEQ ID NO: 87 | | |
| RASQGIAGWLA | | |
| SEQ ID NO: 88 | | |
| RASQGIRNDLG | | |
| SEQ ID NO: 89 | | |

Exemplary CDRH Sequences

| CDRH1 | CDRH2 | CDRH3 |
|---|---|---|
| SYGNAH | VIWYDGSNEYYADSVKG | DRGYTSSWYPDAFDI |
| SEQ ID NO: 91 | SEQ ID NO: 92 | SEQ ID NO: 93 |
| SYAMH | VIWYDGSNKYYADSVKG | DRGYSSSWYPDAFDI |
| SEQ ID NO: 94 | SEQ ID NO: 95 | SEQ ID NO: 96 |
| TYSMN | VISFDGSLKYYADSVKG | ERTTLSGSYFDY |
| SEQ ID NO: 97 | SEQ ID NO: 98 | SEQ ID NO: 99 |
| SYSMN | VISHDGSIKYYADSVKG | RIAAAGGFHYYYALDV |
| SEQ ID NO: 100 | SEQ ID NO: 101 | SEQ ID NO: 102 |
| SFSMN | YISSRSSTIYIADSVKG | RIAAAGPWGYYYMADV |
| SEQ ID NO: 103 | SEQ ID NO: 104 | SEQ ID NO: 105 |
| SGGYYWT | YISSSSSTRYHADSVKG | NRGYYYGMDV |
| SEQ ID NO: 106 | SEQ ID NO: 107 | SEQ ID NO: 108 |
| SGGYYWS | YISSRSSTIYYADSVKG | NRGFYYGMDV |
| SEQ ID NO: 109 | SEQ ID NO: 110 | SEQ ID NO: 111 |
| SYFWS | YIYYSGNTYYNPSLKS | DRGHYYGMDV |
| SEQ ID NO: 112 | SEQ ID NO: 113 | SEQ ID NO: 114 |
| TYYWS | HIHYSGNTYYNPSLKS | DRGSYYGSDY |
| SEQ ID NO: 115 | SEQ ID NO: 116 | SEQ ID NO: 117 |
| | YIYYSGSTYYNPSLKS | DRGYYYGVDV |
| | SEQ ID NO: 118 | SEQ ID NO: 119 |
| | YIYYSGSSYYNPSLKS | ENTVTIYYNYGMDV |
| | SEQ ID NO: 120 | SEQ ID NO: 6 |
| | YIYYSGSTNYNPSLKS | |
| | SEQ ID NO: 121 | |
| | LIYTSGSTNYNPSLKS | |
| | SEQ ID NO: 122 | |
| | LIWYDGSNKYYADSVKG | |
| | SEQ ID NO: 90 | |

Provided herein are CDR1 regions comprising amino acid residues 23-34 of SEQ ID NOs:7 and 11; amino acid residues 24-34 of SEQ ID NOs: 9, 13, 15, 17, 19, 21, 23, 25, 27 and 29; amino acid residues 23-36 of SEQ ID NOs: 1, 3 and 4; amino acid residues 31-35 of SEQ ID NOs:31, 33, 34, 38, 40, 44, 52 and 60 and amino acid residues 31-37 or SEQ ID NOs: 46, 48, 50, 54, 56 and 58.

CDR2 regions are provided comprising amino acid residues 50-56 of SEQ ID NOs: 9, 13, 15, 17, 19, 21, 23, 25, 27 and 29; amino acid residues 50-61 of SEQ ID NOs: 7 and 11; amino acid residues 52-62 of SEQ ID NO:4; amino acid residues 50-65 of SEQ ID NOs: 31, 33, 44 and 52; amino acid residues 50-66 of SEQ ID NOs: 36, 38, 40, 42 and 60; amino acid residues 52-58 of SEQ ID NOs: 1 and 3 and amino acid residues 52-67 of SEQ ID NOs: 46, 48, 50, 54, 56 and 58.

CDR3 regions comprising amino acid residues 89-97 of SEQ ID NOs: 13, 15, 17, 19, 21, 23, 25, 27 and 29; amino acid residues 91-101 of SEQ ID NOs: 1 and 3; amino acid residues 94-106 of SEQ ID NOs: 7, 9 and 11; amino acid residues 98-107 of SEQ ID NOs: 44 and 52; amino acid residues 97-105 of SEQ ID NO: 4; amino acid residues 99-110 of SEQ ID NOs: 34 and 36; amino acid residues 99-112 of SEQ ID NO: 112; amino acid residues 99-113 of SEQ ID NOs: 31 and 33; amino acid residues 99-114 of SEQ ID NOs: 38, 40 and 42; amino acid residues 100-109 of SEQ ID NOs: 46, 48, 54, 56 and 58; and amino acid residues 101-019 of SEQ ID NO; 50; are also provided.

The CDRs disclosed herein include consensus sequences derived from groups of related sequences. As described previously, four groups of variable region sequences were identified, a kappa group and three lambda groups. The CDRL1 consensus sequence from the kappa group consists of RASQX₁X₂SX₃WX₄A (SEQ ID NO:123), where X₁ is selected from G or V; X₂ is selected from I, F or S; X₃ is selected from S or G and X₄ is selected from F or L. The CDRL1 consensus sequence from lambda group 1 consists of TLX₁SGYSDYKVD (SEQ ID NO:124), wherein X₁ is selected from N or S. The CDRL1 consensus sequences from lambda group 3 consists of TGSSSNX₁GAGYDVH (SEQ ID NO:125), wherein X₁ is selected from I or T.

The CDRL2 consensus sequence from lambda group 1 consists of VGTGGX₁VGSKGX₂ (SEQ ID NO: 126), wherein X₁ is selected from I or T and X₂ is selected from D or E. The CDRL2 consensus sequence from lambda group 3 consists of GSX₁NRPS (SEQ ID NO:127), wherein X₁ is selected from N or G.

The CDRL3 consensus sequences include GADHGSGX¹NFVYV (SEQ ID NO:128), wherein X₁ is S or N.

The CDRH1 consensus sequence from the kappa group consists of SGGYYWX₁ (SEQ ID NO:129), wherein X₁ is selected from S or T. The CDRH₁ consensus sequence from lambda group 1 consists of X₁X₂SMN (SEQ ID NO:131), wherein X₁ is selected from S or T and X₂ is selected from Y or F. The CDRH1 consensus sequence from lambda group 2 consists of SYX₁MH (SEQ ID NO:130), wherein X₁ is selected from G or A.

The CDRH2 consensus sequence from the kappa group consists of X₁IX₂YSGX₃X₄YYNPSLKS (SEQ ID NO:132), wherein X₁ is selected from Y or H; X₂ is selected from Y or H; X₃ is selected from S or N; and X₄ is selected from T or S. The consensus sequence from lambda group 1 consists of YISSX₁SSTX₂YX₃ADSVKG (SEQ ID NO:134), wherein X₁ is selected from R or S, X₂ is selected from I or R, and X₃ is selected from I, H or Y. The consensus sequence from lambda group 2 consists of VISX₁DGSX₂KYYADSVKG (SEQ ID NO:133), wherein X₁ is F or H and X₂ is L or T. The CDRH2 consensus sequence from lambda group 3 consists of VIWYDGSNX₁YYADSVKG (SEQ ID NO:135), wherein X₁ is selected from K or E.

The CDRH3 consensus sequence from the kappa group consists of X₁RGX₂YYGMDV (SEQ ID NO:136), wherein X₁ is selected from N or D and X₂ is selected from H, Y or F. The CDRH3 consensus sequence from lambda group 1 consists of RIAAAGX₁X₂X₃YYYAX₄DV (SEQ ID NO:137), wherein X₁ is selected from G or P; X₂ is selected from F or W; X₃ is selected from H or G and X₄ is selected from L and M. The CDRH3 consensus sequence from lambda group 3 consists of DRGYX₁SSWYPDAFDI (SEQ ID NO:138), wherein X₁ is selected from S or T.

### Monoclonal Antibodies

The antigen-binding proteins that are provided include monoclonal antibodies that bind to IL-23. Monoclonal antibodies may be produced using any technique known in the art, e.g., by immortalizing spleen cells harvested from the transgenic animal after completion of the immunization schedule. The spleen cells can be immortalized using any technique known in the art, e.g., by fusing them with myeloma cells to produce hybridomas. Myeloma cells for use in hybridoma-producing fusion procedures preferably are non-antibody-producing, have high fusion efficiency, and enzyme deficiencies that render them incapable of growing in certain selective media which support the growth of only the desired fused cells (hybridomas). Examples of suitable cell lines for use in mouse fusions include Sp-20, P3-X63/Ag8, P3-X63-Ag8.653, NS1/1.Ag 4 1, Sp210-Ag14, FO, NSO/U, MPC-11, MPC11-X45-GTG 1.7 and S194/5XXO Bul; examples of cell lines used in rat fusions include R210.RCY3, Y3-Ag 1.2.3, IR983F and 413210. Other cell lines useful for cell fusions are U-266, GM1500-GRG2, LICR-LON-HMy2 and UC729-6.

In some instances, a hybridoma cell line is produced by immunizing an animal (e.g., a transgenic animal having human immunoglobulin sequences) with an IL-23 immunogen; harvesting spleen cells from the immunized animal; fusing the harvested spleen cells to a myeloma cell line, thereby generating hybridoma cells; establishing hybridoma cell lines from the hybridoma cells, and identifying a hybridoma cell line that produces an antibody that binds an IL-23 polypeptide while sparing IL-12.

Monoclonal antibodies secreted by a hybridoma cell line can be purified using any technique known in the art. Hybridomas or monoclonal antibodies (mAbs) may be further screened to identify mAbs with particular properties, such as the ability to inhibit IL-23-induced activity.

### Chimeric and Humanized Antibodies

Chimeric and humanized antibodies based upon the foregoing sequences are also provided. Monoclonal antibodies for use as therapeutic agents may be modified in various ways prior to use. One example is a chimeric antibody, which is an antibody composed of protein segments from different antibodies that are covalently joined to produce functional immunoglobulin light or heavy chains or immunologically functional portions thereof. Generally, a portion of the heavy chain and/or light chain is identical with or homologous to a corresponding sequence in antibodies derived from a particular species or belonging to a particular antibody class or subclass, while the remainder of the chain(s) is/are identical with or homologous to a corresponding sequence in antibodies derived from another species or belonging to another antibody class or subclass. For methods relating to chimeric antibodies, see, for example, U.S. Pat. No. 4,816,567; and Morrison et al., 1985, Proc. Natl. Acad. Sci. USA 81:6851-6855. CDR grafting is described, for example, in U.S. Pat. Nos. 6,180,370, 5,693,762, 5,693,761, 5,585,089, and 5,530,101.

One useful type of chimeric antibody is a “humanized” antibody. Generally, a humanized antibody is produced from a monoclonal antibody raised initially in a non-human animal. Certain amino acid residues in this monoclonal antibody, typically from non-antigen recognizing portions of the antibody, are modified to be homologous to corresponding residues in a human antibody of corresponding isotype. Humanization can be performed, for example, using various methods by substituting at least a portion of a rodent variable region for the corresponding regions of a human antibody (see, e.g., U.S. Pat. Nos. 5,585,089, and 5,693,762; Jones et al., 1986, Nature 321:522-525; Riechmann et al., 1988, Nature 332:323-27; Verhoeyen et al., 1988, Science 239:1534-1536).

In certain embodiments, constant regions from species other than human can be used along with the human variable region(s) to produce hybrid antibodies.

### Fully Human Antibodies

Fully human antibodies are also provided. Methods are available for making fully human antibodies specific for a given antigen without exposing human beings to the antigen (“fully human antibodies”). One specific means provided for implementing the production of fully human antibodies is the “humanization” of the mouse humoral immune system. Introduction of human immunoglobulin (Ig) loci into mice in which the endogenous Ig genes have been inactivated is one means of producing fully human monoclonal antibodies (mAbs) in mouse, an animal that can be immunized with any desirable antigen. Using fully human antibodies can minimize the immunogenic and allergic responses that can sometimes be caused by administering mouse or mouse-derivatized mAbs to humans as therapeutic agents.

Fully human antibodies can be produced by immunizing transgenic animals (usually mice) that are capable of producing a repertoire of human antibodies in the absence of endogenous immunoglobulin production. Antigens for this purpose typically have six or more contiguous amino acids, and optionally are conjugated to a carrier, such as a hapten. See, e.g., Jakobovits et al., 1993, Proc. Natl. Acad. Sci. USA 90:2551-2555; Jakobovits et al., 1993, Nature 362:255-258; and Bruggermann et al., 1993, Year in Immunol. 7:33. In one example of such a method, transgenic animals are produced by incapacitating the endogenous mouse immunoglobulin loci encoding the mouse heavy and light immunoglobulin chains therein, and inserting into the mouse genome large fragments of human genome DNA containing loci that encode human heavy and light chain proteins. Partially modified animals, which have less than the full complement of human immunoglobulin loci, are then cross-bred to obtain an animal having all of the desired immune system modifications. When administered an immunogen, these transgenic animals produce antibodies that are immunospecific for the immunogen but have human rather than murine amino acid sequences, including the variable regions. For further details of such methods, see, for example, WIPO patent publications WO96/33735 and WO94/02602. Additional methods relating to transgenic mice for making human

antibodies are described in U.S. Pat. Nos. 5,545,807; 6,713,610; 6,673,986; 6,162,963; 5,545,807; 6,300,129; 6,255,458; 5,877,397; 5,874,299 and 5,545,806; in WIPO patent publications WO91/10741, WO90/04036, and in EP 546073B1 and EP 546073A1.

The transgenic mice described above contain a human immunoglobulin gene minilocus that encodes unrearranged human heavy ([mu] and [gamma]) and [kappa] light chain immunoglobulin sequences, together with targeted mutations that inactivate the endogenous [mu] and [kappa] chain loci (Lonberg et al., 1994, Nature 368:856-859). Accordingly, the mice exhibit reduced expression of mouse IgM or [kappa] and in response to immunization, and the introduced human heavy and light chain transgenes undergo class switching and somatic mutation to generate high affinity human IgG [kappa] monoclonal antibodies (Lonberg et al., supra.; Lonberg and Huszar, 1995, Intern. Rev. Immunol. 13: 65-93; Harding and Lonberg, 1995, Ann. N.Y. Acad. Sci. 764:536-546). The preparation of such mice is described in detail in Taylor et al., 1992, Nucleic Acids Research 20:6287-6295; Chen et al., 1993, International Immunology 5:647-656; Tuaillon et al., 1994, J. Immunol. 152:2912-2920; Lonberg et al., 1994, Nature 368:856-859; Lonberg, 1994, Handbook of Exp. Pharmacology 113:49-101; Taylor et al., 1994, International Immunology 6:579-591; Lonberg and Huszar, 1995, Intern. Rev. Immunol. 13:65-93; Harding and Lonberg, 1995, Ann. N.Y. Acad. Sci. 764:536-546; Fishwild et al., 1996, Nature Biotechnology 14:845-85. See, further U.S. Pat. Nos. 5,545,806; 5,569,825; 5,625,126; 5,633,425; 5,789,650; 5,877,397; 5,661,016; 5,814,318; 5,874,299; and 5,770,429; as well as U.S. Pat. No. 5,545,807; WIPO Publication Nos. WO 93/1227; WO 92/22646; and WO 92/03918. Technologies utilized for producing human antibodies in these transgenic mice are disclosed also in WIPO Publication No. WO 98/24893, and Mendez et al., 1997, Nature Genetics 15:146-156. For example, the HCo7 and HCo12 transgenic mice strains can be used to generate anti-IL-23 antibodies.

Using hybridoma technology, antigen-specific human mAbs with the desired specificity can be produced and selected from the transgenic mice such as those described above. Such antibodies may be cloned and expressed using a suitable vector and host cell, or the antibodies can be harvested from cultured hybridoma cells.

Fully human antibodies can also be derived from phage-display libraries (such as disclosed in Hoogenboom et al., 1991, J. Mol. Biol. 227:381; Marks et al., 1991, J. Mol. Biol. 222:581; WIPO Publication No. WO 99/10494). Phage display techniques mimic immune selection through the display of antibody repertoires on the surface of filamentous bacteriophage, and subsequent selection of phage by their binding to an antigen of choice.

### Bispecific or Bifunctional Antigen-Binding Proteins

A “bispecific,” “dual-specific” or “bifunctional” antigen-binding protein or antibody is a hybrid antigen-binding protein or antibody, respectively, having two different antigen-binding sites, such as one or more CDRs or one or more variable regions as described above. In some instances they are an artificial hybrid antibody having two different heavy/light chain pairs and two different binding sites. Multi-specific antigen-binding protein or “multispecific antibody” is one that targets more than one antigen or epitope. Bispecific antigen-binding proteins and antibodies are a species of multi-specific antigen-binding protein antibody and may be produced by a variety of methods including, but not limited to, fusion of hybridomas or linking of Fab′ fragments. See, e.g., Songsivilai and Lachmann, 1990, Clin. Exp. Immunol. 79:315-321; Kostelny et al., 1992, J. Immunol. 148:1547-1553.

### Immunological Fragments

Antigen-binding proteins also include immunological fragments of an antibody (e.g., a Fab, a Fab′, a F(ab′)2, or a scFv). A “Fab fragment” is comprised one light chain (the light chain variable region (VL) and its corresponding constant domain (CL)) and one heavy chain (the heavy chain variable region (VH) and first constant domain (CH1)). The heavy chain of a Fab molecule cannot form a disulfide bond with another heavy chain molecule. A “Fab′ fragment” contains one light chain and a portion of one heavy chain that also contains the region between the CH1 and CH2 domains, such that an interchain disulfide bond can be formed between the two heavy chains of two Fab′ fragments to form an F(ab′)2 molecule. A “F(ab′)2 fragment” thus is composed of two Fab′ fragments that are held together by a disulfide bond between the two heavy chains. A “Fv fragment” consists of the variable light chain region and variable heavy chain region of a single arm of an antibody. Single-chain antibodies “scFv” are Fv molecules in which the heavy and light chain variable regions have been connected by a flexible linker to form a single polypeptide chain, which forms an antigen-binding region. Single chain antibodies are discussed in detail in WIPO Publication No. WO 88/01649, U.S. Pat. Nos. 4,946,778 and 5,260,203; Bird, 1988, Science 242:423; Huston et al., 1988, Proc. Natl. Acad. Sci. U.S.A. 85:5879; Ward et al., 1989, Nature 334:544, de Graaf et al., 2002, Methods Mol Biol. 178:379-387; Kortt et al., 1997, Prot. Eng. 10:423; Kortt et al., 2001, Biomol. Eng. 18:95-108 and Kriangkum et al., 2001, Biomol. Eng. 18:31-40. A “Fc” region contains two heavy chain fragments comprising the CH1 and CH2 domains of an antibody. The two heavy chain fragments are held together by two or more disulfide bonds and by hydrophobic interactions of the CH3 domains.

Also included are domain antibodies, immunologically functional immunoglobulin fragments containing only the variable region of a heavy chain or the variable region of a light chain. In some instances, two or more VH regions are covalently joined with a peptide linker to create a bivalent domain antibody. The two VH regions of a bivalent domain antibody may target the same or different antigens. Diabodies are bivalent antibodies comprising two polypeptide chains, wherein each polypeptide chain comprises VH and VL domains joined by a linker that is too short to allow for pairing between two domains on the same chain, thus allowing each domain to pair with a complementary domain on another polypeptide chain (see, e.g., Holliger et al., Proc. Natl. Acad. Sci. USA 90:6444-48, 1993 and Poljak et al., Structure 2:1121-23, 1994). Similarly, tribodies and tetrabodies are antibodies comprising three and four polypeptide chains, respectively, and forming three and four antigen-binding sites, respectively, which can be the same or different. Maxibodies comprise bivalent scFvs covalently attached to the Fc region of IgGi, (see, e.g., Fredericks et al, 2004, Protein Engineering, Design & Selection, 17:95-106; Powers et al., 2001, Journal of Immunological Methods, 251:123-135; Shu et al., 1993, Proc. Natl. Acad. Sci. USA 90:7995-7999; Hayden et al., 1994, Therapeutic Immunology 1:3-15).

### Various Other Forms

Also provided are variant forms of the antigen-binding proteins disclosed above, some of the antigen-binding proteins having, for example, one or more conservative amino acid substitutions in one or more of the heavy or light chains, variable regions or CDRs listed in Tables 1 and 2. Naturally-occurring amino acids may be divided into classes based on common side chain properties: hydrophobic (norleucine, Met, Ala, Val, Leu, lie); neutral hydrophilic (Cys, Ser, Thr, Asn, Gln); acidic (Asp, Glu); basic (His, Lys, Arg); residues that influence chain orientation (Gly, Pro); and aromatic (Trp, Tyr, Phe).

Conservative amino acid substitutions may involve exchange of a member of one of these classes with another member of the same class. Conservative amino acid substitutions may encompass non-naturally occurring amino acid residues, which are typically incorporated by chemical peptide synthesis rather than by synthesis in biological systems. These include peptidomimetics and other reversed or inverted forms of amino acid moieties. Such substantial modifications in the functional and/or biochemical characteristics of the antigen-binding proteins described herein may be achieved by creating substitutions in the amino acid sequence of the heavy and light chains that differ significantly in their effect on maintaining (a) the structure of the molecular backbone in the area of the substitution, for example, as a sheet or helical conformation, (b) the charge or hydrophobicity of the molecule at the target site, or (c) the bulkiness of the side chain.

Non-conservative substitutions may involve the exchange of a member of one of the above classes for a member from another class. Such substituted residues may be introduced into regions of the antibody that are homologous with human antibodies, or into the non-homologous regions of the molecule.

In making such changes, according to certain embodiments, the hydropathic index of amino acids may be considered. The hydropathic profile of a protein is calculated by assigning each amino acid a numerical value (“hydropathy index”) and then repetitively averaging these values along the peptide chain. Each amino acid has been assigned a hydropathic index on the basis of its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic profile in conferring interactive biological function on a protein is understood in the art (see, e.g., Kyte et al., 1982, J. Mol. Biol. 157:105-131). It is known that certain amino acids may be substituted for other amino acids having a similar hydropathic index or score and still retain a similar biological activity. In making changes based upon the hydropathic index, in certain embodiments, the substitution of amino acids whose hydropathic indices are within ±2 is included. In some aspects, those which are within ±1 are included, and in other aspects, those within ±0.5 are included.

It is also understood in the art that the substitution of like amino acids can be made effectively on the basis of hydrophilicity, particularly where the biologically functional protein or peptide thereby created is intended for use in immunological embodiments, as in the present case. In certain embodiments, the greatest local average hydrophilicity of a protein, as governed by the hydrophilicity of its adjacent amino acids, correlates with its immunogenicity and antigen binding or immunogenicity, that is, with a biological property of the protein.

The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in other embodiments, those which are within ±1 are included, and in still other embodiments, those within ±0.5 are included. In some instances, one may also identify epitopes from primary amino acid sequences on the basis of hydrophilicity. These regions are also referred to as “epitopic core regions.”

Exemplary conservative amino acid substitutions are set forth in Table 4.

TABLE 4
Conservative Amino
Acid Substitutions

| Residue | Sub |
|---|---|
| Ala | Ser |
| Arg | Lys |
| Asn | Gln, |
| | His |
| Asp | Glu |
| Cys | Ser |
| Gln | Asn |
| Glu | Asp |
| Gly | Pro |
| His | Asn, |
| | Gln |
| Ile | Leu, |
| | Val |
| Leu | Ile, Val |
| Lys | Arg, |
| | Gln, |
| | Glu |
| Met | Leu, Ile |
| Phe | Met, |
| | Leu, |
| | Tyr |
| Ser | Thr |
| Thr | Ser |
| Trp | Tyr |
| Tyr | Trp, |
| | Phe |
| Val | Ile, Leu |
| Thr | Ser |

Residue = Orignial residue
Sub = Exemplary Substitution

A skilled artisan will be able to determine suitable variants of polypeptides as set forth herein using well-known techniques. One skilled in the art may identify suitable areas of the molecule that may be changed without destroying activity by targeting regions not believed to be important for activity. The skilled artisan also will be able to identify residues and portions of the molecules that are conserved among similar polypeptides. In further embodiments, even areas that may be important for biological activity or for structure may be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the 3-dimensional structure and amino acid sequence in relation to that structure in similar polypeptides. In view of such information, one skilled in the art may predict the alignment of amino acid residues of an antibody with respect to its three dimensional structure. One skilled in the art may choose not to make radical changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using assays for IL-23 activity, (see examples below) thus yielding information regarding which amino acids can be changed and which must not be changed. In other words, based on information gathered from such routine experiments, one skilled in the art can readily determine the amino acid positions where further substitutions should be avoided either alone or in combination with other mutations.

A number of scientific publications have been devoted to the prediction of secondary structure. See, Moult, 1996, Curr. Op. in Biotech. 7:422-427; Chou et al., 1974, Biochem. 13:222-245; Chou et al., 1974, Biochemistry 113:211-222; Chou et al., 1978, Adv. Enzymol. Relat. Areas Mol. Biol. 47:45-148; Chou et al., 1979, Ann. Rev. Biochem. 47:251-276; and Chou et al., 1979, Biophys. J. 26:367-384. Moreover, computer programs are currently available to assist with predicting secondary structure. One method of predicting secondary structure is based upon homology modeling. For example, two polypeptides or proteins that have a sequence identity of greater than 30%, or similarity greater than 40% often have similar structural topologies. The recent growth of the protein structural database (PDB) has provided enhanced predictability of secondary structure, including the potential number of folds within a polypeptide's or protein's structure. See, Holm et al., 1999, Nucl. Acid. Res. 27:244-247. It has been suggested (Brenner et al., 1997, Curr. Op. Struct. Biol. 7:369-376) that there are a limited number of folds in a given polypeptide or protein and that once a critical number of structures have been resolved, structural prediction will become dramatically more accurate.

Additional methods of predicting secondary structure include “threading” (Jones, 1997, Curr. Opin. Struct. Biol. 7:377-387; Sippl et al., 1996, Structure 4:15-19), “profile analysis” (Bowie et al., 1991, Science 253:164-170; Gribskov et al., 1990, Meth. Enzymol. 183:146-159; Gribskov et al., 1987, Proc. Nat. Acad. Sci. 84:4355-4358), and “evolutionary linkage” (See, Holm, 1999, supra; and Brenner, 1997, supra).

In some embodiments, amino acid substitutions are made that: (1) reduce susceptibility to proteolysis, (2) reduce susceptibility to oxidation, (3) alter binding affinity for forming protein complexes, (4) alter ligand or antigen-binding affinities, and/or (4) confer or modify other physicochemical or functional properties on such polypeptides, such as maintaining the structure of the molecular backbone in the area of the substitution, for example, as a sheet or helical conformation; maintaining or altering the charge or hydrophobicity of the molecule at the target site, or maintaining or altering the bulkiness of a side chain.

For example, single or multiple amino acid substitutions (in certain embodiments, conservative amino acid substitutions) may be made in the naturally-occurring sequence. Substitutions can be made in that portion of the antibody that lies outside the domain(s) forming intermolecular contacts). In such embodiments, conservative amino acid substitutions can be used that do not substantially change the structural characteristics of the parent sequence (e.g., one or more replacement amino acids that do not disrupt the secondary structure that characterizes the parent or native antigen-binding protein). Examples of art-recognized polypeptide secondary and tertiary structures are described in Proteins, Structures and Molecular Principles (Creighton, Ed.), 1984, W. H. New York: Freeman and Company; Introduction to Protein Structure (Branden and Tooze, eds.), 1991, New York: Garland Publishing; and Thornton et al., 1991, Nature 354:105.

Additional variants include cysteine variants wherein one or more cysteine residues in the parent or native amino acid sequence are deleted from or substituted with another amino acid (e.g., serine). Cysteine variants are useful, inter alia when antibodies (for example) must be refolded into a biologically active conformation. Cysteine variants may have fewer cysteine residues than the native protein, and typically have an even number to minimize interactions resulting from unpaired cysteines.

The heavy and light chain variable region and CDRs that are disclosed can be used to prepare antigen-binding proteins that contain an antigen-binding region that can specifically bind to an IL-23 polypeptide. “Antigen-binding region” means a protein, or a portion of a protein, that specifically binds a specified antigen, such as the region that contains the amino acid residues that interact with an antigen and confer on the antigen-binding protein its specificity and affinity for the target antigen. An antigen-binding region may include one or more CDRs and certain antigen-binding regions also include one or more “framework” regions. For example, one or more of the CDRs listed in Table 3 can be incorporated into a molecule (e.g., a polypeptide) covalently or noncovalently to make an immunoadhesion. An immunoadhesion may incorporate the CDR(s) as part of a larger polypeptide chain, may covalently link the CDR(s) to another polypeptide chain, or may incorporate the CDR(s) noncovalently. The CDR(s) enable the immunoadhesion to bind specifically to a particular antigen of interest (e.g., an IL-23 polypeptide).

Other antigen-binding proteins include mimetics (e.g., “peptide mimetics” or “peptidomimetics”) based upon the variable regions and CDRs that are described herein. These analogs can be peptides, non-peptides or combinations of peptide and non-peptide regions. Fauchere, 1986, Adv. Drug Res. 15:29; Veber and Freidinger, 1985, TINS p. 392; and Evans et al., 1987, J. Med. Chem. 30:1229. Peptide mimetics that are structurally similar to therapeutically useful peptides may be used to produce a similar therapeutic or prophylactic effect. Such compounds are often developed with the aid of computerized molecular modeling. Generally, peptidomimetics are proteins that are structurally similar to an antigen-binding protein displaying a desired biological activity, such as the ability to bind IL-23, but peptidomimetics have one or more peptide linkages optionally replaced by a linkage selected from, for example: —CH2NH—, —CH₂S—, —CH2-CH2-, —CH—CH— (cis and trans), —COCH2-, —CH(OH)CH2-, and —CH2SO—, by methods well known in the art. Systematic substitution of one or more amino acids of a consensus sequence with a D-amino acid of the same type (e.g., D-lysine in place of L-lysine) may be used in certain embodiments to generate more stable proteins. In addition, constrained peptides comprising a consensus sequence or a substantially identical consensus sequence variation may be generated by methods known in the art (Rizo and Gierasch, 1992, Ann. Rev. Biochem. 61:387), for example, by adding internal cysteine residues capable of forming intramolecular disulfide bridges which cyclize the peptide.

Derivatives of the antigen-binding proteins that are described herein are also provided. The derivatized antigen-binding proteins can comprise any molecule or substance that imparts a desired property to the antigen-binding protein or fragment, such as increased half-life in a particular use. The derivatized antigen-binding protein can comprise, for example, a detectable (or labeling) moiety (e.g., a radioactive, colorimetric, antigenic or enzymatic molecule, a detectable bead (such as a magnetic or electrodense (e.g., gold) bead), or a molecule that binds to another molecule (e.g., biotin or Streptavidin)), a therapeutic or diagnostic moiety (e.g., a radioactive, cytotoxic, or pharmaceutically active moiety), or a molecule that increases the suitability of the antigen-binding protein for a particular use (e.g., administration to a subject, such as a human subject, or other in vivo or in vitro uses). Examples of molecules that can be used to derivatize an antigen-binding protein include albumin (e.g., human serum albumin) and polyethylene glycol (PEG). Albumin-linked and PEGylated derivatives of antigen-binding proteins can be prepared using techniques well known in the art. In one embodiment, the antigen-binding protein is conjugated or otherwise linked to transthyretin (TTR) or a TTR variant. The TTR or TTR variant can be chemically modified with, for example, a chemical selected from the group consisting of dextran, poly(n-vinyl pyrrolidone), polyethylene glycols, polypropylene glycol homopolymers, polypropylene oxide/ethylene oxide co-polymers, polyoxyethylated polyols and polyvinyl alcohols.

Other derivatives include covalent or aggregative conjugates of IL-23 antigen-binding proteins with other proteins or polypeptides, such as by expression of recombinant fusion proteins comprising heterologous polypeptides fused to the N-terminus or C-terminus of an IL-23 antigen-binding protein. For example, the conjugated peptide may be a heterologous signal (or leader) polypeptide, e.g., the yeast alpha-factor leader, or a peptide such as an epitope tag. IL-23 antigen-binding protein-containing fusion proteins can comprise peptides added to facilitate purification or identification of the IL-23 antigen-binding protein (e.g., poly-His). An IL-23 antigen-binding protein also can be linked to the FLAG peptide as described in Hopp et al., 1988, Bio/Technology 6:1204; and U.S. Pat. No. 5,011,912. The FLAG peptide is highly antigenic and provides an epitope reversibly bound by a specific monoclonal antibody (mAb), enabling rapid assay and facile purification of expressed recombinant protein. Reagents useful for preparing fusion proteins in which the FLAG peptide is fused to a given polypeptide are commercially available (Sigma, St. Louis, Mo.).

Oligomers that contain one or more IL-23 antigen-binding proteins may be employed as IL-23 antagonists. Oligomers may be in the form of covalently-linked or non-covalently-linked dimers, trimers, or higher oligomers. Oligomers comprising two or more IL-23 antigen-binding proteins are contemplated for use, with one example being a homodimer. Other oligomers include heterodimers, homotrimers, heterotrimers, homotetramers, heterotetramers, and the like. Oligomers comprising multiple IL-23-binding proteins joined via covalent or non-covalent interactions between peptide moieties fused to the IL-23 antigen-binding proteins, are also included. Such peptides may be peptide linkers (spacers), or peptides that have the property of promoting oligomerization. Among the suitable peptide linkers are those described in U.S. Pat. Nos. 4,751,180 and 4,935,233. Leucine zippers and certain polypeptides derived from antibodies are among the peptides that can promote oligomerization of IL-23 antigen-binding proteins attached thereto. Examples of leucine zipper domains suitable for producing soluble oligomeric proteins are described in WIPO Publication No. WO 94/10308; Hoppe et al., 1994, FEBS Letters 344:191; and Fanslow et al., 1994, Semin. Immunol. 6:267-278. In one approach, recombinant fusion proteins comprising an IL-23 antigen binding protein fragment or derivative fused to a leucine zipper peptide are expressed in suitable host cells, and the soluble oligomeric IL-23 antigen-binding protein fragments or derivatives that form are recovered from the culture supernatant.

Such oligomers may comprise from two to four IL-23 antigen-binding proteins. The IL-23 antigen-binding protein moieties of the oligomer may be in any of the forms described above, e.g., variants or fragments. Preferably, the oligomers comprise IL-23 antigen-binding proteins that have IL-23 binding activity. Oligomers may be prepared using polypeptides derived from immunoglobulins. Preparation of fusion proteins comprising certain heterologous polypeptides fused to various portions of antibody-derived polypeptides (including the Fc domain) has been described, e.g., by Ashkenazi et al., 1991, Proc. Natl. Acad. Sci. USA 88:10535; Byrn et al., 1990, Nature 344:677; and Hollenbaugh et al., 1992 “Construction of Immunoglobulin Fusion Proteins”, in Current Protocols in Immunology, Suppl. 4, pages 10.19.1-10.19.11.

Also included are dimers comprising two fusion proteins created by fusing an IL-23 antigen binding protein to the Fc region of an antibody. The dimer can be made by, for example, inserting a gene fusion encoding the fusion protein into an appropriate expression vector, expressing the gene fusion in host cells transformed with the recombinant expression vector, and allowing the expressed fusion protein to assemble much like antibody molecules, whereupon interchain disulfide bonds form between the Fc moieties to yield the dimer. Such Fc polypeptides include native and mutein forms of polypeptides derived from the Fc region of an antibody. Truncated forms of such polypeptides containing the hinge region that promotes dimerization also are included. Fusion proteins comprising Fc moieties (and oligomers formed therefrom) offer the advantage of facile purification by affinity chromatography over Protein A or Protein G columns. One suitable Fc polypeptide, described in WIPO Publication No. WO 93/10151 and U.S. Pat. Nos. 5,426,048 and 5,262,522, is a single chain polypeptide extending from the N-terminal hinge region to the native C-terminus of the Fc region of a human IgG1 antibody. Another useful Fc polypeptide is the Fc mutein described in U.S. Pat. No. 5,457,035, and in Baum et al., 1994, EMBO J. 13:3992-4001. The amino acid sequence of this mutein is identical to that of the native Fc sequence presented in WIPO Publication No. WO 93/10151, except that amino acid 19 has been changed from Leu to Ala, amino acid 20 has been changed from Leu to Glu, and amino acid 22 has been changed from Gly to Ala. The mutein exhibits reduced affinity for Fc receptors.

### Glycosylation

The antigen-binding protein may have a glycosylation pattern that is different or altered from that found in the native species. As is known in the art, glycosylation patterns can depend on both the sequence of the protein (e.g., the presence or absence of particular glycosylation amino acid residues, discussed below), or the host cell or organism in which the protein is produced. Particular expression systems are discussed below.

Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

Addition of glycosylation sites to the antigen-binding protein is conveniently accomplished by altering the amino acid sequence such that it contains one or more of the above-described tri-peptide sequences (for N-linked glycosylation sites). The alteration may also be made by the addition of, or substitution by, one or more serine or threonine residues to the starting sequence (for O-linked glycosylation sites). For ease, the antigen-binding protein amino acid sequence may be altered through changes at the DNA level, particularly by mutating the DNA encoding the target polypeptide at preselected bases such that codons are generated that will translate into the desired amino acids.

Another means of increasing the number of carbohydrate moieties on the antigen-binding protein is by chemical or enzymatic coupling of glycosides to the protein. These procedures are advantageous in that they do not require production of the protein in a host cell that has glycosylation capabilities for N- and O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in PCT Publication No. WO 87/05330, and in Aplin and Wriston, 1981, CRC Crit. Rev, Biochem., pp. 259-306.

Removal of carbohydrate moieties present on the starting antigen-binding protein may be accomplished chemically or enzymatically. Chemical deglycosylation requires exposure of the protein to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin et al., 1987, Arch. Biochem. Biophys. 259:52 and by Edge et al., 1981, Anal. Biochem. 118:131. Enzymatic cleavage of carbohydrate moieties on polypeptides can be achieved by the use of a variety of endo- and exo-glycosidases as described by Thotakura et al., 1987, Meth. Enzymol. 138:350. Glycosylation at potential glycosylation sites may be prevented by the use of the compound tunicamycin as described by Duskin et al., 1982, J. Biol. Chem. 257:3105. Tunicamycin blocks the formation of protein-N-glycoside linkages.

Hence, aspects include glycosylation variants of the antigen-binding proteins wherein the number and/or type of glycosylation site(s) has been altered compared to the amino acid sequences of the parent polypeptide. In certain embodiments, antigen-binding protein variants comprise a greater or a lesser number of N-linked glycosylation sites than the parent polypeptide. Substitutions that eliminate or alter this sequence will prevent addition of an N-linked carbohydrate chain present in the parent polypeptide. For example, the glycosylation can be reduced by the deletion of an Asn or by substituting the Asn with a different amino acid. Antibodies typically have a N-linked glycosylation site in the Fc region.

### Labels And Effector Groups

Antigen-binding proteins may comprise one or more labels. The term “label” or “labeling group” refers to any detectable label. In general, labels fall into a variety of classes, depending on the assay in which they are to be detected: a) isotopic labels, which may be radioactive or heavy isotopes; b) magnetic labels (e.g., magnetic particles); c) redox active moieties; d) optical dyes; enzymatic groups (e.g., horseradish peroxidase, (3-galactosidase, luciferase, alkaline phosphatase); e) biotinylated groups; and f) predetermined polypeptide epitopes recognized by a secondary reporter (e.g., leucine zipper pair sequences, binding sites for secondary antibodies, metal binding domains, epitope tags, and the like). In some embodiments, the labeling group is coupled to the antigen-binding protein via spacer arms of various lengths to reduce potential steric hindrance. Various methods for labeling proteins are known in the art. Examples of suitable labeling groups include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent groups (e.g., FITC, rhodamine, lanthanide phosphors), enzymatic groups (e.g., horseradish peroxidase, (3-galactosidase, luciferase, alkaline phosphatase), chemiluminescent groups, biotinyl groups, or predetermined polypeptide epitopes recognized by a secondary reporter (e.g., leucine zipper pair sequences, binding sites for secondary antibodies, metal binding domains, epitope tags). In some embodiments, the labeling group is coupled to the antigen-binding protein via spacer arms of various lengths to reduce potential steric hindrance. Various methods for labeling proteins are known in the art and may be used as is seen fit.

The term “effector group” means any group coupled to an antigen-binding protein that acts as a cytotoxic agent. Examples for suitable effector groups are radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I). Other suitable groups include toxins, therapeutic groups, or chemotherapeutic groups. Examples of suitable groups include calicheamicin, auristatins, geldanamycin and maytansine. In some embodiments, the effector group is coupled to the antigen binding protein via spacer arms of various lengths to reduce potential steric hindrance.

### Polynucleotides Encoding IL-23 Antigen-Binding Proteins

Polynucleotides that encode the antigen-binding proteins described herein, or portions thereof, are also provided, including polynucleotides encoding one or both chains of an antibody, or a fragment, derivative, mutein, or variant thereof, polynucleotides encoding heavy chain variable regions or only CDRs, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing. The polynucleotides can be any length. They can be, for example, 5, 10, 15, 20, 25, 30, 35, 40, 45, 50, 75, 85, 95, 100, 125, 150, 175, 200, 250, 300, 350, 400, 450, 500, 750, 1,000, 1,500, 3,000, 5,000 or more nucleic acids in length, including all values in between, and/or can comprise one or more additional sequences, for example, regulatory sequences, and/or be part of a larger polynucleotide, for example, a vector. The polynucleotides can be single-stranded or double-stranded and can comprise RNA and/or DNA nucleic acids and artificial variants thereof (e.g., peptide nucleic acids).

Polynucleotides encoding certain antigen-binding proteins, or portions thereof (e.g., full length antibody, heavy or light chain, variable domain, or a CDRH1, CDRH2, CDRH3, CDRL1, CDRL2, or CDRL3) may be isolated from B-cells of mice that have been immunized with IL-23 or an immunogenic fragment thereof. The polynucleotide may be isolated by conventional procedures such as polymerase chain reaction (PCR). Phage display is another example of a known technique whereby derivatives of antibodies and other antigen-binding proteins may be prepared. In one approach, polypeptides that are components of an antigen-binding protein of interest are expressed in any suitable recombinant expression system, and the expressed polypeptides are allowed to assemble to form antigen-binding protein molecules. Phage display is also used to derive antigen binding proteins having different properties (i.e., varying affinities for the antigen to which they bind) via chain shuffling, see Marks et al., 1992, BioTechnology 10:779.

Due to the degeneracy of the genetic code, each of the polypeptide sequences depicted herein are also encoded by a large number of other polynucleotide sequences besides those provided. For example, heavy chain variable domains provided herein in may be encoded by polynucleotide sequences SEQ ID NOs: 32, 35, 37, 39, 41, 43, 45, 47, 49, 51, 53, 55, 57, or 59. Light chain variable domains may be encoded by polynucleotide sequences SEQ ID NOs:2, 5, 6, 8, 10, 12, 14, 16, 18, 20, 22, 24, 26, or 28. One of ordinary skill in the art will appreciate that the present disclosure thus provides adequate written description and enablement for each degenerate nucleotide sequence encoding each antigen-binding protein.

An aspect further provides polynucleotides that hybridize to other polynucleotide molecules under particular hybridization conditions. Methods for hybridizing nucleic acids, basic parameters affecting the choice of hybridization conditions and guidance for devising suitable conditions are well-known in the art. See, e.g., Sambrook, Fritsch, and Maniatis (2001, Molecular Cloning: A Laboratory Manual, Cold Spring Harbor Laboratory Press, Cold Spring Harbor, N.Y. and Current Protocols in Molecular Biology, 1995, Ausubel et al., eds., John Wiley & Sons, Inc. As defined herein, a moderately stringent hybridization condition uses a prewashing solution containing 5× sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C., in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that polynucleotides comprising nucleic acid sequences that are at least 65%, 70%, 75%, 80%, 85%, 90%, 91%, 92, 93%, 94%, 95%, 96%, 97%, 98% or 99% identical to each other, including all values in between, typically remain hybridized to each other.

Changes can be introduced by mutation into a polynucleotide, thereby leading to changes in the amino acid sequence of a polypeptide (e.g., an antigen-binding protein or antigen-binding protein derivative) that it encodes. Mutations can be introduced using any technique known in the art, such as site-directed mutagenesis and random mutagenesis. Mutant polypeptides can be expressed and selected for a desired property. Mutations can be introduced into a polynucleotide without significantly altering the biological activity of a polypeptide that it encodes. For example, substitutions at non-essential amino acid residues. Alternatively, one or more mutations can be introduced into a polynucleotide that selectively change the biological activity of a polypeptide that it encodes. For example, the mutation can quantitatively or qualitatively change the biological activity, such as increasing, reducing or eliminating the activity and changing the antigen specificity of an antigen binding protein.

Another aspect provides polynucleotides that are suitable for use as primers or hybridization probes for the detection of nucleic acid sequences. A polynucleotide can comprise only a portion of a nucleic acid sequence encoding a full-length polypeptide, for example, a fragment that can be used as a probe or primer or a fragment encoding an active portion (e.g., an IL-23 binding portion) of a polypeptide. Probes based on the sequence of a nucleic acid can be used to detect the nucleic acid or similar nucleic acids, for example, transcripts encoding a polypeptide. The probe can comprise a label group, e.g., a radioisotope, a fluorescent compound, an enzyme, or an enzyme co-factor. Such probes can be used to identify a cell that expresses the polypeptide.

### Methods of Expressing Antigen-Binding Proteins

The antigen-binding proteins provided herein may be prepared by any of a number of conventional techniques. For example, IL-23 antigen-binding proteins may be produced by recombinant expression systems, using any technique known in the art. See, e.g., Monoclonal Antibodies, Hybridomas: A New Dimension in Biological Analyses, Kennet et al. (eds.) Plenum Press, New York (1980); and Antibodies: A Laboratory Manual, Harlow and Lane (eds.), Cold Spring Harbor Laboratory Press, Cold Spring Harbor, N.Y. (1988).

Expression systems and constructs in the form of plasmids, expression vectors, transcription or expression cassettes that comprise at least one polynucleotide as described above are also provided herein, as well host cells comprising such expression systems or constructs. As used herein, “vector” means any molecule or entity (e.g., nucleic acid, plasmid, bacteriophage or virus) suitable for use to transfer protein coding information into a host cell. Examples of vectors include, but are not limited to, plasmids, viral vectors, non-episomal mammalian vectors and expression vectors, for example, recombinant expression vectors. Expression vectors, such as recombinant expression vectors, are useful for transformation of a host cell and contain nucleic acid sequences that direct and/or control (in conjunction with the host cell) expression of one or more heterologous coding regions operatively linked thereto. An expression construct may include, but is not limited to, sequences that affect or control transcription, translation, and, if introns are present, affect RNA splicing of a coding region operably linked thereto. “Operably linked” means that the components to which the term is applied are in a relationship that allows them to carry out their inherent functions. For example, a control sequence, e.g., a promoter, in a vector that is “operably linked” to a protein coding sequence are arranged such that normal activity of the control sequence leads to transcription of the protein coding sequence resulting in recombinant expression of the encoded protein.

Another aspect provides host cells into which an expression vector, such as a recombinant expression vector, has been introduced. A host cell can be any prokaryotic cell (for example, E. coli) or eukaryotic cell (for example, yeast, insect, or mammalian cells (e.g., CHO cells)). Vector DNA can be introduced into prokaryotic or eukaryotic cells via conventional transformation or transfection techniques. For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced polynucleotide can be identified by drug selection (e.g., cells that have incorporated the selectable marker gene will survive, while the other cells die), among other methods.

Antigen-binding proteins can be expressed in hybridoma cell lines (e.g., in particular antibodies may be expressed in hybridomas) or in cell lines other than hybridomas. Expression constructs encoding the antigen-binding proteins can be used to transform a mammalian, insect or microbial host cell. Transformation can be performed using any known method for introducing polynucleotides into a host cell, including, for example packaging the polynucleotide in a virus or bacteriophage and transducing a host cell with the construct by transfection procedures known in the art, as exemplified by U.S. Pat. Nos. 4,399,216; 4,912,040; 4,740,461; 4,959,455. The optimal transformation procedure used will depend upon which type of host cell is being transformed. Methods for introduction of heterologous polynucleotides into mammalian cells are well known in the art and include, but are not limited to, dextran-mediated transfection, calcium phosphate precipitation, polybrene mediated transfection, protoplast fusion, electroporation, encapsulation of the polynucleotide(s) in liposomes, mixing nucleic acid with positively-charged lipids, and direct microinjection of the DNA into nuclei.

Recombinant expression constructs typically comprise a polynucleotide encoding a polypeptide. The polypeptide may comprise one or more of the following: one or more CDRs such as provided herein; a light chain variable region; a heavy chain variable region; a light chain constant region; a heavy chain constant region (e.g., CH1, CH2 and/or CH3); and/or another scaffold portion of an IL-23 antigen-binding protein. These nucleic acid sequences are inserted into an appropriate expression vector using standard ligation techniques. In one embodiment, the heavy or light chain constant region is appended to the C-terminus of a heavy or light chain variable region provided herein and is ligated into an expression vector. The vector is typically selected to be functional in the particular host cell employed (i.e., the vector is compatible with the host cell machinery, permitting amplification and/or expression of the gene can occur). In some embodiments, vectors are used that employ protein-fragment complementation assays using protein reporters, such as dihydrofolate reductase (see, for example, U.S. Pat. No. 6,270,964). Suitable expression vectors can be purchased, for example, from Invitrogen Life Technologies (Carlsbad, Calif.) or BD Biosciences (San Jose, Calif.). Other useful vectors for cloning and expressing the antibodies and fragments include those described in Bianchi and McGrew, 2003, Biotech. Biotechnol. Bioeng. 84:439-44. Additional suitable expression vectors are discussed, for example, in Methods Enzymol., vol. 185 (D. V. Goeddel, ed.), 1990, New York: Academic Press.

Typically, expression vectors used in any of the host cells will contain sequences for plasmid maintenance and for cloning and expression of exogenous nucleotide sequences. Such sequences, collectively referred to as “flanking sequences” in certain embodiments will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the polynucleotide encoding the polypeptide to be expressed, and a selectable marker element. The expression vectors that are provided may be constructed from a starting vector such as a commercially available vector. Such vectors may or may not contain all of the desired flanking sequences. Where one or more of the flanking sequences described herein are not already present in the vector, they may be individually obtained and ligated into the vector. Methods used for obtaining each of the flanking sequences are well known to one skilled in the art.

Optionally, the vector may contain a “tag”-encoding sequence, i.e., an oligonucleotide molecule located at the 5′ or 3′ end of the IL-23 antigen-binding protein coding sequence; the oligonucleotide sequence encodes polyHis (such as hexaHis; SEQ ID NO:152), or another “tag” such as FLAG®, HA (hemagluttinin influenza virus), or myc, for which commercially available antibodies exist. This tag is typically fused to the polypeptide upon expression of the polypeptide, and can serve as a means for affinity purification or detection of the IL-23 antigen-binding protein from the host cell. Affinity purification can be accomplished, for example, by column chromatography using antibodies against the tag as an affinity matrix. Optionally, the tag can subsequently be removed from the purified IL-23 antigen-binding protein by various means such as using certain peptidases for cleavage.

Flanking sequences may be homologous (i.e., from the same species and/or strain as the host cell), heterologous (i.e., from a species other than the host cell species or strain), hybrid (i.e., a combination of flanking sequences from more than one source), synthetic or native. As such, the source of a flanking sequence may be any prokaryotic or eukaryotic organism, any vertebrate or invertebrate organism, or any plant, provided that the flanking sequence is functional in, and can be activated by, the host cell machinery.

Flanking sequences useful in the vectors may be obtained by any of several methods well known in the art. Typically, flanking sequences useful herein will have been previously identified by mapping and/or by restriction endonuclease digestion and can thus be isolated from the proper tissue source using the appropriate restriction endonucleases. In some cases, the full nucleotide sequence of a flanking sequence may be known. Here, the flanking sequence may be synthesized using the methods described herein for nucleic acid synthesis or cloning.

Whether all or only a portion of the flanking sequence is known, it may be obtained using polymerase chain reaction (PCR) and/or by screening a genomic library with a suitable probe such as an oligonucleotide and/or flanking sequence fragment from the same or another species. Where the flanking sequence is not known, a fragment of DNA containing a flanking sequence may be isolated from a larger piece of DNA that may contain, for example, a coding sequence or even another gene or genes. Isolation may be accomplished by restriction endonuclease digestion to produce the proper DNA fragment followed by isolation using agarose gel purification, Qiagen® column chromatography (Qiagen, Chatsworth, Calif.), or other methods known to the skilled artisan. The selection of suitable enzymes to accomplish this purpose will be readily apparent to one of ordinary skill in the art.

An origin of replication is typically a part of those prokaryotic expression vectors purchased commercially, and the origin aids in the amplification of the vector in a host cell. If the vector of choice does not contain an origin of replication site, one may be chemically synthesized based on a known sequence, and ligated into the vector. For example, the origin of replication from the plasmid pBR322 (New England Biolabs, Beverly, Mass.) is suitable for most gram-negative bacteria, and various viral origins (e.g., SV40, polyoma, adenovirus, vesicular stomatitis virus (VSV), or papillomaviruses such as HPV or BPV) are useful for cloning vectors in mammalian cells. Generally, the origin of replication component is not needed for mammalian expression vectors (for example, the SV40 origin is often used only because it also contains the virus early promoter).

A transcription termination sequence is typically located 3′ to the end of a polypeptide coding region and serves to terminate transcription. Usually, a transcription termination sequence in prokaryotic cells is a G-C rich fragment followed by a poly-T sequence. While the sequence is easily cloned from a library or even purchased commercially as part of a vector, it can also be readily synthesized using methods for nucleic acid synthesis such as those described herein.

A selectable marker gene encodes a protein necessary for the survival and growth of a host cell grown in a selective culture medium. Typical selection marker genes encode proteins that (a) confer resistance to antibiotics or other toxins, e.g., ampicillin, tetracycline, or kanamycin for prokaryotic host cells; (b) complement auxotrophic deficiencies of the cell; or (c) supply critical nutrients not available from complex or defined media. Specific selectable markers are the kanamycin resistance gene, the ampicillin resistance gene, and the tetracycline resistance gene. Advantageously, a neomycin resistance gene may also be used for selection in both prokaryotic and eukaryotic host cells.

Other selectable genes may be used to amplify the gene that will be expressed. Amplification is the process wherein genes that are required for production of a protein critical for growth or cell survival are reiterated in tandem within the chromosomes of successive generations of recombinant cells. Examples of suitable selectable markers for mammalian cells include dihydrofolate reductase (DHFR) and promoterless thymidine kinase genes. Mammalian cell transformants are placed under selection pressure wherein only the transformants are uniquely adapted to survive by virtue of the selectable gene present in the vector. Selection pressure is imposed by culturing the transformed cells under conditions in which the concentration of selection agent in the medium is successively increased, thereby leading to the amplification of both the selectable gene and the DNA that encodes another gene, such as an antigen-binding protein that binds to IL-23. As a result, increased quantities of a polypeptide such as an antigen-binding protein are synthesized from the amplified DNA.

A ribosome-binding site is usually necessary for translation initiation of rnRNA and is characterized by a Shine-Dalgarno sequence (prokaryotes) or a Kozak sequence (eukaryotes). The element is typically located 3′ to the promoter and 5′ to the coding sequence of the polypeptide to be expressed.

In some cases, such as where glycosylation is desired in a eukaryotic host cell expression system, one may manipulate the various pre- or pro-sequences to improve glycosylation or yield. For example, one may alter the peptidase cleavage site of a particular signal peptide, or add prosequences, which also may affect glycosylation. The final protein product may have, in the −1 position (relative to the first amino acid of the mature protein), one or more additional amino acids incident to expression, which may not have been totally removed. For example, the final protein product may have one or two amino acid residues found in the peptidase cleavage site, attached to the amino-terminus. Alternatively, use of some enzyme cleavage sites may result in a slightly truncated form of the desired polypeptide, if the enzyme cuts at such area within the mature polypeptide.

Expression and cloning will typically contain a promoter that is recognized by the host organism and operably linked to the molecule encoding an IL-23 antigen-binding protein. Promoters are untranscribed sequences located upstream (i.e., 5′) to the start codon of a structural gene (generally within about 100 to 1000 bp) that control transcription of the structural gene. Promoters are conventionally grouped into one of two classes: inducible promoters and constitutive promoters. Inducible promoters initiate increased levels of transcription from DNA under their control in response to some change in culture conditions, such as the presence or absence of a nutrient or a change in temperature. Constitutive promoters, on the other hand, uniformly transcribe a gene to which they are operably linked, that is, with little or no control over gene expression. A large number of promoters, recognized by a variety of potential host cells, are well known. A suitable promoter is operably linked to the DNA encoding a heavy chain variable region or a light chain variable region of an IL-23 antigen binding protein by removing the promoter from the source DNA by restriction enzyme digestion and inserting the desired promoter sequence into the vector.

Suitable promoters for use with yeast hosts are also well known in the art. Yeast enhancers are advantageously used with yeast promoters. Suitable promoters for use with mammalian host cells are well known and include, but are not limited to, those obtained from the genomes of viruses such as polyoma virus, fowlpox virus, adenovirus (such as Adenovirus 2), bovine papilloma virus, avian sarcoma virus, cytomegalovirus, retroviruses, hepatitis-B virus, and Simian Virus 40 (SV40). Other suitable mammalian promoters include heterologous mammalian promoters, for example, heat-shock promoters and the actin promoter.

Additional promoters of interest include, but are not limited to: SV40 early promoter (Benoist and Chambon, 1981, Nature 290:304-310); CMV promoter (Thornsen et al., 1984, Proc. Natl. Acad. U.S.A. 81:659-663); the promoter contained in the 3′ long terminal repeat of Rous sarcoma virus (Yamamoto et al., 1980, Cell 22:787-797); herpes thymidine kinase promoter (Wagner et al., 1981, Proc. Natl. Acad. Sci. U.S.A. 78:1444-1445); promoter and regulatory sequences from the metallothionine gene (Prinster et al., 1982, Nature 296:39-42); and prokaryotic promoters such as the beta-lactamase promoter (Villa-Kamaroff et al., 1978, Proc. Natl. Acad. Sci. U.S.A. 75:3727-3731); or the tac promoter (DeBoer et al., 1983, Proc. Natl. Acad. Sci. U.S.A. 80:21-25). Also of interest are the following animal transcriptional control regions, which exhibit tissue specificity and have been utilized in transgenic animals: the elastase I gene control region that is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); the insulin gene control region that is active in pancreatic beta cells (Hanahan, 1985, Nature 315:115-122); the immunoglobulin gene control region that is active in lymphoid cells (Grosschedl et al., 1984, Cell 38:647-658; Adames et al., 1985, Nature 318:533-538; Alexander et al., 1987, Mol. Cell. Biol. 7:1436-1444); the mouse mammary tumor virus control region that is active in testicular, breast, lymphoid and mast cells (Leder et al., 1986, Cell 45:485-495); the albumin gene control region that is active in liver (Pinkert et al., 1987, Genes and Devel. 1:268-276); the alpha-feto-protein gene control region that is active in liver (Krumlauf et al., 1985, Mol. Cell. Biol. 5:1639-1648; Hammer et al., 1987, Science 253:53-58); the alpha 1-antitrypsin gene control region that is active in liver (Kelsey et al., 1987, Genes and Devel. 1:161-171); the beta-globin gene control region that is active in myeloid cells (Mogram et al., 1985, Nature 315:338-340; Kollias et al., 1986, Cell 46:89-94); the myelin basic protein gene control region that is active in oligodendrocyte cells in the brain (Readhead et al., 1987, Cell 48:703-712); the myosin light chain-2 gene control region that is active in skeletal muscle (Sani, 1985, Nature 314:283-286); and the gonadotropic releasing hormone gene control region that is active in the hypothalamus (Mason et al., 1986, Science 234:1372-1378).

An enhancer sequence may be inserted into the vector to increase transcription by higher eukaryotes. Enhancers are cis-acting elements of DNA, usually about 10-300 bp in length, that act on the promoter to increase transcription. Enhancers are relatively orientation and position independent, having been found at positions both 5′ and 3′ to the transcription unit. Several enhancer sequences available from mammalian genes are known (e.g., globin, elastase, albumin, alpha-feto-protein and insulin). Typically, however, an enhancer from a virus is used. The SV40 enhancer, the cytomegalovirus early promoter enhancer, the polyoma enhancer, and adenovirus enhancers known in the art are exemplary enhancing elements for the activation of eukaryotic promoters. While an enhancer may be positioned in the vector either 5′ or 3′ to a coding sequence, it is typically located at a site 5′ from the promoter. A sequence encoding an appropriate native or heterologous signal sequence (leader sequence or signal peptide) can be incorporated into an expression vector, to promote extracellular secretion of the antibody. The choice of signal peptide or leader depends on the type of host cells in which the antibody is to be produced, and a heterologous signal sequence can replace the native signal sequence. Examples of signal peptides that are functional in mammalian host cells include the following: the signal sequence for interleukin-7 described in U.S. Pat. No. 4,965,195; the signal sequence for interleukin-2 receptor described in Cosman et al., 1984, Nature 312:768; the interleukin-4 receptor signal peptide described in EP Patent No. 0367 566; the type I interleukin-1 receptor signal peptide described in U.S. Pat. No. 4,968,607; the type II interleukin-1 receptor signal peptide described in EP Patent No. 0 460 846.

After the vector has been constructed, the completed vector may be inserted into a suitable host cell for amplification and/or polypeptide expression. The transformation of an expression vector for an antigen-binding protein into a selected host cell may be accomplished by well-known methods including transfection, infection, calcium phosphate co-precipitation, electroporation, microinjection, lipofection, DEAE-dextran mediated transfection, or other known techniques. The method selected will in part be a function of the type of host cell to be used. These methods and other suitable methods are well known to the skilled artisan, and are set forth, for example, in Sambrook et al., Molecular Cloning: A Laboratory Manual, 3rd ed., Cold Spring Harbor Laboratory Press, Cold Spring Harbor, N.Y. (2001).

A host cell, when cultured under appropriate conditions, synthesizes protein that can be subsequently collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule.

Mammalian cell lines available as hosts for expression are well known in the art and include, but are not limited to, immortalized cell lines available from the American Type Culture Collection (ATCC), including but not limited to Chinese hamster ovary (CHO) cells, HeLa cells, baby hamster kidney (BHK) cells, monkey kidney cells (COS), human hepatocellular carcinoma cells (e.g., Hep G2), and a number of other cell lines. In certain embodiments, cell lines may be selected through determining which cell lines have high expression levels and constitutively produce antigen-binding proteins with IL-23 binding properties. In another embodiment, a cell line from the B cell lineage that does not make its own antibody but has a capacity to make and secrete a heterologous antibody can be also selected.

### Use of Human IL-23 Antigen-Binding Proteins for Diagnostic and Therapeutic Purposes

Antigen-binding proteins are useful for detecting IL-23 in biological samples and identification of cells or tissues that produce IL-23. Antigen-binding proteins that specifically bind to IL-23 may be used in diagnosis and/or treatment of diseases related to IL-23 in a patient in need thereof. For one, the IL-23 antigen-binding proteins can be used in diagnostic assays, e.g., binding assays to detect and/or quantify IL-23 expressed in blood, serum, cells or tissue. In addition, IL-23 antigen-binding proteins can be used to reduce, inhibit, interfere with or modulate one or more biological activities of IL-23 in a cell or tissue. Thus antigen-binding proteins that bind to IL-23 may have therapeutic use in ameliorating diseases related to IL-23.

### Indications

The disclosure also relates to the use of IL-23 antigen-binding proteins in the prevention or therapeutic treatment of medical disorders, such as those disclosed herein. The IL-23 antigen-binding proteins are useful to treat a variety of conditions in which IL-23 is associated with or plays a role in contributing to the underlying disease or disorder or otherwise contributes to a negative symptom.

Conditions effectively treated by IL-23 antigen-binding proteins play a role in the inflammatory response. Such inflammatory disorders include periodontal disease; lung disorders such as asthma; skin disorders such as psoriasis, atopic dermatitis, contact dermatitis; rheumatic disorders such as rheumatoid arthritis, progressive systemic sclerosis (scleroderma); systemic lupus erythematosus; spondyloarthritis including ankylosing spondylitis, psoriatic arthritis, enteropathic arthritis and reactive arthritis. Also contemplated is uveitis including Vogt-Koyanagi-Harada disease, idiopathic anterior and posterior uveitis, and uveitis associated with spondyloarthritis. Use of IL-23 antigen-binding proteins is also contemplated for the treatment of autoimmune disorders including multiple sclerosis; autoimmune myocarditis; type 1 diabetes and autoimmune thyroiditis.

Degenerative conditions of the gastrointestinal system are treatable or preventable with IL-23 antigen-binding proteins. Such gastrointestinal disorders including inflammatory bowel disease: Crohn's disease, ulcerative colitis and Celiac disease.

Also included are use of IL-23 antigen-binding proteins in treatments for graft-versus-host disease, and complications such as graft rejection, resulting from solid organ transplantation, such as heart, liver, skin, kidney, lung or other transplants, including bone marrow transplants.

Also provided herein are methods for using IL-23 antigen-binding proteins to treat various oncologic disorders including various forms of cancer including colon, stomach, prostate, renal cell, cervical and ovarian cancers, and lung cancer (SCLC and NSCLC). Also included are solid tumors, including sarcoma, osteosarcoma, and carcinoma, such as adenocarcinoma and squamous cell carcinoma, esophageal cancer, gastric cancer, gall bladder carcinoma, leukemia, including acute myelogenous leukemia, chronic myelogenous leukemia, myeloid leukemia, chronic or acute lymphoblastic leukemia and hairy cell leukemia, and multiple myeloma.

### Detection Methods

The antigen-binding proteins of the described can be used for diagnostic purposes to detect, diagnose, or monitor diseases and/or conditions associated with IL-23. Examples of methods useful in the detection of the presence of IL-23 include immunoassays, such as the enzyme linked immunosorbent assay (ELISA) and the radioimmunoassay (RIA).

For diagnostic applications, the antigen-binding protein typically will be labeled with a detectable labeling group. Suitable labeling groups include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent groups (e.g., FITC, rhodamine, lanthanide phosphors), enzymatic groups (e.g., horseradish peroxidase, p-galactosidase, luciferase, alkaline phosphatase), chemiluminescent groups, biotinyl groups, or predetermined polypeptide epitopes recognized by a secondary reporter (e.g., leucine zipper pair sequences, binding sites for secondary antibodies, metal binding domains, epitope tags). In some embodiments, the labeling group is coupled to the antigen-binding protein via spacer arms of various lengths to reduce potential steric hindrance. Various methods for labeling proteins are known in the art and may be used.

Other diagnostic methods are provided for identifying a cell or cells that express IL-23. In a specific embodiment, the antigen-binding protein is labeled with a labeling group and the binding of the labeled antigen-binding protein to IL-23 is detected. In a further specific embodiment, the binding of the antigen-binding protein to IL-23 is detected in vivo. In a further specific embodiment, the IL-23 antigen-binding protein is isolated and measured using techniques known in the art. See, for example, Harlow and Lane, 1988, Antibodies: A Laboratory Manual, New York: Cold Spring Harbor (ed. 1991 and periodic supplements); John E. Coligan, ed., 1993, Current Protocols In Immunology New York: John Wiley & Sons.

Other methods provide for detecting the presence of a test molecule that competes for binding to IL-23 with the antigen-binding proteins provided. An example of one such assay involves detecting the amount of free antigen-binding protein in a solution containing an amount of IL-23 in the presence or absence of the test molecule. An increase in the amount of free antigen-binding protein (i.e., the antigen-binding protein not bound to IL-23) would indicate that the test molecule is capable of competing for IL-23 binding with the antigen-binding protein. In one embodiment, the antigen-binding protein is labeled with a labeling group. Alternatively, the test molecule is labeled and the amount of free test molecule is monitored in the presence and absence of an antigen-binding protein.

### Methods of Treatment: Pharmaceutical Formulations, Routes of Administration

Pharmaceutical compositions that comprise a therapeutically effective amount of one or a plurality of the antigen binding proteins and a pharmaceutically acceptable excipient, diluent, carrier, solubilizer, emulsifier, preservative, and/or adjuvant are provided. In addition, methods of treating a patient by administering such pharmaceutical composition are included. The term “patient” includes human patients. The terms “treat” and “treatment” encompass alleviation or prevention of at least one symptom or other aspect of a disorder, or reduction of disease severity, and the like. The term “therapeutically effective amount” or “effective amount” refers to the amount of an IL-23 antigen binding protein determined to produce any therapeutic response in a mammal. Such therapeutically effective amounts are readily ascertained by one of ordinary skill in the art.

The treatment methods can, for example, have a generally beneficial effect on the subject, e.g., they can increase the subject's expected longevity. Alternatively, the methods can, for example, treat, prevent, cure, relieve, or ameliorate (“treat”) a disease, disorder, condition, or illness (“a condition”). Some embodiments provide a method of treating a condition in a subject comprising administering the pharmaceutical composition comprising an specific antibody to the subject, wherein the condition is treatable by reducing the activity (partially or fully) of IL-23 in the subject. Treating encompasses both therapeutic administration (i.e., administration when signs and symptoms of the disease or condition are apparent), as well as treating to induce remission and/or maintain remission. Accordingly, the severity of the disease or condition can be reduced (partially, significantly or completely). Proactive treating encompasses the forms of treating described above and prophylactic or maintenance therapy (i.e., administration when the disease or condition is quiescent), or the signs and symptoms can be prevented or delayed (delayed onset, prolonged remission, or quiescence).

It is understood that the methods of treating the diseases described herein would administer an effective amount of an anti-IL-23 antibody. Depending on the indication to be treated, a therapeutically effective amount is sufficient to cause a reduction in at least one symptom of the targeted pathological condition by at least about 5%, 10%, 15%, 20%, 25%, 30%, 35%, 40%, 45%, 50%, 55%, 60%, 65%, 70%, 75%, 80%, 85%, 90%, 95%, or more, relative to untreated subjects.

An antigen binding protein need not effect a complete cure, or eradicate every symptom or manifestation of a disease, to constitute a viable therapeutic agent. As is recognized in the pertinent field, drugs employed as therapeutic agents may reduce the severity of a given disease state, but need not abolish every manifestation of the disease to be regarded as useful therapeutic agents. Similarly, a prophylactically administered treatment need not be completely effective in preventing the onset of a condition in order to constitute a viable prophylactic agent. Simply reducing the impact of a disease (for example, by reducing the number or severity of its symptoms, or by increasing the effectiveness of another treatment, or by producing another beneficial effect), or reducing the likelihood that the disease will occur or worsen in a subject, is sufficient. Certain methods provided herein comprise administering to a patient an IL-23 antagonist (such as the antigen binding proteins disclosed herein) in an amount and for a time sufficient to induce a sustained improvement over baseline of an indicator that reflects the severity of the particular disorder.

As is understood in the pertinent field, pharmaceutical compositions comprising the molecules of the invention are administered to a patient in a manner appropriate to the indication. Pharmaceutical compositions may be administered by any suitable technique, including but not limited to, parenterally, topically, or by inhalation. If injected, the pharmaceutical composition can be administered, for example, via intra-articular, intravenous, intramuscular, intralesional, intraperitoneal or subcutaneous routes, by bolus injection, or continuous infusion. Localized administration, e.g., at a site of disease or injury is contemplated, as are transdermal delivery and sustained release from implants. Delivery by inhalation includes, for example, nasal or oral inhalation, use of a nebulizer, inhalation of the antagonist in aerosol form, and the like. Other alternatives include eye drops; oral preparations including pills, syrups, lozenges or chewing gum; and topical preparations such as lotions, gels, sprays, and ointments.

Use of antigen binding proteins in ex vivo procedures also is contemplated. For example, a patient's blood or other bodily fluid may be contacted with an antigen binding protein that binds IL-23 ex vivo. The antigen binding protein may be bound to a suitable insoluble matrix or solid support material.

Advantageously, antigen binding proteins are administered in the form of a composition comprising one or more additional components such as a physiologically acceptable carrier, excipient or diluent. Optionally, the composition additionally comprises one or more physiologically active agents for combination therapy. A pharmaceutical composition may comprise an IL-23 antigen binding protein together with one or more substances selected from the group consisting of a buffer, an antioxidant such as ascorbic acid, a low molecular weight polypeptide (such as those having fewer than 10 amino acids), a protein, an amino acid, a carbohydrate such as glucose, sucrose or dextrins, a chelating agent such as EDTA, glutathione, a stabilizer, and an excipient. Neutral buffered saline or saline mixed with conspecific serum albumin are examples of appropriate diluents. In accordance with appropriate industry standards, preservatives such as benzyl alcohol may also be added. The composition may be formulated as a lyophilizate using appropriate excipient solutions (e.g., sucrose) as diluents. Suitable components are nontoxic to recipients at the dosages and concentrations employed. Further examples of components that may be employed in pharmaceutical formulations are presented in any Remington's Pharmaceutical Sciences including the 21st Ed. (2005), Mack Publishing Company, Easton, Pa.

Kits for use by medical practitioners include an IL-23 antigen binding protein and a label or other instructions for use in treating any of the conditions discussed herein. In one embodiment, the kit includes a sterile preparation of one or more IL-23 binding antigen binding proteins, which may be in the form of a composition as disclosed above, and may be in one or more vials.

Dosages and the frequency of administration may vary according to such factors as the route of administration, the particular antigen binding proteins employed, the nature and severity of the disease to be treated, whether the condition is acute or chronic, and the size and general condition of the subject. Appropriate dosages can be determined by procedures known in the pertinent art, e.g., in clinical trials that may involve dose escalation studies.

A typical dosage may range from about 0.1 pg/kg to up to about 30 mg/kg or more, depending on the factors mentioned above. In specific embodiments, the dosage may range from 0.1 pg/kg up to about 30 mg/kg, optionally from 1 pg/kg up to about 30 mg/kg, optionally from 10 pg/kg up 20 to about 10 mg/kg, optionally from about 0.1 mg/kg to 5 mg/kg, or optionally from about 0.3 mg/kg to 3 mg/kg.

Dosing frequency will depend upon the pharmacokinetic parameters of the particular human IL-23 antigen binding protein in the formulation used. Typically, a clinician administers the composition until a dosage is reached that achieves the desired effect. The composition may therefore be administered as a single dose, or as two or more doses (which may or may not contain the same amount of the desired molecule) over time, or as a continuous infusion via an implantation device or catheter. Appropriate dosages may be ascertained through use of appropriate dose-response data. An IL-23 antigen binding protein of the invention may be administered, for example, once or more than once, e.g., at regular intervals over a period of time. In particular embodiments, an IL-23 antigen binding protein is administered over a period of at least a month or more, e.g., for one, two, or three months or even indefinitely. For treating chronic conditions, long-term treatment is generally most effective. For treating acute conditions, however, administration for shorter periods, e.g., from one to six weeks, may be sufficient. In general, the antigen binding protein is administered until the patient manifests a medically relevant degree of improvement over baseline for the chosen indicator or indicators.

It is contemplated that an IL-23 antigen binding protein be administered to the patient in an amount and for a time sufficient to induce an improvement, preferably a sustained improvement, in at least one indicator that reflects the severity of the disorder that is being treated. Various indicators that reflect the extent of the patient's illness, disease or condition may be measured for determining whether the amount and time of the treatment is sufficient. Such indicators include, for example, clinically recognized indicators of disease severity, symptoms, or manifestations of the disorder in question. In one embodiment, an improvement is considered to be sustained if the subject exhibits the improvement on at least two occasions separated by two to four weeks. The degree of improvement generally is determined by a physician, who may make this determination based on signs, symptoms, biopsies, or other test results, and who may also employ questionnaires that are administered to the subject, such as quality-of-life questionnaires developed for a given disease.

Treatment of a subject with an IL-23 specific antibody may be given in an amount and/or at sufficient interval to achieve and/or maintain a certain quantity of IL-23-specific antibody per volume of serum, using, for example, an assay as described herein. For example, the heterodimer-specific antibody is given to achieve a serum concentration of from 12.5 ng/ml to 1,000 ng/ml. In one embodiment, the heterodimer-specific antibody is given to achieve at least 12.5 ng/ml, 25 ng/ml, 50 ng/ml, 60 ng/ml, 70 ng/ml, 75 ng/ml, 80 ng/ml, 85 ng/ml, 90 ng/ml, 95 ng/ml, 100 ng/ml, 150 ng/ml, 200 ng/ml, 500 ng/ml, or 990 ng/ml. Those of skill in the art will understand that the amounts given here apply to a full-length antibody or immunoglobulin molecule. If an antigen binding fragment thereof is used, the absolute quantity will differ from that given in a manner that can be calculated based on the molecular weight of the fragment relative to the molecular weight of the full-length antibody.

In exemplary embodiments, treatment of a subject with an IL-23 specific antibody is given in an amount and at an interval of 15-54 mg every 0.5-1.5 months; 55-149 mg every 1.5-4.5 months; 150-299 mg every 4-8 months; 300-1100 mg every 8-14 months, or 300-1100 mg every 4-12 months. In some embodiments, the anti-IL-23 antibody is administered at dosages of 15-21 mg every 0.5-1.0 month, 55-70 mg every 1.5-3.0 months, 150-260 mg every 4-6 months, or 300-700 mg every 4-8 months. In some embodiments, the amount and interval are selected from the group consisting of: 21 mg every month; 70 mg every 3 months; 210 mg every 6 months; or 700 mg every 6 months. In some embodiments, an anti-IL-23 antibody is administered at 260 mg every 3 months or at 700 mg every 3 months. In some embodiments, 260 mg of the antibody is administered every 1 month or 700 mg is administered every 1 month.

Administration and dosage regimens of an anti-IL-23 antibody can be adjusted to provide an effective amount for an optimum therapeutic response. For example, a single bolus can be administered, several divided doses can be administered over time or the dose can be proportionally reduced or increased as indicated by the exigencies of the therapeutic situation.

The anti-IL-23 therapies disclosed herein are also contemplated for use with more than one anti-IL-23 therapeutic, or with other therapies. When multiple therapeutics are co-administered, dosages may be adjusted accordingly, as is recognized or known in the pertinent art.

Particular embodiments of methods and compositions of the invention involve the use of an IL-23 antigen binding protein and one or more additional IL-23 antagonists, for example, two or more antigen binding proteins of the invention, or an antigen binding protein of the invention and one or more other IL-23 antagonists. Also provided are IL-23 antigen binding proteins administered alone or in combination with other agents useful for treating the condition with which the patient is afflicted. Examples of such agents include both proteinaceous and non-proteinaceous drugs. Such agents include therapeutic moieties having anti-inflammatory properties (for example, non-steroidal anti-inflammatory agents, steroids, immunomodulators and/or other cytokine inhibitors such as those that antagonize, for example, IFN-γ, GM-CSF, IL-6, IL-8, IL-17, IL-22 and TNFs), or of an IL-23 antigen binding protein and one or more other treatments (e.g., surgery, ultrasound, or treatment effective to reduce inflammation). When multiple therapeutics are co-administered, dosages may be adjusted accordingly, as is recognized or known in the pertinent art. Useful agents that may be combined with IL-23 antigen binding proteins include those used to treat, for example, Crohn's disease or ulcerative colitis, such as aminosalicylate (for example, mesalamine), corticosteroids (including prednisone), antibiotics such as metronidazole or ciprofloxacin (or other antibiotics useful for treating, for example, patients afflicted with fistulas), and immunosuppressives such as azathioprine, 6-mercaptopurine, methotrexate, tacrolimus and cyclosporine. Such agent(s) may be administered orally or by another route, for example via suppository or enema. Agents which may be combined with IL-23 binding proteins in treatment of psoriasis include corticosteroids, calcipotriene and other vitamin D derivatives, acetretin and other retinoic acid derivatives, methotrexate, tacrolimus, and cyclosporine used topically or systemically. Such agents can be administered simultaneously, consecutively, alternately, or according to any other regimen that allows the total course of therapy to be effective.

In addition to human patients, IL-23 antigen binding proteins are useful in the treatment of non-human animals such as domestic pets (dogs, cats, birds, primates, and the like), and domestic farm animals (horses cattle, sheep, pigs, birds, and the like). In such instances, an appropriate dose may be determined according to the animal's body weight. For example, a dose of 0.2-1 mg/kg may be used. Alternatively, the dose is determined according to the animal's surface area, an exemplary dose ranging from 0.1-20 mg/m², or more preferably, from 5-12 mg/m². For small animals, such as dogs or cats, a suitable dose is 0.4 mg/kg. IL-23 antigen binding protein (preferably constructed from genes derived from the recipient species) is administered by injection or other suitable route one or more times per week until the animal's condition is improved, or it may be administered indefinitely.

In one aspect of the invention, the level of IL-22BP in a brazikumab non-responder may be at least 359 pg/mL, e.g., 359 pg/mL to 6,000 pg/mL, where a subject having a serum level of IL-22BP below such level is responsive to an anti-IL-23 agent for an inflammatory condition. The level of IL-22BP in a control may be determined based on subjects not having such inflammatory condition, previously determined to be responsive to brazikumab or previously determined to be non-responsive to brazikumab. In some embodiments, the level of IL-22BP in a brazikumab non-responder is 359-5,000 pg/mL, 359-4,000 pg/mL, 359-2,500 pg/mL, 359-1,000 pg/mL, 359-500 pg/mL, 400-6,000 pg/mL, 400-5,000 pg/mL, 400-4,000 pg/mL, 400-2,500 pg/mL, 400-1,000 pg/mL, 400-500 pg/mL 500-6,000 pg/mL, 500-5,000 pg/mL, 500-4,000 pg/mL, 500-2,500 pg/mL, 500-1,000 pg/mL, 750-6,000 pg/mL, 750-5,000 pg/mL, 750-4,000 pg/mL, 750-2,500 pg/mL, 750-1,000 pg/mL, 1,000-6,000 pg/mL, 1,000-5,000 pg/mL, 1,000-4,000 pg/mL, 1,000-2,500 pg/mL, 1,500-6,000 pg/mL, 1,500-5,000 pg/mL, 1,500-4,000 pg/mL, 1,500-2,500 pg/mL, 2,000-6,000 pg/mL, 2,000-5,000 pg/mL, or 2,000-2,500 pg/mL.

In one aspect of the invention, the level of IFN-γ in a Brazikumab non-responder is less than 15 pg/mL, where a subject having a serum level of IFN-γ of at least 15 pg/mL is responsive to an anti-IL-23 agent for an inflammatory condition. The level of IFN-γ in a control may be determined based on subjects not having such inflammatory condition, previously determined to be responsive to brazikumab or previously determined to be non-responsive to brazikumab.

The following examples, including the experiments conducted and the results achieved, are provided for illustrative purposes only and are not to be construed as limiting the scope of the appended claims.

### EXAMPLES

### Example 1

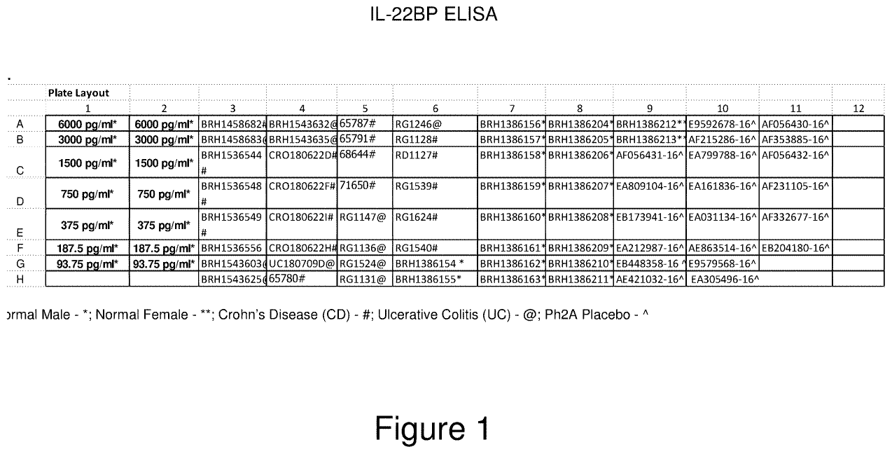

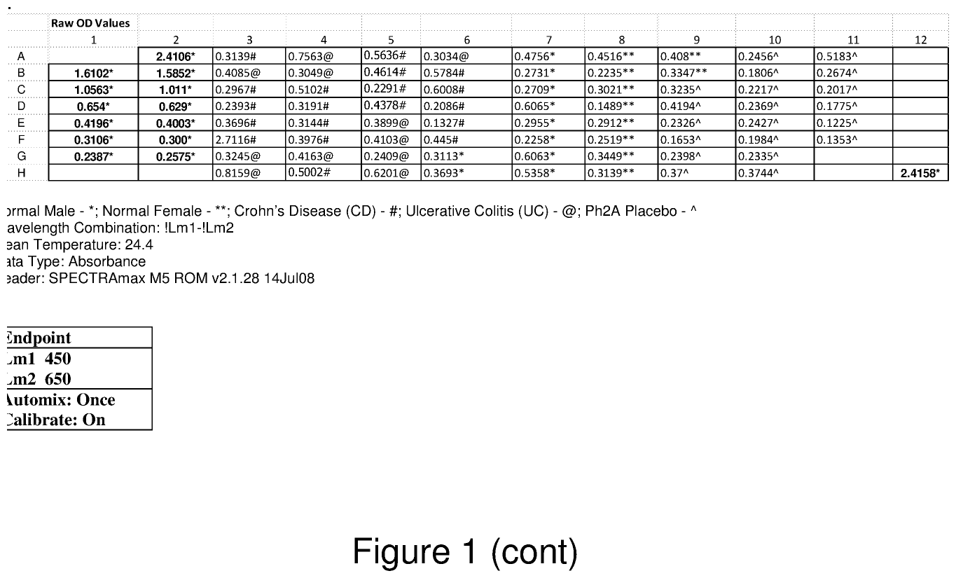

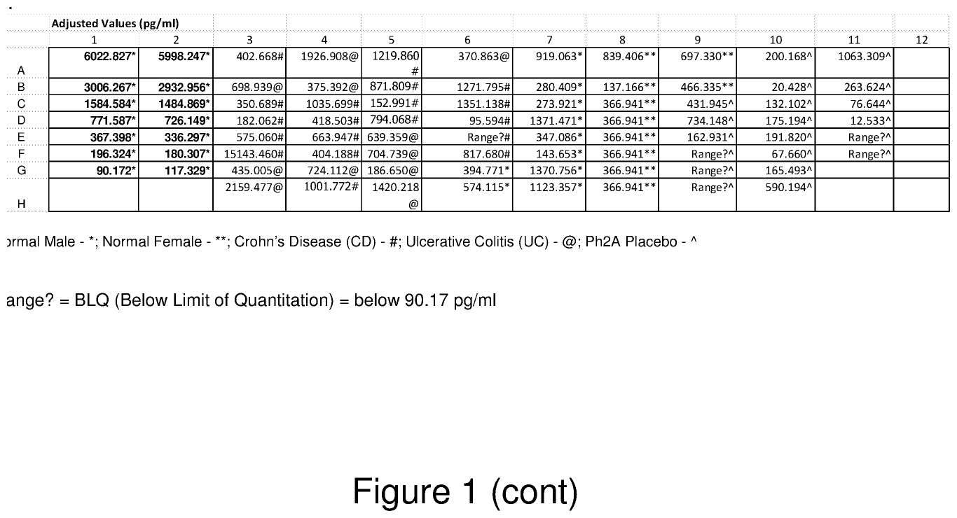

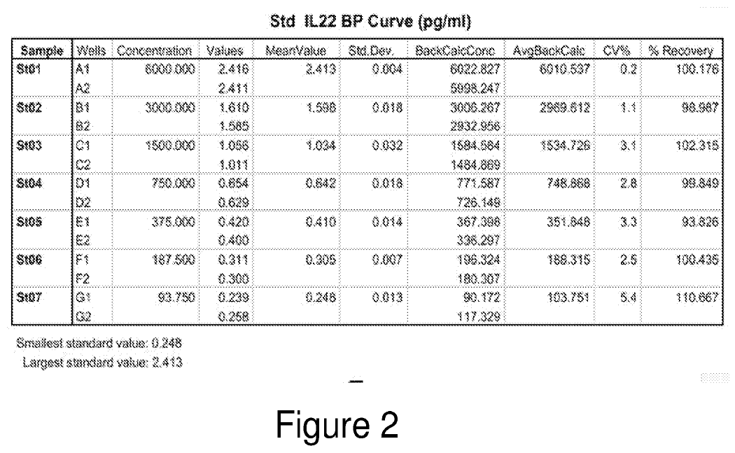

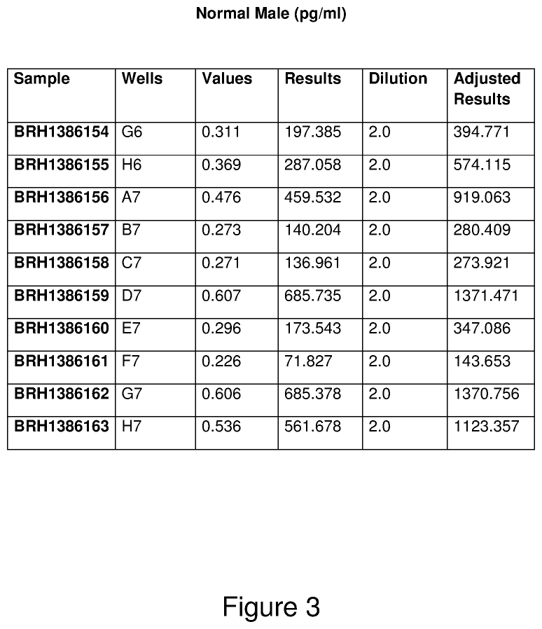

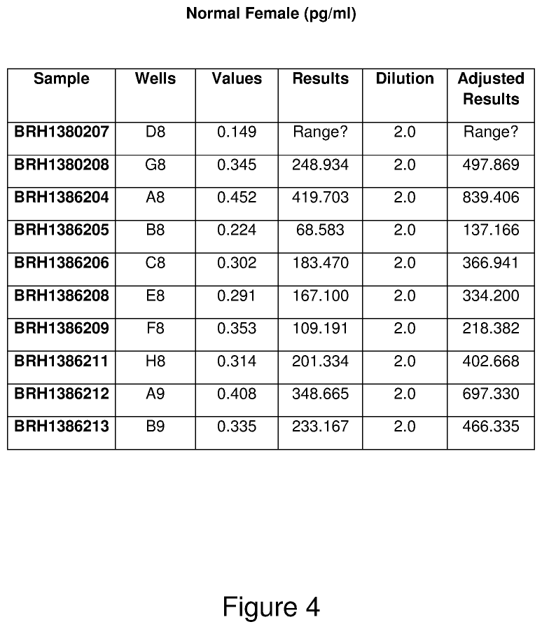

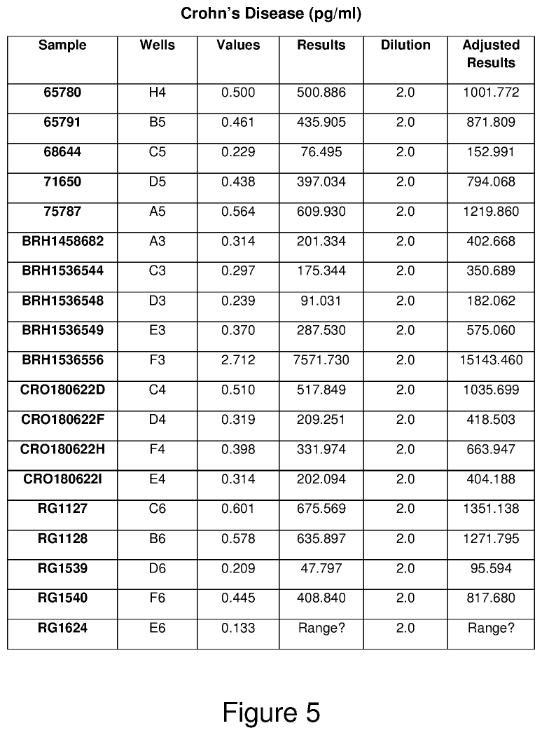

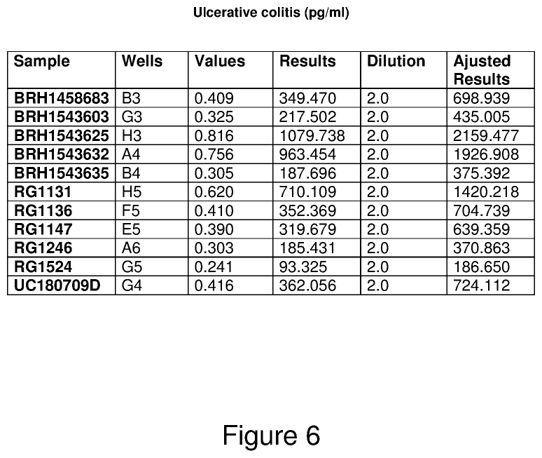

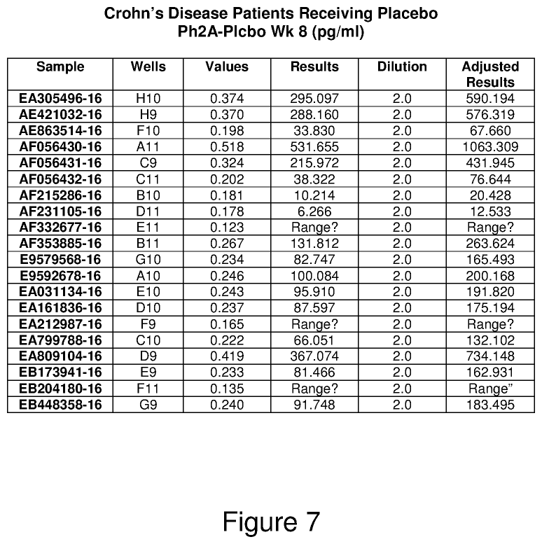

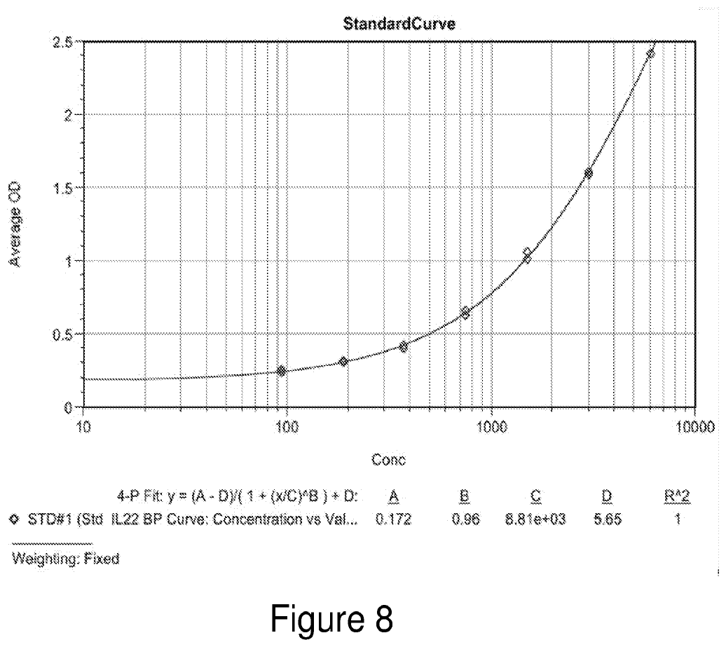

Data generated from experiments described in these examples, and presented in FIGS. 1-10, were obtained by Enzyme-Linked ImmunoSorbent Assays (ELISAs) using R&D System's commercial IL-22BP kit (R&D Systems, Inc., Minneapolis, Minn.). Serum levels of IL-22BP from healthy humans were compared to Crohn's disease (CD) patients, Ulcerative Colitis (UC) patients and Crohn's Disease patients refractory to anti-TNFα treatment. Results showed that the serum level of IL-22BP in CD and UC patients were higher than in healthy humans. The IL-22BP levels in CD patients refractory to anti-TNFα treatment, however, were lower than in healthy humans.

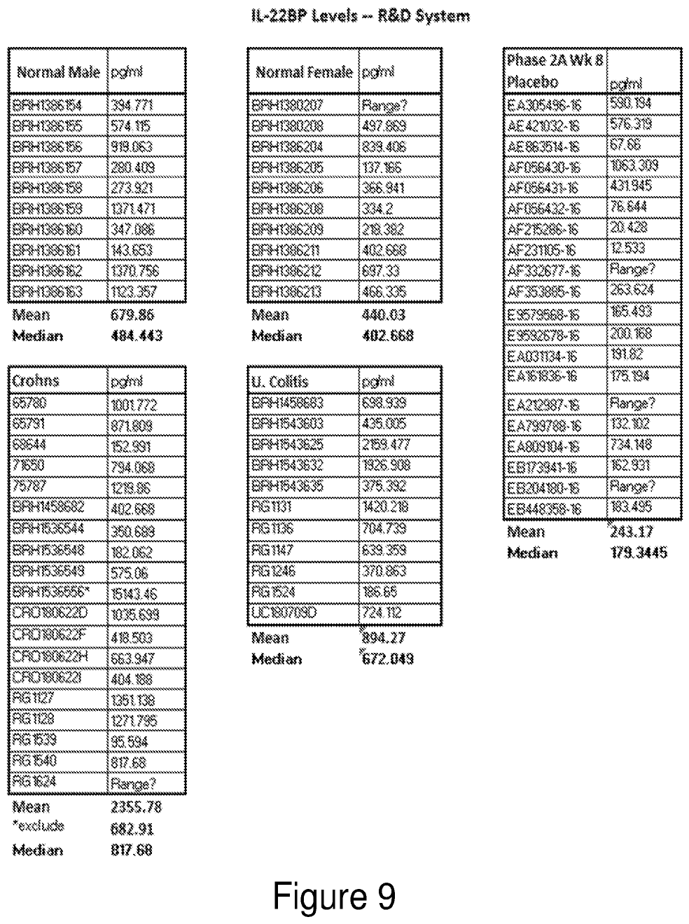

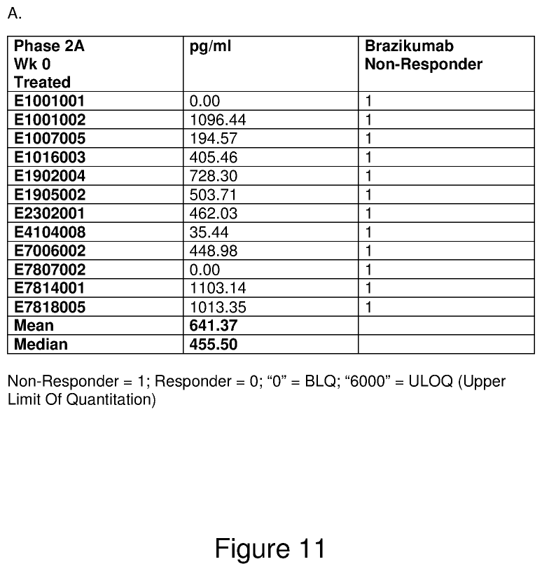

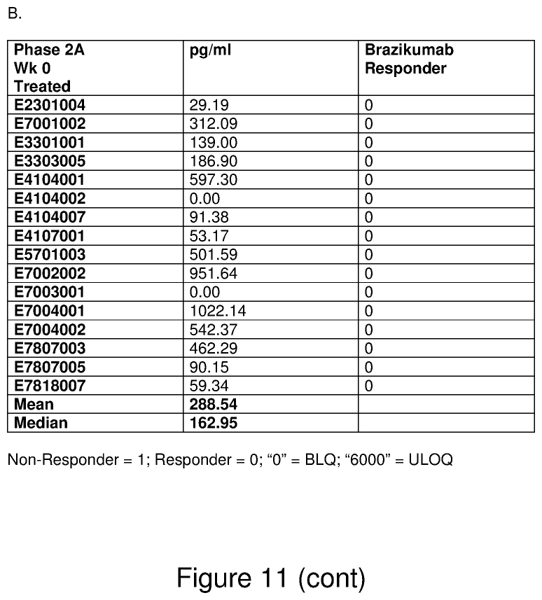

In IBD patients' inflamed intestinal tissues, IL-23 regulated inflammatory cytokines, including IL-22, IL-17a and IFN-γ, are increased compared to healthy controls. Unexpectedly, IL-22BP, a soluble inhibitor of IL-22, was also increased, indicating a tight regulation of IL-22 activity in the gut. Data disclosed herein from a phase 2a clinical trial (i.e., MedI phase 2a clinical trial) indicate that systemic (serum) levels of IL-22BP in IBD patients, which were all non-responders to anti-TNFα treatment, are lower than in healthy controls. See FIG. 9 and FIG. 11. Median serum level of IL-22BP in general CD patients, however, is higher than that of healthy controls. MedI phase 2a results revealed that elevated IL-22 baseline level in TNF-refractory, active CD patients is strongly associated with Brazikumab treatment efficacy. That is to say, a higher baseline level of IL-22 in these patients can predict the patient subpopulation most likely to benefit from Brazikumab treatment. Because a high level of IL-22 is in this case associated with reduced level of IL-22BP in anti-TNFα non-responders, the disclosure provides a method of identifying a patient or patient sub-population amenable to treatment with anti-IL-23 agent due to reduced IL-22BP baseline serum level, which is disclosed herein as a predictive biomarker for patients or patient subgroups more likely to benefit from anti-IL-23 agent treatment than patients not exhibiting reduced IL-22BP baseline serum levels. The subgroups of patients in FIG. 11 are identified from the patient populations that failed anti-TNF agent treatment, i.e., patients that were non-responsive to such treatment, were intolerant to such treatment, or were naïve IBD patients (i.e., patients having received no IBD treatment). The data presented in FIGS. 1-8, as summarized in FIGS. 9 and 11, show that a mean serum level of IL-22BP in normal or healthy human males is 680 pg/ml (679.86 pg/ml) and the mean serum level in normal or healthy human females is 440 pg/ml (440.03 pg/ml). Thus, healthy humans have a mean serum level of IL-22BP no greater than 680 pg/ml. In contrast, humans with Crohn's disease exhibit a mean serum level of IL-22BP of 2350 pg/ml (2355.78 pg/ml). The summarized data in FIG. 9 also establish that humans with Ulcerative Colitis have a mean serum level of IL-22BP of 895 pg/ml (894.27 pg/ml).

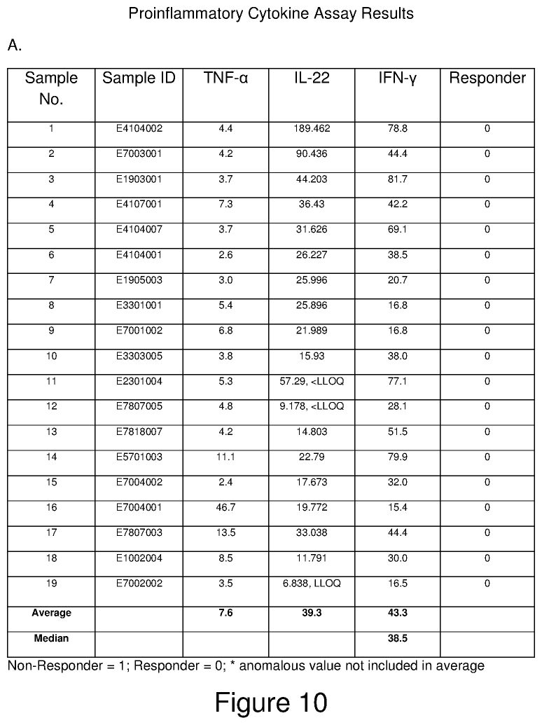

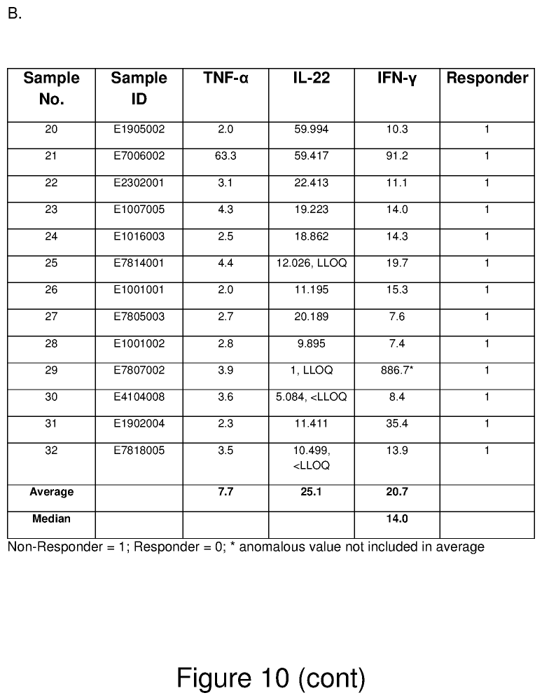

Serum samples from Crohn's disease patients refractory to anti-TNF-α therapy were also subjected to ELISA assays for serum Interferon-γ levels. Immunoassays were performed using the Meso Scale Discovery (NSD) 9plex cytokine assay kit (Meso Scale Discovery, Rockville, Md.). Serum samples were obtained from Crohn's disease patients participating in the Medimmune phase 2a (MED12070-1147) clinical trial. All samples were obtained from patients in the Brazikumab treatment group, and all patients providing samples were non-responders to anti-TNF-α treatment, as noted above. Results shown in FIG. 10 demonstrated that Brazikumab responders have higher serum levels of IFN-γ than non-responders, indicating that the serum level of IFN-γ is useful in identifying the CD patient subpopulation that would be responsive to Brazikumab treatment.

### Example 2

Retrospective analysis of baseline IFN-γ serum levels showed that elevated levels of IFN-γ are associated with a positive response to Brazikumab treatment in patients that had failed, were non-responsive, or were intolerant to treatment with an anti-TNF agent. See FIG. 10. Therefore, elevated baseline IFN-γ serum levels are predictive of, or identify, the TNF-refractory patient subpopulation that will benefit from anti-IL-23 treatment.

All publications and patents mentioned in the application are herein incorporated by reference in their entireties or in relevant part, as would be apparent from context. Various modifications and variations of the disclosed subject matter will be apparent to those skilled in the art without departing from the scope and spirit of the disclosure. Although the disclosure has been described in connection with specific preferred embodiments, it should be understood that the invention as claimed should not be unduly limited to such specific embodiments. Various modifications of the described modes for making or using the disclosed subject matter that are obvious to those skilled in the relevant field(s) are intended to be within the scope of the following claims.

1. A method of selecting a subject with an inflammatory condition amenable to treatment with Brazikumab, comprising:
(a) measuring the serum level of Interleukin-22 Binding Protein (IL-22BP) in a subject;
(b) comparing the serum level of IL-22BP in the subject to a serum level of IL-22BP in a control, wherein the control is one or more individuals without an inflammatory condition; and
(c) selecting the subject as having an inflammatory condition amenable to treatment with Brazikumab if the serum level of IL-22BP is lower in the subject than in the control.

2. (canceled)

3. The method of claim 1 wherein the inflammatory condition is an inflammatory bowel disease, psoriasis, psoriatic arthritis, rheumatoid arthritis, or ankylosing spondylitis.

4. The method of claim 3 wherein the inflammatory bowel disease is Crohn's disease or ulcerative colitis.

5. The method of claim 1 wherein the inflammatory condition is refractory to Tumor Necrosis Factor (TNF) treatment.

6. The method of claim 1 wherein the serum level of Interleukin-22 Binding Protein is less than 359 pg/mL.

7. (canceled)

8. The method of claim 1 further comprising administering Brazikumab in an amount effective to treat the inflammatory condition.

9. (canceled)

10. The method of claim 8 wherein the Brazikumab is administered in an amount sufficient to achieve a serum concentration of from 12.5 ng/ml to 1,000 ng/ml.

11-12. (canceled)

13. A method of treating an interleukin-23 (IL-23)-mediated inflammatory condition in a patient comprising administering an effective amount of Brazikumab to a patient if the patient is determined to have a serum level of IL-22BP that is lower than an IL-22BP level in a control sample, wherein the control sample is obtained from one or more individuals without an inflammatory condition.

14. (canceled)

15. The method of claim 13 wherein the inflammatory condition is an inflammatory bowel disease.

16. The method of claim 15 wherein the inflammatory bowel disease is Crohn's disease or ulcerative colitis.

17. The method of claim 13 wherein the inflammatory condition is refractory to Tumor Necrosis Factor (TNF) treatment.

18. The method of claim 13 wherein the Brazikumab is administered in an amount sufficient to achieve a serum concentration of from 12.5 ng/ml to 1,000 ng/ml.

19-20. (canceled)

21. The method of claim 3 wherein the inflammatory bowel disease (IBD) patient has IBD refractory to Tumor Necrosis Factor (TNF) treatment, has IBD naïve to treatment therefor, and/or the patient is intolerant to treatment with an anti-TNF agent, comprising:
(a) measuring the serum level of Interleukin-22 Binding Protein (IL-22BP) in the IBD patient;
(b) comparing the serum IL-22BP level in the IBD patient to a serum level of IL-22BP in a control, wherein the serum level of IL-22BP in a control is any one of the serum level of IL-22BP in an individual without IBD, the mean level of IL-22BP in a plurality of individuals without an IBD, or the mean value of IL-22BP in a plurality of individuals with an IBD; and
(c) selecting the patient as having an IBD amenable to treatment with Brazikumab if the serum level of IL-22BP is lower in the subject than in the control.

22. (canceled)

23. The method of claim 21 wherein the IBD patient is a Crohn's disease patient or an ulcerative colitis patient.

24-27. (canceled)

28. The method of claim 21 further comprising administering Brazikumab in an amount effective to treat the inflammatory bowel condition.

29. (canceled)

30. The method of claim 28 wherein the Brazikumab is administered in an amount sufficient to achieve a serum concentration of from 12.5 ng/ml to 1,000 ng/ml.

31-38. (canceled)

39. A method of selecting a subject with an inflammatory condition amenable to treatment with Brazikumab from a patient population having an inflammatory condition refractory to Tumor Necrosis Factor treatment, comprising:
(a) measuring the serum level of Interleukin-22 Binding Protein (IL-22BP) in a subject;
(b) comparing the serum IL-22BP level in the subject to a serum level of IL-22BP in a control, wherein the control is one or more individuals without an inflammatory condition refractory to Tumor Necrosis Factor treatment; and
(c) selecting the subject as having an inflammatory condition amenable to treatment with Brazikumab if the serum level of IL-22BP is lower in the subject than in the control.

40-57. (canceled)

58. A method of selecting a subject with an inflammatory condition amenable to treatment with Brazikumab, comprising:
(a) measuring the serum level of Interferon-γ (IFN-γ) in a subject;
(b) comparing the serum level of IFN-γ in the subject to a serum level of IFN-γ in a control, wherein the control is one or more individuals without an inflammatory condition; and
(c) selecting the subject as having an inflammatory condition amenable to treatment with Brazikumab if the serum level of IFN-γ is higher in the subject than in the control.

59-60. (canceled)

61. The method of claim 58 wherein the inflammatory condition is Crohn's disease or ulcerative colitis.

62. The method of claim 58 wherein the selected subject has a serum concentration of Interferon-γ that is greater than 15 pg/mL.

63. The method of claim 58 wherein the inflammatory condition is refractory to Tumor Necrosis Factor treatment.

64. (canceled)

65. The method of claim 58 further comprising administering the Brazikumab in an amount effective to treat the inflammatory condition.

66. (canceled)

67. The method of claim 65 wherein the anti-IL-23 agent is administered in an amount sufficient to achieve a serum concentration of from 12.5 ng/ml to 1,000 ng/ml.

68-78. (canceled)

79. The method of claim 65 wherein the inflammatory bowel disease (IBD) patient has IBD refractory to Tumor Necrosis Factor (TNF) treatment, has IBD naïve to treatment therefor, and/or the patient is intolerant to treatment with an anti-TNF agent, comprising:
(a) measuring the serum level of Interferon-γ (IFN-γ) in the IBD patient;
(b) comparing the serum IFN-γ level in the IBD patient to a serum level of IFN-γ in a control, wherein the serum level of IFN-γ in a control is any one of the serum level of IFN-γ in an individual without IBD, the mean level of IFN-γ in a plurality of individuals without an IBD, or the mean value of IFN-γ in a plurality of individuals with an IBD; and
(c) selecting the patient as having an IBD amenable to treatment with Brazikumab if the serum level of IFN-γ is higher in the subject than in the control.

80. (canceled)

81. The method of claim 79 wherein the IBD patient is a Crohn's disease patient or an ulcerative colitis patient.

82-84. (canceled)

85. The method of claim 79 wherein the selected subject has a serum concentration of Interferon-γ that is greater than 15 pg/mL.

86-125. (canceled)

126. The method of claim 65 further comprising administering an effective amount of Brazikumab to the patient if the serum level of IL-22BP is lower in the patient than in the control.

127-129. (canceled)


SEQUENCE LISTING (154 sequences)

SEQ ID NO: 1 - protein, 111 aa, Homo sapiens
QSVLTQPPSVSGAPGQRVTISCTGSSSNTGAGYDVHWYQQVPGTAPKLLIYGSGNRPSGVPDRFSGSKSGTSASLAITGLQAEDEADYYCQSYDSSLSGWVFGGGTRLTVL

SEQ ID NO: 2 - DNA, 333 bp, Homo sapiens
cagtctgtgctgacgcagccgccctcagtgtctggggccccagggcagagggtcaccatctcctgcactgggagcagctccaacaccggggcaggttatgatgtacactggtaccagcaagttccaggaacagcccccaaactcctcatttatggtagcggcaatcggccctcaggggtccctgaccgattctctggctccaagtctggcacctcagcctccctggccatcactggactccaggctgaggatgaggctgattattactgccagtcctatgacagcagcctgagtggttgggtgttcggcggagggaccaggctgaccgtcctg

SEQ ID NO: 3 - protein, 111 aa, Homo sapiens
QSVLTQPPSVSGAPGQRVTISCTGSSSNIGAGYDVHWYQQLPGTAPKLLIYGSNNRPSGVPDRFSGSKSGTSASLAITGLQAEDEADYYCQSYDSSLSGWVFGGGTKLTVL

SEQ ID NO: 4 - protein, 115 aa, Homo sapiens
QAVLTQPSSLSASPGASASLTCTLRSGINVGTYRIYWYQQKPGSPPQYLLRYKSDSDKQQGSGVPSRFSGSKDASANAGILLISGLQSEDEADYYCMIWHSSASVFGGGTKLTVL

SEQ ID NO: 5 - DNA, 345 bp, Homo sapiens
caggctgtgctgactcagccgtcttccctctctgcatctcctggagcatcagccagtctcacctgcaccttacgcagtggcatcaatgttggtacctacaggatatactggtaccagcagaagccagggagtcctccccagtatctcctgaggtacaaatcagactcagataagcagcagggctctggagtccccagccgcttctctggatccaaagatgcttcggccaatgcagggattttactcatctctgggctccagtctgaggatgaggctgactattactgtatgatttggcacagcagcgcttcggtattcggcggagggaccaagctgaccgtccta

SEQ ID NO: 6 - protein, 14 aa, Homo sapiens
ENTVTIYYNYGMDV

SEQ ID NO: 7 - protein, 116 aa, Homo sapiens
QPVLTQPPSASASLGASVTLTCTLNSGYSDYKVDWYQQRPGKGPRFVMRVGTGGIVGSKGDGIPDRFSVLGSGLNRYLTIKNIQEEDESDYHCGADHGSGSNFVYVFGTGTKVTVL

SEQ ID NO: 8 - DNA, 348 bp, Homo sapiens
cagcctgtgctgactcagccaccttctgcatcagcctccctgggagcctcggtcacactcacctgcaccctgaacagcggctacagtgattataaagtggactggtaccagcagagaccagggaagggcccccggtttgtgatgcgagtgggcactggtgggattgtgggatccaagggggatggcatccctgatcgcttctcagtcttgggctcaggcctgaatcggtacctgaccatcaagaatatccaggaagaggatgagagtgactaccactgtggggcagaccatggcagtgggagcaacttcgtgtatgtcttcggaactgggaccaaggtcaccgtccta

SEQ ID NO: 9 - protein, 116 aa, Homo sapiens
QPVLTQPPSASASLGASVTLTCTLSSGYSDYKVDWYQQRPGKGPRFVMRVGTGGIVGSKGEGIPDRFSVLGSGLNRYLTIKNIQEEDESDYHCGADHGSGNNFVYVFGTGTKVTVL

SEQ ID NO: 10 - DNA, 348 bp, Homo sapiens
cagcctgtgctgactcagccaccttctgcatcagcctccctgggagcctcggtcacactcacctgcaccctgagcagcggctacagtgattataaagtggactggtaccagcagagaccagggaagggcccccggtttgtgatgcgagtgggcactggtgggattgtgggatccaagggggaaggcatccctgatcgcttctcagtcttgggctcaggcctgaatcggtacctgaccatcaagaacatccaggaagaggatgagagtgactaccactgtggggcagaccatggcagtgggaacaacttcgtgtatgtcttcggaactgggaccaaggtcaccgtccta

SEQ ID NO: 11 - protein, 116 aa, Homo sapiens
QPELTQPPSASASLGASVTLTCTLSSGYSDYKVDWYQLRPGKGPRFVMRVGTGGTVGSKGEGIPDRFSVLGSGLNRSLTIKNIQEEDESDYHCGADHGSGSNFVYVFGTGTKVTVL

SEQ ID NO: 12 - DNA, 348 bp, Homo sapiens
cagcctgagttgactcagccaccttctgcatcagcctccctgggagcctcggtcacactcacctgcaccctgagcagcggctacagtgattataaagtggactggtaccagctgagaccagggaagggcccccggtttgtgatgcgagtgggcactggtgggactgttggatccaagggggaaggcatccctgatcgcttctcagtcttgggctcaggcctgaatcggtccctgaccatcaagaacatccaggaagaggatgagagtgactaccactgtggggcagaccatggcagtgggagcaacttcgtgtatgtcttcggaactgggaccaaggtcaccgtccta

SEQ ID NO: 13 - protein, 107 aa, Homo sapiens
DIQLTPSPSSVSASVGDRVTITCRASQGIAGWLAWYQQKPGKAPKLLIYAASSLQSGVPSRFSGSGSGTDFTLTISSLQPEDFATYYCQQADSFPPTFGGGTKVEIK

SEQ ID NO: 14 - DNA, 321 bp, Homo sapiens
gacatccagttgaccccgtctccatcttccgtgtctgcatctgtaggagacagagtcaccatcacttgtcgggcgagtcagggtattgccggctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctgacagtttccctcccactttcggcggagggaccaaggtggagatcaaa

SEQ ID NO: 15 - protein, 107 aa, Homo sapiens
DIQMTQSPSSVSASVGDRVTITCRASQVISSWLAWYQQKPGKAPSLLIYAASSLQSGVPSRFSGSVSGTDFTLTISSLQPEDFATYYCQQANSFPFTFGPGTKVDFK

SEQ ID NO: 16 - DNA, 321 bp, Homo sapiens
gacatccagatgacccagtctccatcttccgtgtctgcatctgtaggagacagagtcaccatcacttgtcgggcgagtcaggttattagcagctggttagcctggtatcagcagaaaccagggaaagcccctagcctcctgatctatgctgcatccagtttgcaaagtggggtcccatcaaggttcagcggcagtgtatctgggacagatttcactctcaccatcagcagcctgcagcctgaagattttgcaacttactattgtcaacaggctaacagtttcccattcactttcggccctgggaccaaagtggatttcaaa

SEQ ID NO: 17 - protein, 107 aa, Homo sapiens
DIQMTQSPSSVSASVGDRVTITCRASQGSSSWFAWYQQKPGKAPKLLIYAASSLQSGVPSRFSGSGSGTDFTLTISSLQPEDFATYYCQQANSFPFTFGPGTKVDIK

SEQ ID NO: 18 - DNA, 321 bp, Homo sapiens
gacatccagatgacccagtctccatcttccgtgtctgcatctgtaggagacagagtcaccatcacttgtcgggcgagtcagggaagtagcagctggtttgcctggtatcagcagaaaccagggaaagccccaaagctcctgatctatgctgcatccagtttgcaaagtggggtcccatcaaggttcagcggcagtggatctgggacagacttcactctcaccatcagcagcctgcagcctgaagattttgcaacttactattgtcaacaggctaacagtttcccattcactttcggccctgggaccaaagtggatatcaaa

SEQ ID NO: 19 - protein, 107 aa, Homo sapiens
DSQMTQSPSSVSASVGDRVTITCRASQGISSWFAWYQQKPGQAPNLLIYAASSLQSGVPSRFSGSGSGTEFTLTISSLQPEDFATYYCQQANSFPFTFGPGTKVDIK

SEQ ID NO: 20 - DNA, 321 bp, Homo sapiens
gacagccagatgacccagtctccatcttccgtgtctgcctctgtaggagacagagtcaccatcacttgtcgggcgagtcagggtattagcagctggtttgcctggtatcagcagaaaccagggcaagcccctaacctcctgatctatgctgcatccagtttgcaaagtggggtcccatcaaggttcagcggcagtggatctgggacagaattcactctcaccatcagcagcctgcagcctgaagattttgcaacttactattgtcaacaggctaacagtttcccattcactttcggccctgggaccaaagtggatatcaaa

SEQ ID NO: 21 - protein, 107 aa, Homo sapiens
DIQMTQSPSSVSASVGDRVTITCRAGQVISSWLAWYQQKPGKAPKLLIYAASSLQSGVPSRFSGSGSGTDFTLTISSLQPDDFATYYCQQATSFPLTFGGGTKVEIK

SEQ ID NO: 22 - DNA, 321 bp, Homo sapiens
gacatccagatgacccagtctccatcttccgtgtctgcatctgtaggagacagagtcaccatcacttgtcgggcgggtcaggttattagcagctggttagcctggtatcagcagaaaccagggaaagcccctaagctcctgatctatgctgcatccagtttgcaaagtggggtcccatcgaggttcagcggcagtggatctgggacagatttcactctcaccatcagcagcctgcagcctgacgattttgcaacttactattgtcaacaggctaccagttttcccctcactttcggcggagggaccaaggtggagatcaaa

SEQ ID NO: 23 - protein, 107 aa, Homo sapiens
DIQMTQSPSSVSASVGDRVTITCRASQGFSGWLAWYQQKPGKAPKLLIYAASSLQSGVPSRFSGSGSGTDFTLTISSLQPEDFATYYCQQANSFPFTFGPGTKVDIK

SEQ ID NO: 24 - DNA, 321 bp, Homo sapiens
gacatccagatgacccagtctccatcttccgtgtctgcatctgtaggagacagagtcaccatcacttgtcgggcgagtcagggttttagcggttggttagcctggtatcagcagaaaccagggaaagcccctaagctcctgatctatgctgcatccagtttgcaaagtggggtcccatcaaggttcagtggcagtggatctgggacagatttcactctcaccatcagcagcctgcagcctgaagattttgcaacttactactgtcaacaggctaacagtttcccattcactttcggccctgggaccaaagtggatatcaaa

SEQ ID NO: 25 - protein, 107 aa, Homo sapiens
DIQLTQSPSSVSASVGDRVTITCRASQVISSWFAWYQQKPGKAPNLLIYAASSLQSGVPSRFSGSGSGTDFTLTISSLQPADFATYFCQQANSFPFTFGPGTKVDVK

SEQ ID NO: 26 - DNA, 321 bp, Homo sapiens
gacatccagttgacccagtctccatcttccgtgtctgcatctgtaggagacagagtcaccatcacttgtcgggcgagtcaggttattagcagctggtttgcctggtatcagcagaaaccagggaaagcccctaacctcctgatctatgctgcatccagtttgcaaagtggggtcccatcaaggttcagcggcagtggatctgggacagatttcactctcaccatcagcagcctgcagcctgcagattttgcaacttacttttgtcaacaggctaacagtttcccattcactttcggccctgggaccaaagtggatgtcaaa

SEQ ID NO: 27 - protein, 107 aa, Homo sapiens; identical to SEQ ID NO: 17

SEQ ID NO: 28 - DNA, 321 bp, Homo sapiens
gacatccagatgacccagtctccatcttccgtgtctgcatctgtaggagacagagtcaccatcacttgtcgggcgagtcagggtagtagcagctggtttgcctggtatcaacagaaaccagggaaagccccaaagctcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttcccattcactttcggccctgggaccaaagtggatatcaaa

SEQ ID NO: 29 - protein, 107 aa, Homo sapiens
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPPTFGQGTKVEIE

SEQ ID NO: 30 - protein, 108 aa, Artificial
  Consensus Sequence
  MISC_FEATURE (2)..(2): Xaa can be Ile or Ser
  MISC_FEATURE (4)..(4): Xaa can be Met or Leu
  MISC_FEATURE (29)..(29): Xaa can be Gly or Val
  MISC_FEATURE (30)..(30): Xaa can be Ser, Phe or Ile
  MISC_FEATURE (32)..(32): Xaa can be Ser or Gly
  MISC_FEATURE (34)..(34): Xaa can be Phe or Leu
  MISC_FEATURE (43)..(43): Xaa can be Lys or Gln
  MISC_FEATURE (46)..(46): Xaa can be Lys, Asn or Ser
  MISC_FEATURE (67)..(67): Xaa can be Gly or Val
  MISC_FEATURE (71)..(71): Xaa can be Asp or Glu
  MISC_FEATURE (82)..(82): Xaa can be Glu or Ala
  MISC_FEATURE (88)..(88): Xaa can be Tyr or Phe
  MISC_FEATURE (107)..(107): Xaa can be Ile, Val or Phe
DXQXTQSPSSVSASVGDRVTITCRASQGXXSXWXAWYQQKPGXAPXLLIYAASSLQSGVPSRFSGSXSGTXFTLTISSLQPXDFATYXCQQANSFPFTFGPGTKVDXK

SEQ ID NO: 31 - protein, 124 aa, Homo sapiens
QVQLVESGGGVVQPGRSLRLSCAASGFTFSSYGMHWVRQAPGKGLEWVAVIWYDGSNEYYADSVKGRFTISRDNSKNTLYLQMNSLRAEDTAVYYCARDRGYTSSWYPDAFDIWGQGTMVTVSS

SEQ ID NO: 32 - DNA, 372 bp, Homo sapiens
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaatgaatactatgcagactccgtgaagggccgattcaccatctccagagacaattccaagaacacgctgtatctgcaaatgaacagcctgagagccgaggacacggctgtgtattactgtgcgagagatcgggggtataccagtagctggtaccctgatgcttttgatatctggggccaagggacaatggtcaccgtctcttca

SEQ ID NO: 33 - protein, 124 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDRGYSSSWYPDAFDIWGQGTMVTVSS

SEQ ID NO: 34 - protein, 121 aa, Homo sapiens
QVQLVESGGGVVQPGRSLRLSCAASGFTFSSYGMHWVRQAPGKGLEWVAVISFDGSLKYYADSVKGRFTISRDNSKNTLYLQMNSLRAEDTAVYYCARERTTLSGSYFDYWGQGTLVTVSS

SEQ ID NO: 35 - DNA, 363 bp, Homo sapiens
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatttgatggaagtcttaaatactatgcagactccgtgaagggccgattcaccatctccagagacaattccaagaacaccctgtatctgcaaatgaacagcctgagagctgaggacacggctgtgtattactgtgcgagagaacggactactttaagtgggagctactttgactactggggccagggaaccctggtcaccgtctcctca

SEQ ID NO: 36 - protein, 121 aa, Homo sapiens
QVQLVESGGGVVQPGRSLRLSCAASGFTFSSYAMHWVRQAPGKGLEWLSVISHDGSIKYYADSVKGRFTISRDNSKNTLYLQMNSLRAEDTAVYYCARERTTLSGSYFDYWGQGTLVTVSS

SEQ ID NO: 37 - DNA, 363 bp, Homo sapiens
caggtgcagctggtggagtctgggggaggcgtggtccagcctgggaggtccctgagactctcctgtgcagcctctggattcaccttcagtagctatgccatgcactgggtccgccaggctccaggcaaggggctggagtggttgtcagttatatcacatgatggaagtattaaatactatgcagactccgtgaagggccgattcaccatctccagagacaattccaagaacacgctgtatctgcaaatgaacagcctgagagctgaggacacggctgtgtattactgtgcgagagaacggactactctaagtgggagctactttgactactggggccagggaaccctggtcaccgtctcctca

SEQ ID NO: 38 - protein, 125 aa, Homo sapiens
EVQLVESGGGLVQPGGSLRLSCAASGFTFSSYSMNWVRQAPGKGLEWVSYISSRSSTIYIADSVKGRFTISRDNAKNSLYLQMNSLRDEDTAVYYCARRIAAAGGFHYYYALDVWGQGTTVTVSS

SEQ ID NO: 39 - DNA, 375 bp, Homo sapiens
gaggtgcagctggtggagtctgggggaggcctggtacagcctggggggtccctgagactctcctgtgcagcctctggattcaccttcagtagctatagtatgaactgggtccgccaggctccagggaaggggctggagtgggtttcgtacattagtagtaggagtagtaccatatacatcgcagactctgtgaagggccgattcaccatctccagagacaatgccaagaactcactgtatctgcaaatgaacagcctgagagacgaagacacggctgtgtattactgtgcgagacggatagcagcagctggtgggttccactactactacgctttggacgtctggggccaagggaccacggtcaccgtctcctca

SEQ ID NO: 40 - protein, 125 aa, Homo sapiens
EVQLVESGGGLVQPGGSLRLSCAASGFTFSTYSMNWVRQAPGKGLEWVSYISSSSSTRYHADSVKGRFTISRDNAKNSLYLQMNSLRDEDTAVYYCARRIAAAGPWGYYYAMDVWGQGTTVTVSS

SEQ ID NO: 41 - DNA, 375 bp, Homo sapiens
gaggtgcagctggtggagtctgggggaggcttggtacaacctggggggtccctgagactctcctgtgcagcctctggattcaccttcagtacctatagcatgaactgggtccgccaggctccagggaaggggctggagtgggtttcatacattagtagcagtagtagtaccagataccacgcagactctgtgaagggccgattcaccatctccagagacaatgccaagaactcactgtatctgcaaatgaacagcctgagagacgaggacacggctgtgtattactgtgcgagacgtatagcagcagctggtccgtggggctactactacgctatggacgtctggggccaagggaccacggtcaccgtctcctca

SEQ ID NO: 42 - protein, 125 aa, Homo sapiens
EVQLVESGGGLVQPGGSLRLSCVVSGFTFSSFSMNWVRQAPGKGLEWVSYISSRSSTIYYADSVKGRFTISRDNAKNSLYLQMNSLRDEDTAVYYCARRIAAAGPWGYYYAMDVWGQGTTVTVSS

SEQ ID NO: 43 - DNA, 375 bp, Homo sapiens
gaggtgcagctggtggagtctgggggaggcttggtacagcctggggggtccctgagactctcctgtgtagtctctggattcaccttcagtagttttagcatgaactgggtccgccaggctccagggaaggggctggagtgggtttcatacattagtagtcgtagtagtaccatatactacgcagactctgtgaagggccgattcaccatctccagagacaatgccaagaactcactgtatctgcaaatgaacagcctgagagacgaggacacggctgtgtattattgtgcgagacgtatagcagcagctggtccgtggggctactactacgctatggacgtctggggccaagggaccacggtcaccgtctcctca

SEQ ID NO: 44 - protein, 118 aa, Homo sapiens
QVQLQESGPGLVKPSETLSLTCTVSGGSISTYYWSWIRQPAGKGLEWIGLIYTSGSTNYNPSLKSRVTMSLDTSKNQFSLRLTSVTAADTAVYYCARDRGYYYGVDVWGQGTTVTVSS

SEQ ID NO: 45 - DNA, 354 bp, Homo sapiens
caggtgcagctgcaggagtcgggcccaggactggtgaagccttcggagaccctgtccctcacctgcactgtctctggtggctccatcagtacttactactggagctggatccggcagcccgccgggaagggactggagtggattgggcttatctataccagtgggagcaccaactacaacccctccctcaagagtcgagtcaccatgtcattagacacgtccaagaaccagttctccctgaggctgacctctgtgaccgccgcggacacggccgtttattactgtgcgagagatcgtgggtactactacggtgtggacgtctggggccaggggaccacggtcaccgtctcctca

SEQ ID NO: 46 - protein, 120 aa, Homo sapiens
QVQLQESGPGLVKPSQTLSLTCTVSGGSISSGGYYWSWIRQHPGKGLEWIGHIHYSGNTYYNPSLKSRVTISVDTSKNQFSLKLSSVTAADTAVYYCAKNRGFYYGMDVWGQGTTVTVSS

SEQ ID NO: 47 - DNA, 360 bp, Homo sapiens
caggtgcagctgcaggagtcgggcccaggactggtgaagccttcacagaccctgtccctcacctgcactgtctctggtggctccatcagcagtggtggttactactggagctggatccgccagcacccagggaagggcctggagtggattgggcacatccattacagtgggaacacctactacaacccgtccctcaagagtcgagttaccatatcagtagacacgtctaagaatcagttctccctgaaactgagctctgtgactgccgcggacacggccgtgtattactgtgcgaaaaatcgcgggttctactacggtatggacgtctggggccaagggaccacggtcaccgtctcctca

SEQ ID NO: 48 - protein, 120 aa, Homo sapiens
QVQLQESGPGLVKPSQTLSLTCTVSGGSINSGGYYWSWIRQHPGKGLEWIGYIYYSGSSYYNPSLKSRVTISVDTSQNQFSLKLSSVTAADTAVYYCARDRGHYYGMDVWGQGTTVTVSS

SEQ ID NO: 49 - DNA, 360 bp, Homo sapiens
caggtgcagctgcaggagtcgggcccaggactggtgaagccttcacagaccctgtccctcacctgcactgtctctggtggctccatcaacagtggtggttactactggagctggatccgccagcacccagggaagggcctggagtggattgggtacatctattacagtgggagctcctactacaacccgtccctcaagagtcgagttaccatatcagtagacacgtctcagaaccagttctccctgaagctgagctctgtgactgccgcggacacggccgtgtattactgtgcgagagatcgggggcactactacggtatggacgtctggggccaagggaccacggtcaccgtctcctca

SEQ ID NO: 50 - protein, 120 aa, Homo sapiens
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARDRGHYYGMDVWGQGTTVTVSS

SEQ ID NO: 51 - DNA, 360 bp, Homo sapiens
caggtgcagctgcaggagtcgggcccaggactggtgaagccttcacagaccctgtccctcacctgcactgtctctggtggctccatcagtagtggtggttactactggagctggatccgccagcacccagggaagggcctggagtggattgggtacatttattacagtgggagcacctactacaacccgtccctcaagagtcgagttaccatatcagtagacacgtctaagaaccagttctccctgaagctgagctctgtgactgccgcggacacggccgtgtattactgtgcgagagatcggggccactactatggaatggacgtctggggccaagggaccacggtcaccgtctcctca

SEQ ID NO: 52 - protein, 118 aa, Homo sapiens
QVQLQESGPRLVKPSETLSLTCTVSGDSISSYFWSWIRQPPGKGLEWLGYIYYSGSTNYNPSLKSRVTISIDTSKNQFSLKLSSVTAADTAVYYCTRDRGSYYGSDYWGQGTLVTVSS

SEQ ID NO: 53 - DNA, 354 bp, Homo sapiens
caggtgcagctgcaggagtcgggcccaagactggtgaagccttcggagaccctgtccctcacctgcactgtctctggtgactccatcagtagttacttctggagctggatccggcagcccccagggaagggactggagtggcttgggtatatctattacagtgggagcaccaactacaacccctccctcaagagtcgagtcaccatatcaatagacacgtccaagaaccagttctccctgaagctgagctctgtgaccgctgcggacacggccgtgtattactgtacgagagatcgggggagctactacggatctgactactggggccagggaaccctggtcaccgtctcctca

SEQ ID NO: 54 - protein, 120 aa, Homo sapiens
QVQLQESGPGLVKPSQTLSLTCTVSGGSISSGGYYWTWIRQHPGKGLEWIGYIYYSGNTYYNPSLKSRITISVDTSKNQFSLSLSSVTAADTAVYYCARNRGYYYGMDVWGQGTTVTVSS

SEQ ID NO: 55 - DNA, 360 bp, Homo sapiens
caggtgcagctgcaggagtcgggcccaggactggtgaagccttcacagaccctgtccctcacctgcactgtctctggtggctccatcagcagtggtggttactactggacctggatccgccagcacccagggaagggcctggagtggattgggtacatctattacagtgggaacacctactacaacccgtccctcaagagtcgaattaccatatcagtggacacgtctaagaaccagttctccctgagcctgagctctgtgactgccgcggacacggccgtgtattactgtgcgagaaatcgcgggtactactacggtatggacgtctggggccaagggaccacggtcaccgtctcctca

SEQ ID NO: 56 - protein, 120 aa, Homo sapiens
QVQLQESGPGLVKPSQTLSLTCTVSGGSISSGGYYWSWIRQHPGKGLEWIGYIYYSGSTYYNPSLKSRVTMSVDTSKNQFSLKLSSVTAADTAVYYCAKNRGFYYGMDVWGQGTTVTVSS

SEQ ID NO: 57 - DNA, 360 bp, Homo sapiens
caggtgcagctgcaggagtcgggcccaggactggtgaagccttcacagaccctgtccctcacctgcactgtctctggtggctccatcagcagtggtggttactactggagctggatccgccagcacccagggaagggcctggagtggattgggtacatctattacagtgggagcacctactacaacccgtccctcaagagtcgagttaccatgtcagtagacacgtctaagaaccagttctccctgaaactgagctctgtgactgccgcggacacggccgtgtattactgtgcgaaaaatcgcgggttctactacggtatggacgtctggggccaagggaccacggtcaccgtctcctca

SEQ ID NO: 58 - protein, 120 aa, Homo sapiens
QVQLQESGPGLVKPSQTLSLTCTVSGGSINSGGYYWSWIRQHPGKGLEWIGYIYYSGSSYYNPSLKSRVTISVDTSKNQFSLKLSSVTAADTAVYYCARDRGHYYGMDVWGQGTTVTVSS

SEQ ID NO: 59 - DNA, 360 bp, Homo sapiens
caggtgcagctgcaggagtcgggcccaggactggtgaagccttcacagaccctgtccctcacctgcactgtctctggtggctccatcaatagtggtggttactactggagctggatccgccagcacccagggaagggcctggagtggattgggtacatctattacagtgggagcagctactacaacccgtccctcaagagtcgagttaccatatcagttgacacgtctaagaaccagttctccctgaagctgagttctgtgactgccgcggacacggccgtgtattactgtgcgagagatcgggggcactactacggtatggacgtctggggccaagggaccacggtcaccgtctcctca

SEQ ID NO: 60 - protein, 123 aa, Homo sapiens
QVQLVESGGGVVQPGRSLRLSCAASGFTFSSYGMHWVRQAPGKGLEWVALIWYDGSNKYYADSVKGRFTISRDNSKNTLYLQMNSLRAEDTAVYYCARENTVTIYYNYGMDVWGQGTTVTVSS

SEQ ID NO: 61 - protein, 116 aa, Artificial
  Consensus Sequence
  MISC_FEATURE (3)..(3): Xaa can be Val or Glu
  MISC_FEATURE (25)..(25): Xaa can be Asn or Ser
  MISC_FEATURE (38)..(38): Xaa can be Gln or Leu
  MISC_FEATURE (55)..(55): Xaa can be Ile or Thr
  MISC_FEATURE (61)..(61): Xaa can be Asp or Glu
  MISC_FEATURE (77)..(77): Xaa can be Tyr or Ser
  MISC_FEATURE (101)..(101): Xaa can be Ser or Asn
QPXLTQPPSASASLGASVTLTCTLXSGYSDYKVDWYQXRPGKGPRFVMRVGTGGXVGSKGXGIPDRFSVLGSGLNRXLTIKNIQEEDESDYHCGADHGSGXNFVYVFGTGTKVTVL

SEQ ID NO: 62 - protein, 14 aa, Homo sapiens
TGSSSNTGAGYDVH

SEQ ID NO: 63 - protein, 7 aa, Homo sapiens
GSGNRPS

SEQ ID NO: 64 - protein, 11 aa, Homo sapiens
QSYDSSLSGWV

SEQ ID NO: 65 - protein, 14 aa, Homo sapiens
TGSSSNIGAGYDVH

SEQ ID NO: 66 - protein, 7 aa, Homo sapiens
GSNNRPS

SEQ ID NO: 67 - protein, 9 aa, Homo sapiens
MIWHSSASV

SEQ ID NO: 68 - protein, 14 aa, Homo sapiens
TLRSGINVGTYRIY

SEQ ID NO: 69 - protein, 11 aa, Homo sapiens
YKSDSDKQQGS

SEQ ID NO: 70 - protein, 13 aa, Homo sapiens
GADHGSGSNFVYV

SEQ ID NO: 71 - protein, 11 aa, Homo sapiens
TLNSGYSDYKV

SEQ ID NO: 72 - protein, 12 aa, Homo sapiens
VGTGGIVGSKGD

SEQ ID NO: 73 - protein, 13 aa, Homo sapiens
GADHGSGNNFVYV

SEQ ID NO: 74 - protein, 11 aa, Homo sapiens
TLSSGYSDYKV

SEQ ID NO: 75 - protein, 12 aa, Homo sapiens
VGTGGIVGSKGE

SEQ ID NO: 76 - protein, 9 aa, Homo sapiens
QQANSFPFT

SEQ ID NO: 77 - protein, 11 aa, Homo sapiens
RASQGFSGWLA

SEQ ID NO: 78 - protein, 12 aa, Homo sapiens
VGTGGTVGSKGE

SEQ ID NO: 79 - protein, 9 aa, Homo sapiens
QQATSFPLT

SEQ ID NO: 80 - protein, 11 aa, Homo sapiens
RASQVISSWLA

SEQ ID NO: 81 - protein, 7 aa, Homo sapiens
AASSLQS

SEQ ID NO: 82 - protein, 9 aa, Homo sapiens
QQADSFPPT

SEQ ID NO: 83 - protein, 11 aa, Homo sapiens
RASQVISSWFA

SEQ ID NO: 84 - protein, 9 aa, Homo sapiens
LQHNSYPPT

SEQ ID NO: 85 - protein, 11 aa, Homo sapiens
RASQGSSSWFA

SEQ ID NO: 86 - protein, 11 aa, Homo sapiens
RASQGISSWFA

SEQ ID NO: 87 - protein, 11 aa, Homo sapiens
RAGQVISSWLA

SEQ ID NO: 88 - protein, 11 aa, Homo sapiens
RASQGIAGWLA

SEQ ID NO: 89 - protein, 11 aa, Homo sapiens
RASQGIRNDLG

SEQ ID NO: 90 - protein, 17 aa, Homo sapiens
LIWYDGSNKYYADSVKG

SEQ ID NO: 91 - protein, 5 aa, Homo sapiens
SYGMH

SEQ ID NO: 92 - protein, 17 aa, Homo sapiens
VIWYDGSNEYYADSVKG

SEQ ID NO: 93 - protein, 15 aa, Homo sapiens
DRGYTSSWYPDAFDI

SEQ ID NO: 94 - protein, 5 aa, Homo sapiens
SYAMH

SEQ ID NO: 95 - protein, 17 aa, Homo sapiens
VIWYDGSNKYYADSVKG

SEQ ID NO: 96 - protein, 15 aa, Homo sapiens
DRGYSSSWYPDAFDI

SEQ ID NO: 97 - protein, 5 aa, Homo sapiens
TYSMN

SEQ ID NO: 98 - protein, 17 aa, Homo sapiens
VISFDGSLKYYADSVKG

SEQ ID NO: 99 - protein, 12 aa, Homo sapiens
ERTTLSGSYFDY

SEQ ID NO: 100 - protein, 5 aa, Homo sapiens
SYSMN

SEQ ID NO: 101 - protein, 17 aa, Homo sapiens
VISHDGSIKYYADSVKG

SEQ ID NO: 102 - protein, 16 aa, Homo sapiens
RIAAAGGFHYYYALDV

SEQ ID NO: 103 - protein, 5 aa, Homo sapiens
SFSMN

SEQ ID NO: 104 - protein, 17 aa, Homo sapiens
YISSRSSTIYIADSVKG

SEQ ID NO: 105 - protein, 16 aa, Homo sapiens
RIAAAGPWGYYYAMDV

SEQ ID NO: 106 - protein, 7 aa, Homo sapiens
SGGYYWT

SEQ ID NO: 107 - protein, 17 aa, Homo sapiens
YISSSSSTRYHADSVKG

SEQ ID NO: 108 - protein, 10 aa, Homo sapiens
NRGYYYGMDV

SEQ ID NO: 109 - protein, 7 aa, Homo sapiens
SGGYYWS

SEQ ID NO: 110 - protein, 17 aa, Homo sapiens
YISSRSSTIYYADSVKG

SEQ ID NO: 111 - protein, 10 aa, Homo sapiens
NRGFYYGMDV

SEQ ID NO: 112 - protein, 5 aa, Homo sapiens
SYFWS

SEQ ID NO: 113 - protein, 16 aa, Homo sapiens
YIYYSGNTYYNPSLKS

SEQ ID NO: 114 - protein, 10 aa, Homo sapiens
DRGHYYGMDV

SEQ ID NO: 115 - protein, 5 aa, Homo sapiens
TYYWS

SEQ ID NO: 116 - protein, 16 aa, Homo sapiens
HIHYSGNTYYNPSLKS

SEQ ID NO: 117 - protein, 10 aa, Homo sapiens
DRGSYYGSDY

SEQ ID NO: 118 - protein, 16 aa, Homo sapiens
YIYYSGSTYYNPSLKS

SEQ ID NO: 119 - protein, 10 aa, Homo sapiens
DRGYYYGVDV

SEQ ID NO: 120 - protein, 16 aa, Homo sapiens
YIYYSGSSYYNPSLKS

SEQ ID NO: 121 - protein, 16 aa, Homo sapiens
YIYYSGSTNYNPSLKS

SEQ ID NO: 122 - protein, 16 aa, Homo sapiens
LIYTSGSTNYNPSLKS

SEQ ID NO: 123 - protein, 11 aa, Artificial
  Consensus sequence
  MISC_FEATURE (5)..(5): Xaa can be Gly or Val
  MISC_FEATURE (6)..(6): Xaa can be Ile, Phe or Ser
  MISC_FEATURE (8)..(8): Xaa can be Ser or Gly
  MISC_FEATURE (10)..(10): Xaa can be  Phe or Leu.
RASQXXSXWXA

SEQ ID NO: 124 - protein, 12 aa, Artificial
  Consensus sequence
  MISC_FEATURE (3)..(3): Xaa can be Asn or Ser
TLXSGYSDYKVD

SEQ ID NO: 125 - protein, 14 aa, Artificial
  Consensus sequence
  MISC_FEATURE (7)..(7): Xaa can be  Ile or Thr
TGSSSNXGAGYDVH

SEQ ID NO: 126 - protein, 12 aa, Artificial
  Consensus sequence
  MISC_FEATURE (6)..(6): Xaa can be Ile or Thr
  MISC_FEATURE (12)..(12): Xaa can be Asp or Glu
VGTGGXVGSKGX

SEQ ID NO: 127 - protein, 7 aa, Artificial
  Consensus sequence
  MISC_FEATURE (3)..(3): Xaa can be Asn or Gly
GSXNRPS

SEQ ID NO: 128 - protein, 13 aa, Artificial
  Consensus sequence
  MISC_FEATURE (8)..(8): Xaa can be Ser or Asn
GADHGSGXNFVYV

SEQ ID NO: 129 - protein, 7 aa, Artificial
  Consensus sequence
  MISC_FEATURE (7)..(7): Xaa can be Ser or Thr
SGGYYWX

SEQ ID NO: 130 - protein, 5 aa, Artificial
  Consensus sequence
  MISC_FEATURE (3)..(3): Xaa can be Gly or Ala
SYXMH

SEQ ID NO: 131 - protein, 5 aa, Artificial
  Consensus sequence
  MISC_FEATURE (1)..(1): Xaa can be Ser or Thr
  MISC_FEATURE (1)..(1): Xaa can be Ser or Thr
  MISC_FEATURE (2)..(2): Xaa can be Tyr or Phe
XXSMN

SEQ ID NO: 132 - protein, 16 aa, Artificial
  Consensus sequence
  MISC_FEATURE (1)..(1): Xaa can be Tyr or His
  MISC_FEATURE (3)..(3): Xaa can be Thr or His
  MISC_FEATURE (7)..(7): Xaa can be Ser or Asn
  MISC_FEATURE (8)..(8): Xaa can be Thr or Ser
XIXYSGXXYYNPSLKS

SEQ ID NO: 133 - protein, 17 aa, Artificial
  Consensus sequence
  MISC_FEATURE (4)..(4): Xaa can be Phe or His
  MISC_FEATURE (8)..(8): Xaa can be Leu or Thr
VISXDGSXKYYADSVKG

SEQ ID NO: 134 - protein, 17 aa, Artificial
  Consensus sequence
  MISC_FEATURE (5)..(5): Xaa can be Arg or Ser
  MISC_FEATURE (9)..(9): Xaa can be Ile or Arg
  MISC_FEATURE (11)..(11): Xaa can be Ile, His or Try
YISSXSSTXYXADSVKG

SEQ ID NO: 135 - protein, 17 aa, Artificial
  Consensus sequence
  MISC_FEATURE (9)..(9): Xaa can be Lys or Glu
VIWYDGSNXYYADSVKG

SEQ ID NO: 136 - protein, 10 aa, Artificial
  Consensus sequence
  MISC_FEATURE (1)..(1): Xaa can be Asn or Asp
  MISC_FEATURE (4)..(4): Xaa can be His, Tyr or Phe
XRGXYYGMDV

SEQ ID NO: 137 - protein, 16 aa, Artificial
  Consensus sequence
  MISC_FEATURE (7)..(7): Xaa can be Gly or Phe
  MISC_FEATURE (8)..(8): Xaa can be Phe or Trp
  MISC_FEATURE (9)..(9): Xaa can be His or Gly
  MISC_FEATURE (14)..(14): Xaa can be Leu and Met
RIAAAGXXXYYYAXDV

SEQ ID NO: 138 - protein, 15 aa, Artificial
  Consensus sequence
  MISC_FEATURE (5)..(5): Xaa can be Ser or Thr
DRGYXSSWYPDAFDI

SEQ ID NO: 139 - protein, 112 aa, Artificial
  Consensus Sequence
  MISC_FEATURE (29)..(29): Xaa can be Ile or Thr
  MISC_FEATURE (41)..(41): Xaa can be Val or Leu
  MISC_FEATURE (54)..(54): Xaa can be Gly or Asn
  MISC_FEATURE (107)..(107): Xaa can be Arg or Lys
QSVLTQPPSVSGAPGQRVTISCTGSSSNXGAGYDVHWYQQXPGTAPKLLIYGSXNRPSGVPDRFSGSKSGTSASLAITGLQAEDEADYYCQSYDSSLSGWVFGGGTXRLTVL

SEQ ID NO: 140 - protein, 120 aa, Artificial
  Consensus Sequence
  MISC_FEATURE (30)..(30): Xaa can be And or Ser
  MISC_FEATURE (37)..(37): Xaa can be Ser or Thr
  MISC_FEATURE (52)..(52): Xaa can be Tyr or His
  MISC_FEATURE (54)..(54): Xaa can be Tyr or His
  MISC_FEATURE (58)..(58): Xaa can be Ser or Asn
  MISC_FEATURE (59)..(59): Xaa can be Ser or Asn
  MISC_FEATURE (69)..(69): Xaa can be Ser or Thr
  MISC_FEATURE (71)..(71): Xaa can be Val or Ile
  MISC_FEATURE (77)..(77): Xaa can be Ile or Met
  MISC_FEATURE (83)..(83): Xaa can be Lys or Gln
  MISC_FEATURE (99)..(99): Xaa can be Arg or Lys
  MISC_FEATURE (100)..(100): Xaa can be Asp or Asn
  MISC_FEATURE (103)..(103): Xaa can be His, Phe or Try
QVQLQESGPGLVKPSQTLSLTCTVSGGSIXSGGYYWXWIRQHPGKGLEWIGXIXYSGXXYYNPSLKSRXTXSVDTSXNQFSLXLSSVTAADTAVYYCAXXRGXYYGMDVWGQGTTVTVSS

SEQ ID NO: 141 - protein, 125 aa, Artificial
  Consensus Sequence
  MISC_FEATURE (23)..(23): Xaa can be Ala or Val
  MISC_FEATURE (24)..(24): Xaa can be Ala or Val
  MISC_FEATURE (31)..(31): Xaa can be Thr or Ser
  MISC_FEATURE (32)..(32): Xaa can be Tyr or Phe
  MISC_FEATURE (54)..(54): Xaa can be Ser or Arg
  MISC_FEATURE (58)..(58): Xaa can be Arg or Ile
  MISC_FEATURE (60)..(60): Xaa can be His, Try or Ile
  MISC_FEATURE (105)..(105): Xaa can be Pro or Gly
  MISC_FEATURE (106)..(106): Xaa can be Trp or Phe
  MISC_FEATURE (107)..(107): Xaa can be Gly or His
  MISC_FEATURE (112)..(112): Xaa can be Met or Leu
EVQLVESGGGLVQPGGSLRLSCXXSGFTFSXXSMNWVRQAPGKGLEWVSYISSXSSTXYXADSVKGRFTISRDNAKNSLYLQMNSLRDEDTAVYYCARRIAAAGXXXYYYAXDVWGQGTTVTVSS

SEQ ID NO: 142 - protein, 121 aa, Artificial
  Consensus Sequence
  MISC_FEATURE (33)..(33): Xaa can be Gly or Ala
  MISC_FEATURE (48)..(48): Xaa can be Val or Leu
  MISC_FEATURE (49)..(49): Xaa can be Ala or Ser
  MISC_FEATURE (53)..(53): Xaa can be Phe or His
  MISC_FEATURE (57)..(57): Xaa can be Leu or Ile
QVQLVESGGGVVQPGRSLRLSCAASGFTFSSYXMHWVRQAPGKGLEWXXVISXDGSXKYYADSVKGRFTISRDNSKNTLYLQMNSLRAEDTAVYYCARERTTLSGSYFDYWGQGTLVTVSS

SEQ ID NO: 143 - protein, 124 aa, Artificial
  Consensus Sequence
  MISC_FEATURE (58)..(58): Xaa can be Glu or Lys
  MISC_FEATURE (103)..(103): Xaa can be Thr or Ser
QVQLVESGGGVVQPGRSLRLSCAASGFTFSSYGMHWVRQAPGKGLEWVAVIWYDGSNXYYADSVKGRFTISRDNSKNTLYLQMNSLRAEDTAVYYCARDRGYXSSWYPDAFDIWGQGTMVTVSS

SEQ ID NO: 144 - DNA, 1026 bp, Homo sapiens
aactcggtgaacaactgagggaaccaaaccagagacgcgctgaacagagagaatcaggctcaaagcaagtggaagtgggcagagattccaccaggactggtgcaaggcgcagagccagccagatttgagaagaaggcaaaaagatgctggggagcagagctgtaatgctgctgttgctgctgccctggacagctcagggcagagctgtgcctgggggcagcagccctgcctggactcagtgccagcagctttcacagaagctctgcacactggcctggagtgcacatccactagtgggacacatggatctaagagaagagggagatgaagagactacaaatgatgttccccatatccagtgtggagatggctgtgacccccaaggactcagggacaacagtcagttctgcttgcaaaggatccaccagggtctgattttttatgagaagctgctaggatcggatattttcacaggggagccttctctgctccctgatagccctgtgggccagcttcatgcctccctactgggcctcagccaactcctgcagcctgagggtcaccactgggagactcagcagattccaagcctcagtcccagccagccatggcagcgtctccttctccgcttcaaaatccttcgcagcctccaggcctttgtggctgtagccgcccgggtctttgcccatggagcagcaaccctgagtccctaaaggcagcagctcaaggatggcactcagatctccatggcccagcaaggccaagataaatctaccaccccaggcacctgtgagccaacaggttaattagtccattaattttagtgggacctgcatatgttgaaaattaccaatactgactgacatgtgatgctgacctatgataaggttgagtatttattagatgggaagggaaatttggggattatttatcctcctggggacagtttggggaggattatttattgtatttatattgaattatgtacttttttcaataaagtcttatttttgtggctaaaaaaaa

SEQ ID NO: 145 - protein, 189 aa, Homo sapiens
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 146 - DNA, 1399 bp, Homo sapiens
ctgtttcagggccattggactctccgtcctgcccagagcaagatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttaggttctgatccaggatgaaaatttggaggaaaagtggaagatattaagcaaaatgtttaaagacacaacggaatagacccaaaaagataatttctatctgatttgctttaaaacgtttttttaggatcacaatgatatctttgctgtatttgtatagttagatgctaaatgctcattgaaacaatcagctaatttatgtatagattttccagctctcaagttgccatgggccttcatgctatttaaatatttaagtaatttatgtatttattagtatattactgttatttaacgtttgtctgccaggatgtatggaatgtttcatactcttatgacctgatccatcaggatcagtccctattatgcaaaat

SEQ ID NO: 147 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 148 - DNA, 2826 bp, Homo sapiens
acaagggtggcagcctggctctgaagtggaattatgtgcttcaaacaggttgaaagagggaaacagtcttttcctgcttccagacatgaatcaggtcactattcaatgggatgcagtaatagccctttacatactcttcagctggtgtcatggaggaattacaaatataaactgctctggccacatctgggtagaaccagccacaatttttaagatgggtatgaatatctctatatattgccaagcagcaattaagaactgccaaccaaggaaacttcatttttataaaaatggcatcaaagaaagatttcaaatcacaaggattaataaaacaacagctcggctttggtataaaaactttctggaaccacatgcttctatgtactgcactgctgaatgtcccaaacattttcaagagacactgatatgtggaaaagacatttcttctggatatccgccagatattcctgatgaagtaacctgtgtcatttatgaatattcaggcaacatgacttgcacctggaatgctgggaagctcacctacatagacacaaaatacgtggtacatgtgaagagtttagagacagaagaagagcaacagtatctcacctcaagctatattaacatctccactgattcattacaaggtggcaagaagtacttggtttgggtccaagcagcaaacgcactaggcatggaagagtcaaaacaactgcaaattcacctggatgatatagtgataccttctgcagccgtcatttccagggctgagactataaatgctacagtgcccaagaccataatttattgggatagtcaaacaacaattgaaaaggtttcctgtgaaatgagatacaaggctacaacaaaccaaacttggaatgttaaagaatttgacaccaattttacatatgtgcaacagtcagaattctacttggagccaaacattaagtacgtatttcaagtgagatgtcaagaaacaggcaaaaggtactggcagccttggagttcactgttttttcataaaacacctgaaacagttccccaggtcacatcaaaagcattccaacatgacacatggaattctgggctaacagttgcttccatctctacagggcaccttacttctgacaacagaggagacattggacttttattgggaatgatcgtctttgctgttatgttgtcaattctttctttgattgggatatttaacagatcattccgaactgggattaaaagaaggatcttattgttaataccaaagtggctttatgaagatattcctaatatgaaaaacagcaatgttgtgaaaatgctacaggaaaatagtgaacttatgaataataattccagtgagcaggtcctatatgttgatcccatgattacagagataaaagaaatcttcatcccagaacacaagcctacagactacaagaaggagaatacaggacccctggagacaagagactacccgcaaaactcgctattcgacaatactacagttgtatatattcctgatctcaacactggatataaaccccaaatttcaaattttctgcctgagggaagccatctcagcaataataatgaaattacttccttaacacttaaaccaccagttgattccttagactcaggaaataatcccaggttacaaaagcatcctaattttgctttttctgtttcaagtgtgaattcactaagcaacacaatatttcttggagaattaagcctcatattaaatcaaggagaatgcagttctcctgacatacaaaactcagtagaggaggaaaccaccatgcttttggaaaatgattcacccagtgaaactattccagaacagaccctgcttcctgatgaatttgtctcctgtttggggatcgtgaatgaggagttgccatctattaatacttattttccacaaaatattttggaaagccacttcaataggatttcactcttggaaaagtagagctgtgtggtcaaaatcaatatgagaaagctgccttgcaatctgaacttgggttttccctgcaatagaaattgaattctgcctctttttgaaaaaaatgtattcacatacaaatcttcacatggacacatgttttcatttcccttggataaatacctaggtaggggattgctgggccatatgataagcatatgtttcagttctaccaatcttgtttccagagtagtgacatttctgtgctcctaccatcaccatgtaagaattcccgggagctccatgcctttttaattttagccattcttctgcctcatttcttaaaattagagaattaaggtcccgaaggtggaacatgcttcatggtcacacatacaggcacaaaaacagcattatgtggacgcctcatgtattttttatagagtcaactatttcctctttattttccctcattgaaagatgcaaaacagctctctattgtgtacagaaagggtaaataatgcaaaatacctggtagtaaaataaatgctgaaaattttcctttaaaatagaatcattaggccaggcgtggtggctcatgcttgtaatcccagcactttggtaggctgaggtaggtggatcacctgaggtcaggagttcgagtccagcctggccaatatgctgaaaccctgtctctactaaaattacaaaaattagccggccatggtggcaggtgcttgtaatcccagctacttgggaggctgaggcaggagaatcacttgaaccaggaaggcagaggttgcactgagctgagattgtgccactgcactccagcctgggcaacaagagcaaaactctgtctggaaaaaaaaaaaaaa

SEQ ID NO: 149 - protein, 629 aa, Homo sapiens
MNQVTIQWDAVIALYILFSWCHGGITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSLFFHKTPETVPQVTSKAFQHDTWNSGLTVASISTGHLTSDNRGDIGLLLGMIVFAVMLSILSLIGIFNRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 150 - DNA, 2100 bp, Homo sapiens
ggtggctgaacctcgcaggtggcagagaggctcccctggggctgtggggctctacgtggatccgatggagccgctggtgacctgggtggtccccctcctcttcctcttcctgctgtccaggcagggcgctgcctgcagaaccagtgagtgctgttttcaggacccgccatatccggatgcagactcaggctcggcctcgggccctagggacctgagatgctatcggatatccagtgatcgttacgagtgctcctggcagtatgagggtcccacagctggggtcagccacttcctgcggtgttgccttagctccgggcgctgctgctacttcgccgccggctcagccaccaggctgcagttctccgaccaggctggggtgtctgtgctgtacactgtcacactctgggtggaatcctgggccaggaaccagacagagaagtctcctgaggtgaccctgcagctctacaactcagttaaatatgagcctcctctgggagacatcaaggtgtccaagttggccgggcagctgcgtatggagtgggagaccccggataaccaggttggtgctgaggtgcagttccggcaccggacacccagcagcccatggaagttgggcgactgcggacctcaggatgatgatactgagtcctgcctctgccccctggagatgaatgtggcccaggaattccagctccgacgacggcagctggggagccaaggaagttcctggagcaagtggagcagccccgtgtgcgttccccctgaaaaccccccacagcctcaggtgagattctcggtggagcagctgggccaggatgggaggaggcggctgaccctgaaagagcagccaacccagctggagcttccagaaggctgtcaagggctggcgcctggcacggaggtcacttaccgactacagctccacatgctgtcctgcccgtgtaaggccaaggccaccaggaccctgcacctggggaagatgccctatctctcgggtgctgcctacaacgtggctgtcatctcctcgaaccaatttggtcctggcctgaaccagacgtggcacattcctgccgacacccacacagaaccagtggctctgaatatcagcgtcggaaccaacgggaccaccatgtattggccagcccgggctcagagcatgacgtattgcattgaatggcagcctgtgggccaggacgggggccttgccacctgcagcctgactgcgccgcaagacccggatccggctggaatggcaacctacagctggagtcgagagtctggggcaatggggcaggaaaagtgttactacattaccatctttgcctctgcgcaccccgagaagctcaccttgtggtctacggtcctgtccacctaccactttgggggcaatgcctcagcagctgggacaccgcaccacgtctcggtgaagaatcatagcttggactctgtgtctgtggactgggcaccatccctgctgagcacctgtcccggcgtcctaaaggagtatgttgtccgctgccgagatgaagacagcaaacaggtgtcagagcatcccgtgcagcccacagagacccaagttaccctcagtggcctgcgggctggtgtagcctacacggtgcaggtgcgagcagacacagcgtggctgaggggtgtctggagccagccccagcgcttcagcatcgaagtgcaggtttctgattggctcatcttcttcgcctccctggggagcttcctgagcatccttctcgtgggcgtccttggctaccttggcctgaacagggccgcacggcacctgtgcccgccgctgcccacaccctgtgccagctccgccattgagttccctggagggaaggagacttggcagtggatcaacccagtggacttccaggaagaggcatccctgcaggaggccctggtggtagagatgtcctgggacaaaggcgagaggactgagcctctcgagaagacagagctacctgagggtgcccctgagctggccctggatacagagttgtccttggaggatggagacaggtgcaaggccaagatgtgatcgttgaggctcagagagggtgagtgactcgcccgaggctacgtagc

SEQ ID NO: 151 - protein, 662 aa, Homo sapiens
MEPLVTWVVPLLFLFLLSRQGAACRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 152 - protein, 6 aa, Artificial Sequence
  His Tag
HHHHHH

SEQ ID NO: 153 - protein, 124 aa, Homo sapiens; identical to SEQ ID NO: 31

SEQ ID NO: 154 - protein, 111 aa, Homo sapiens; identical to SEQ ID NO: 1